SEQUENCE LISTING (184 sequences)

SEQ ID NO: 1 - protein, 11 aa, Homo sapiens
TEYKLVVVGAG

SEQ ID NO: 2 - protein, 14 aa, Homo sapiens
TEYKLVVVGAVGVG

SEQ ID NO: 3 - protein, 13 aa, Homo sapiens
TEYKLVVVGACGV

SEQ ID NO: 4 - protein, 13 aa, Homo sapiens
TEYKLVVVGAAGV

SEQ ID NO: 5 - protein, 12 aa, Homo sapiens
TEYKLVVVGASG

SEQ ID NO: 6 - protein, 14 aa, Homo sapiens
TEYKLVVVGACGVG

SEQ ID NO: 7 - protein, 14 aa, Homo sapiens
TEYKLVVVGAAGVG

SEQ ID NO: 8 - protein, 14 aa, Homo sapiens
TEYKLVVVGASGVG

SEQ ID NO: 9 - protein, 13 aa, Homo sapiens
TEYKLVVVGASGV

SEQ ID NO: 10 - protein, 6 aa, Homo sapiens
VVVGAV

SEQ ID NO: 11 - protein, 7 aa, Homo sapiens
LVVVGAV

SEQ ID NO: 12 - protein, 7 aa, Homo sapiens
VVVGAVG

SEQ ID NO: 13 - protein, 9 aa, Homo sapiens
VVVGAVGVG

SEQ ID NO: 14 - protein, 4 aa, Artificial sequence
  Linker
GSGS

SEQ ID NO: 15 - protein, 18 aa, Artificial sequence
  RAS pept-in
KVVVGAVKGSKVVVGAVK

SEQ ID NO: 16 - protein, 20 aa, Artificial sequence
  RAS pept-in
KLVVVGAVKGSKLVVVGAVK

SEQ ID NO: 17 - protein, 20 aa, Artificial sequence
  RAS pept-in
KVVVGAVGKGSKVVVGAVGK

SEQ ID NO: 18 - protein, 24 aa, Artificial sequence
  RAS pept-in
KVVVGAVGVGKGSKVVVGAVGVGK

SEQ ID NO: 19 - protein, 189 aa, Homo sapiens [sequence omitted]

SEQ ID NO: 20 - protein, 188 aa, Homo sapiens [sequence omitted]

SEQ ID NO: 21 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 22 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 23 - protein, 5 aa, Homo sapiens
GAGGV

SEQ ID NO: 24 - protein, 9 aa, Homo sapiens
VVGAGGVGK

SEQ ID NO: 25 - protein, 13 aa, Homo sapiens
LVVVGAGGVGKSA

SEQ ID NO: 26 - protein, 10 aa, Homo sapiens
LVVVGAVGVG

SEQ ID NO: 27 - protein, 10 aa, Homo sapiens
LVVVGACGVG

SEQ ID NO: 28 - protein, 9 aa, Homo sapiens
LVVVGACGV

SEQ ID NO: 29 - protein, 10 aa, Homo sapiens
LVVVGAAGVG

SEQ ID NO: 30 - protein, 9 aa, Homo sapiens
LVVVGAAGV

SEQ ID NO: 31 - protein, 10 aa, Homo sapiens
LVVVGASGVG

SEQ ID NO: 32 - protein, 9 aa, Homo sapiens
LVVVGASGV

SEQ ID NO: 33 - protein, 8 aa, Homo sapiens
LVVVGASG

SEQ ID NO: 34 - protein, 8 aa, Homo sapiens
VVGAVGVG

SEQ ID NO: 35 - protein, 7 aa, Homo sapiens
VGAVGVG

SEQ ID NO: 36 - protein, 9 aa, Homo sapiens
LVVVGAVGV

SEQ ID NO: 37 - protein, 8 aa, Homo sapiens
VVVGAVGV

SEQ ID NO: 38 - protein, 7 aa, Homo sapiens
VVGAVGV

SEQ ID NO: 39 - protein, 6 aa, Homo sapiens
VGAVGV

SEQ ID NO: 40 - protein, 8 aa, Homo sapiens
LVVVGAVG

SEQ ID NO: 41 - protein, 6 aa, Homo sapiens
VVGAVG

SEQ ID NO: 42 - protein, 9 aa, Homo sapiens
VVVGACGVG

SEQ ID NO: 43 - protein, 8 aa, Homo sapiens
VVGACGVG

SEQ ID NO: 44 - protein, 7 aa, Homo sapiens
VGACGVG

SEQ ID NO: 45 - protein, 8 aa, Homo sapiens
VVVGACGV

SEQ ID NO: 46 - protein, 7 aa, Homo sapiens
VVGACGV

SEQ ID NO: 47 - protein, 6 aa, Homo sapiens
VGACGV

SEQ ID NO: 48 - protein, 8 aa, Homo sapiens
LVVVGACG

SEQ ID NO: 49 - protein, 7 aa, Homo sapiens
VVVGACG

SEQ ID NO: 50 - protein, 4 aa, Artificial sequence
  Gatekeeper region
KKKK

SEQ ID NO: 51 - protein, 7 aa, Homo sapiens
LVVVGAC

SEQ ID NO: 52 - protein, 6 aa, Homo sapiens
VVVGAC

SEQ ID NO: 53 - protein, 9 aa, Homo sapiens
VVVGAAGVG

SEQ ID NO: 54 - protein, 8 aa, Homo sapiens
VVGAAGVG

SEQ ID NO: 55 - protein, 7 aa, Homo sapiens
VGAAGVG

SEQ ID NO: 56 - protein, 8 aa, Homo sapiens
VVVGAAGV

SEQ ID NO: 57 - protein, 7 aa, Homo sapiens
VVGAAGV

SEQ ID NO: 58 - protein, 6 aa, Homo sapiens
VGAAGV

SEQ ID NO: 59 - protein, 8 aa, Homo sapiens
LVVVGAAG

SEQ ID NO: 60 - protein, 7 aa, Homo sapiens
VVVGAAG

SEQ ID NO: 61 - protein, 6 aa, Homo sapiens
VVGAAG

SEQ ID NO: 62 - protein, 7 aa, Homo sapiens
LVVVGAA

SEQ ID NO: 63 - protein, 6 aa, Homo sapiens
VVVGAA

SEQ ID NO: 64 - protein, 9 aa, Homo sapiens
VVVGASGVG

SEQ ID NO: 65 - protein, 8 aa, Homo sapiens
VVGASGVG

SEQ ID NO: 66 - protein, 7 aa, Homo sapiens
VGASGVG

SEQ ID NO: 67 - protein, 8 aa, Homo sapiens
VVVGASGV

SEQ ID NO: 68 - protein, 7 aa, Homo sapiens
VVGASGV

SEQ ID NO: 69 - protein, 6 aa, Homo sapiens
VGASGV

SEQ ID NO: 70 - protein, 7 aa, Homo sapiens
VVVGASG

SEQ ID NO: 71 - protein, 6 aa, Homo sapiens
VVGASG

SEQ ID NO: 72 - protein, 7 aa, Homo sapiens
LVVVGAS

SEQ ID NO: 73 - protein, 6 aa, Homo sapiens
VVVGAS

SEQ ID NO: 74 - protein, 8 aa, Artificial sequence
  FLAG tag
DYKDDDDK

SEQ ID NO: 75 - protein, 7 aa, Homo sapiens
ALTIQLI

SEQ ID NO: 76 - protein, 9 aa, Homo sapiens
GFLCVFAIN

SEQ ID NO: 77 - protein, 5 aa, Homo sapiens
MVLVG

SEQ ID NO: 78 - protein, 6 aa, Homo sapiens
AFYTLV

SEQ ID NO: 79 - protein, 10 aa, Homo sapiens
TEYKLVVVGA

SEQ ID NO: 80 - protein, 18 aa, Artificial sequence
  RAS pept-in
KLSVFAIKGSKLSVFAIK

SEQ ID NO: 81 - protein, 13 aa, Homo sapiens
TEYKLVVVGAGCV

SEQ ID NO: 82 - protein, 14 aa, Homo sapiens
TEYKLVVVGAGVVG

SEQ ID NO: 83 - protein, 12 aa, Homo sapiens
TEYKLVVVGAGS

SEQ ID NO: 84 - protein, 14 aa, Homo sapiens
TEYKLVVVGAGCVG

SEQ ID NO: 85 - protein, 14 aa, Homo sapiens
TEYKLVVVGAGSVG

SEQ ID NO: 86 - protein, 13 aa, Homo sapiens
TEYKLVVVGAGSV

SEQ ID NO: 87 - protein, 5 aa, Homo sapiens
AGGVG

SEQ ID NO: 88 - protein, 9 aa, Homo sapiens
VGAGGVGKS

SEQ ID NO: 89 - protein, 13 aa, Homo sapiens
VVVGAGGVGKSAL

SEQ ID NO: 90 - protein, 10 aa, Homo sapiens
LVVVGAGVVG

SEQ ID NO: 91 - protein, 14 aa, Homo sapiens
TEYKLVVVGAGCVG

SEQ ID NO: 92 - protein, 10 aa, Homo sapiens
LVVVGAGCVG

SEQ ID NO: 93 - protein, 9 aa, Homo sapiens
LVVVGAGCV

SEQ ID NO: 94 - protein, 14 aa, Homo sapiens
TEYKLVVVGAGSVG

SEQ ID NO: 95 - protein, 13 aa, Homo sapiens
TEYKLVVVGAGSV

SEQ ID NO: 96 - protein, 10 aa, Homo sapiens
LVVVGAGSVG

SEQ ID NO: 97 - protein, 9 aa, Homo sapiens
LVVVGAGSV

SEQ ID NO: 98 - protein, 8 aa, Homo sapiens
LVVVGAGS

SEQ ID NO: 99 - protein, 7 aa, Homo sapiens
VVVGAGV

SEQ ID NO: 100 - protein, 8 aa, Homo sapiens
LVVVGAGV

SEQ ID NO: 101 - protein, 8 aa, Homo sapiens
VVVGAGVV

SEQ ID NO: 102 - protein, 9 aa, Homo sapiens
VVVGAGVVG

SEQ ID NO: 103 - protein, 8 aa, Homo sapiens
VVGAGVVG

SEQ ID NO: 104 - protein, 7 aa, Homo sapiens
VGAGVVG

SEQ ID NO: 105 - protein, 6 aa, Homo sapiens
GAGVVG

SEQ ID NO: 106 - protein, 9 aa, Homo sapiens
LVVVGAGVV

SEQ ID NO: 107 - protein, 8 aa, Homo sapiens
VVVGAGVV

SEQ ID NO: 108 - protein, 7 aa, Homo sapiens
VVGAGVV

SEQ ID NO: 109 - protein, 6 aa, Homo sapiens
VGAGVV

SEQ ID NO: 110 - protein, 6 aa, Homo sapiens
VVGAGV

SEQ ID NO: 111 - protein, 7 aa, Homo sapiens
VGAGCVG

SEQ ID NO: 112 - protein, 6 aa, Homo sapiens
GAGCVG

SEQ ID NO: 113 - protein, 8 aa, Homo sapiens
VVVGAGCV

SEQ ID NO: 114 - protein, 7 aa, Homo sapiens
VVGAGCV

SEQ ID NO: 115 - protein, 6 aa, Homo sapiens
VGAGCV

SEQ ID NO: 116 - protein, 8 aa, Homo sapiens
LVVVGAGC

SEQ ID NO: 117 - protein, 7 aa, Homo sapiens
VVVGAGC

SEQ ID NO: 118 - protein, 6 aa, Homo sapiens
VVGAGC

SEQ ID NO: 119 - protein, 9 aa, Homo sapiens
VVVGAGSVG

SEQ ID NO: 120 - protein, 8 aa, Homo sapiens
VVGAGSVG

SEQ ID NO: 121 - protein, 7 aa, Homo sapiens
VGAGSVG

SEQ ID NO: 122 - protein, 6 aa, Homo sapiens
GAGSVG

SEQ ID NO: 123 - protein, 8 aa, Homo sapiens
VVVGAGSV

SEQ ID NO: 124 - protein, 7 aa, Homo sapiens
VVGAGSV

SEQ ID NO: 125 - protein, 6 aa, Homo sapiens
VGAGSV

SEQ ID NO: 126 - protein, 7 aa, Homo sapiens
VVVGAGS

SEQ ID NO: 127 - protein, 6 aa, Homo sapiens
VVGAGS

SEQ ID NO: 128 - protein, 20 aa, Artificial sequence
  RAS pept-in
KVVVGAGVKGSKVVVGAGVK

SEQ ID NO: 129 - protein, 22 aa, Artificial sequence
  RAS pept-in
KLVVVGAGVKGSKLVVVGAGVK

SEQ ID NO: 130 - protein, 22 aa, Artificial sequence
  RAS pept-in
KVVVGAGVVKGSKVVVGAGVVK

SEQ ID NO: 131 - protein, 24 aa, Artificial sequence
  RAS pept-in
KVVVGAGVVGKGSKVVVGAGVVGK

SEQ ID NO: 132 - protein, 10 aa, Homo sapiens
TEYKLVVVGA

SEQ ID NO: 133 - protein, 4 aa, Artificial sequence
  Gatekeeper region
RRRR

SEQ ID NO: 134 - protein, 4 aa, Artificial sequence
  Gatekeeper region
DDDD

SEQ ID NO: 135 - protein, 4 aa, Artificial sequence
  Gatekeeper region
EEEE

SEQ ID NO: 136 - protein, 4 aa, Artificial sequence
  Gatekeeper region
KRKR

SEQ ID NO: 137 - protein, 4 aa, Artificial sequence
  Gatekeeper region
KRRK

SEQ ID NO: 138 - protein, 4 aa, Artificial sequence
  Gatekeeper region
RKKR

SEQ ID NO: 139 - protein, 4 aa, Artificial sequence
  Gatekeeper region
DEDE

SEQ ID NO: 140 - protein, 4 aa, Artificial sequence
  Gatekeeper region
DEED

SEQ ID NO: 141 - protein, 4 aa, Artificial sequence
  Gatekeeper region
EDDE

SEQ ID NO: 142 - protein, 4 aa, Artificial sequence
  Linker
GGGG

SEQ ID NO: 143 - protein, 4 aa, Artificial sequence
  Linker
SSSS

SEQ ID NO: 144 - protein, 4 aa, Artificial sequence
  Linker
GGGS

SEQ ID NO: 145 - protein, 4 aa, Artificial sequence
  Linker
GGSG

SEQ ID NO: 146 - protein, 4 aa, Artificial sequence
  Linker
GSGG

SEQ ID NO: 147 - protein, 4 aa, Artificial sequence
  Linker
SGGG

SEQ ID NO: 148 - protein, 4 aa, Artificial sequence
  Linker
GGSS

SEQ ID NO: 149 - protein, 4 aa, Artificial sequence
  Linker
GSSG

SEQ ID NO: 150 - protein, 4 aa, Artificial sequence
  Linker
SSGG

SEQ ID NO: 151 - protein, 4 aa, Artificial sequence
  Linker
SGSG

SEQ ID NO: 152 - protein, 5 aa, Artificial sequence
  Linker
GSGSG

SEQ ID NO: 153 - protein, 5 aa, Artificial sequence
  Linker
SGSGS

SEQ ID NO: 154 - protein, 4 aa, Artificial sequence
  Linker
PPPP

SEQ ID NO: 155 - protein, 6 aa, Homo sapiens
LSVFAI

SEQ ID NO: 156 - protein, 18 aa, Artificial sequence
  RAS pept-in
  MISC_FEATURE (1)..(1): D-lysine
  MISC_FEATURE (18)..(18): D-lysine
XLSVFAIKGSKLSVFAIX

SEQ ID NO: 157 - protein, 18 aa, Artificial sequence
  RAS pept-in
  MISC_FEATURE (1)..(1): D-lysine
  MISC_FEATURE (18)..(18): D-lysine
XVVVGAVKGSKVVVGAVX

SEQ ID NO: 158 - protein, 20 aa, Artificial sequence
  RAS pept-in
  MISC_FEATURE (1)..(1): D-lysine
  MISC_FEATURE (20)..(20): D-lysine
XVVVGAVGKGSKVVVGAVGX

SEQ ID NO: 159 - protein, 24 aa, Artificial sequence
  RAS pept-in
  MISC_FEATURE (1)..(1): D-lysine
  MISC_FEATURE (24)..(24): D-lysine
XVVVGAVGVGKGSKVVVGAVGVGX

SEQ ID NO: 160 - protein, 18 aa, Artificial sequence
  RAS pept-in
  MOD_RES (1)..(1): Dpm
  MOD_RES (18)..(18): Dpm
XLSVFAIKGSKLSVFAIX

SEQ ID NO: 161 - protein, 18 aa, Artificial sequence
  RAS pept-in
  MOD_RES (1)..(1): Dpm
  MOD_RES (18)..(18): Dpm
XVVVGAVKGSKVVVGAVX

SEQ ID NO: 162 - protein, 20 aa, Artificial sequence
  RAS pept-in
  MOD_RES (1)..(1): Dpm
  MOD_RES (20)..(20): Dpm
XVVVGAVGKGSKVVVGAVGX

SEQ ID NO: 163 - protein, 24 aa, Artificial sequence
  RAS pept-in
  MOD_RES (1)..(1): Dpm
  MOD_RES (24)..(24): Dpm
XVVVGAVGVGKGSKVVVGAVGVGX

SEQ ID NO: 164 - protein, 18 aa, Artificial sequence
  RAS pept-in
  MISC_FEATURE (1)..(1): citrulline
XVVVGAVKGSKVVVGAVK

SEQ ID NO: 165 - protein, 18 aa, Artificial sequence
  RAS pept-in
  MISC_FEATURE (8)..(8): citrulline
KVVVGAVXGSKVVVGAVK

SEQ ID NO: 166 - protein, 18 aa, Artificial sequence
  RAS pept-in
AVVVGAVKGSKVVVGAVK

SEQ ID NO: 167 - protein, 18 aa, Artificial sequence
  RAS pept-in
KVVVGAVAGSKVVVGAVK

SEQ ID NO: 168 - protein, 18 aa, Artificial sequence
  RAS pept-in
KVVVGAVKGSAVVVGAVK

SEQ ID NO: 169 - protein, 18 aa, Artificial sequence
  RAS pept-in
KVVVGAVKGSKVVVGAVA

SEQ ID NO: 170 - protein, 18 aa, Artificial sequence
  RAS pept-in
AVVVGAVKGSAVVVGAVK

SEQ ID NO: 171 - protein, 18 aa, Artificial sequence
  RAS pept-in
KVVVGAVAGSKVVVGAVA

SEQ ID NO: 172 - protein, 18 aa, Artificial sequence
  RAS pept-in
AVVVGAVAGSKVVVGAVK

SEQ ID NO: 173 - protein, 18 aa, Artificial sequence
  RAS pept-in
KVVVGAVKASKVVVGAVK

SEQ ID NO: 174 - protein, 18 aa, Artificial sequence
  RAS pept-in
KVVVGAVKGAKVVVGAVK

SEQ ID NO: 175 - protein, 20 aa, Artificial sequence
  RAS pept-in
KVVVGAVGKGFKVVVGAVGK

SEQ ID NO: 176 - protein, 20 aa, Artificial sequence
  RAS pept-in
KVVVGAVGKFFKVVVGAVGK

SEQ ID NO: 177 - protein, 16 aa, Artificial sequence
  RAS pept-in
KLSVFAIKKLSVFAIK

SEQ ID NO: 178 - protein, 22 aa, Artificial sequence
  RAS pept-in
KVVVGAVGVGKKVVVGAVGVGK

SEQ ID NO: 179 - protein, 18 aa, Artificial sequence
  RAS pept-in
  MISC_FEATURE (1)..(1): D-lysine
  MISC_FEATURE (2)..(2): D-leucine
  MISC_FEATURE (3)..(3): D-serine
  MISC_FEATURE (4)..(4): D-valine
  MISC_FEATURE (5)..(5): D-phenylalanine
  MISC_FEATURE (6)..(6): D-alanine
  MISC_FEATURE (7)..(7): D-isoleucine
  MISC_FEATURE (8)..(8): D-lysine
  MISC_FEATURE (10)..(10): D-serine
  MISC_FEATURE (11)..(11): D-lysine
  MISC_FEATURE (12)..(12): D-leucine
  MISC_FEATURE (13)..(13): D-serine
  MISC_FEATURE (14)..(14): D-valine
  MISC_FEATURE (15)..(15): D-phenylalanine
  MISC_FEATURE (16)..(16): D-alanine
  MISC_FEATURE (17)..(17): D-isoleucine
  MISC_FEATURE (18)..(18): D-lysine
XXXXXXXXGXXXXXXXXX

SEQ ID NO: 180 - protein, 18 aa, Artificial sequence
  RAS pept-in
  MISC_FEATURE (1)..(1): D-lysine
  MISC_FEATURE (2)..(2): D-valine
  MISC_FEATURE (3)..(3): D-valine
  MISC_FEATURE (4)..(4): D-valine
  MISC_FEATURE (6)..(6): D-alanine
  MISC_FEATURE (7)..(7): D-valine
  MISC_FEATURE (8)..(8): D-lysine
  MISC_FEATURE (10)..(10): D-serine
  MISC_FEATURE (11)..(11): D-lysine
  MISC_FEATURE (12)..(12): D-valine
  MISC_FEATURE (13)..(13): D-valine
  MISC_FEATURE (14)..(14): D-valine
  MISC_FEATURE (16)..(16): D-alanine
  MISC_FEATURE (17)..(17): D-valine
  MISC_FEATURE (18)..(18): D-lysine
XXXXGXXXGXXXXXGXXX

SEQ ID NO: 181 - protein, 24 aa, Artificial sequence
  RAS pept-in
  MISC_FEATURE (1)..(1): D-lysine
  MISC_FEATURE (2)..(2): D-valine
  MISC_FEATURE (3)..(3): D-valine
  MISC_FEATURE (4)..(4): D-valine
  MISC_FEATURE (6)..(6): D-alanine
  MISC_FEATURE (7)..(7): D-valine
  MISC_FEATURE (9)..(9): D-valine
  MISC_FEATURE (11)..(11): D-lysine
  MISC_FEATURE (13)..(13): D-serine
  MISC_FEATURE (14)..(14): D-lysine
  MISC_FEATURE (15)..(15): D-valine
  MISC_FEATURE (16)..(16): D-valine
  MISC_FEATURE (17)..(17): D-valine
  MISC_FEATURE (19)..(19): D-alanine
  MISC_FEATURE (20)..(20): D-valine
  MISC_FEATURE (22)..(22): D-valine
  MISC_FEATURE (24)..(24): D-lysine
XXXXGXXGXGXGXXXXXGXXGXGX

SEQ ID NO: 182 - protein, 10 aa, Artificial sequence
  peptide
KLVVVGAVGV

SEQ ID NO: 183 - protein, 17 aa, Artificial sequence
  peptide
KLVVVGAVGVGKSALTI

SEQ ID NO: 184 - protein, 13 aa, Artificial sequence
  peptide
KLVVVGAVGVGKS

(19) United States
(12) Patent Application Publication
(10) Pub. No.: US 2023/0100941 A1
(43) Pub. Date: Mar. 30, 2023

(54) MOLECULES TARGETING MUTANT RAS PROTEIN

(71) Applicants: AELIN THERAPEUTICS, Leuven (BE); VIB VZW, Gent (BE); KATHOLIEKE UNIVERSITEIT LEUVEN, Leuven (BE)

(72) Inventors: Filip, Maria, Hendrik CLAES, Bierbeek (BE); Joost SCHYMKOWITZ, Meensel-Kiezegem (BE); Frederic ROUSSEAU, Sint-Martens-Bodegem (BE)

(21) Appl. No.: 17/800,738

(22) PCT Filed: Feb. 19, 2021
(86) PCT No.: PCT/EP2021/054118

§ 371 Date: Aug. 18, 2022

(30) Foreign Application Priority Data
Feb. 19, 2020 (EP) 20158306.9

(51) Int. Cl. C07K 7/08; A61P 35/00

(52) U.S. Cl. CPC C07K 7/08; A61P 35/00; A61K 38/00

(57) ABSTRACT

Aspects of the invention concern non-naturally occurring molecules configured to form an intermolecular beta-sheet with a human RAS protein mutated at position 12 or 13 and substantially not with wild-type human RAS protein, as well therapeutic applications thereof.

### FIELD

The invention is broadly in the medical field and more specifically concerns molecules directed to mutant human RAS proteins. The disclosed molecules are particularly useful in therapy, such as in methods of treating neoplastic diseases. The application also teaches methods for making and using the disclosed molecules and compositions comprising the molecules.

### BACKGROUND

RAS proteins belong to small GTPase class of proteins and are involved in cytoplasmic signal transduction pathways regulating diverse normal cellular processes, such as cell growth and division, differentiation and survival. RAS GTPases cycle between the GDP-bound inactive and GTP-bound active states with the help of guanine nucleotide exchange factors (GEFs) that promote activation and GTPase-activating proteins (GAPs) that inactivate RAS by catalysing GTP hydrolysis. Once activated, RAS-GTP binds to and activates a spectrum of downstream effectors with distinct catalytic functions. The three human RAS genes (Kirsten rat sarcoma viral oncogene homolog (KRAS), neuroblastoma RAS viral oncogene homolog (NRAS) and Harvey rat sarcoma viral oncogene homolog (HRAS)) encode four RAS proteins, with two KRAS isoforms that arise from alternative RNA splicing of the KRAS transcript (KRAS4A and KRAS4B).

Certain mutations in RAS genes can lead to the production of permanently activated RAS proteins, leading to active intracellular signalling even in the absence of incoming signals, which can ultimately result in or contribute to neoplastic transformation of cells expressing such mutated RAS proteins. Gain-of-function missense mutations in RAS genes (more than 130 different missense mutations have been reported in RAS genes) are found in about 27% of all human cancers and up to 90% in certain types of cancer, validating mutant RAS genes as very common if not the most common oncogenes driving tumour initiation and maintenance. In human cancers, KRAS is the predominantly mutated RAS isoform (85%), whereas HRAS (4%) and NRAS (11%) are less frequently mutated. Moreover, 98% of the mutations are found at one of three missense-mutation hotspots: G12 (with G12C, G12D, G12S, and G12V mutations being among the most frequent at G12), G13 (with G13C, G13D, G13R, G13S, and G13V mutations being among the most frequent at G13) and Q61 (with Q61H, Q61K, Q61L, and Q61R mutations being among the most frequent at Q61). Conventionally, mutant RAS is considered to be defective in GAP-mediated GTP hydrolysis, which results in an accumulation of constitutively active GTP-bound RAS in cells. See Hobbs et al. J Cell Sci. 2016, vol. 129, 1287-92.

WO 2007/071789A1 and WO2012/123419A1 describe technology allowing for targeted downregulation of proteins of interest, utilising de novo designed peptide-based molecules (referred to therein as “interferors”) comprising at least one β-aggregating sequence which is directed to and can interact with a corresponding β-aggregation prone region (APR) in a protein of interest. Such APRs can be determined in protein sequences using publically available algorithms and computer programs, such as TANGO (Fernandez-Escamilla et al. Nat Biotechnol. 2004, vol. 22, 1302-6, http://tango.embl.de/) or Zyggregator (Pawar et al. J Mol Biol. 2005, vol. 350, 379-92; Tartaglia and Vendruscolo, Chem Soc Rev. 2008, vol. 37, 1395-401).

It was proposed in WO 2007/071789A1 and WO2012/123419A1 that upon contact between a protein of interest comprising an APR in its amino acid sequence and an interferor molecule comprising a β-aggregating sequence corresponding to said APR, a specific β-sheet interaction and co-aggregation occurs between the interferor and the protein of interest, leading to reduced solubility of the protein of interest and its sequestration into aggregates or inclusion bodies, and consequently an effective down-regulation or knock-down of the biological function of said protein of interest.

### SUMMARY

The present invention is at least in part based on the unexpected finding that certain missense mutations at G12 or G13 of human RAS proteins alter the profile of β-aggregation prone regions (APRs) in the human RAS proteins, which can be successfully exploited to design novel molecules targeting specifically such mutant RAS proteins.

In particular, human RAS proteins are predicted to contain 5 APR regions of at least 5 amino acids (see Table 6). The most N-terminal APR (TEYKLVVVGAG, SEQ ID NO: 1) is C-terminally delineated by G12 (underlined) in the wild-type proteins. However, certain G12 missense mutations, such as particularly G12V, G12C, G12A, or G12S enlarge this APR such that the APRs in the respective RAS mutants include not only the mutated residue at position 12 but additionally one or more subsequent residues. Further, certain G13 missense mutations, such as particularly G13V, G13C, or G13S, enlarge this APR such that the APRs in the respective RAS mutants include not only the glycine at position 12 but additionally the mutated residue at position 13 and optionally one or more subsequent residues.

Hence, this APR is predicted to span positions 2-15 and display the sequence TEYKLVVVGAVGVG (SEQ ID NO: 2) in the G12V RAS mutant; to span positions 2-14 and display the sequence TEYKLVVVGACGV (SEQ ID NO: 3) in the G12C RAS mutant; to span positions 2-14 and display the sequence TEYKLVVVGAAGV (SEQ ID NO: 4) in the G12A RAS mutant; and to span positions 2-13 and display the sequence TEYKLVVVGASG (SEQ ID NO: 5) in the G12S RAS mutant. Additionally, at least some mutations at G12 of human RAS, such as in particular the G12V mutation, also significantly increase the predicted aggregation propensity of the corresponding APR. For example, the TANGO algorithm predicts an aggregation score of about 20% for the wild-type APR as set forth in SEQ ID NO: 1, but almost 41% for the APR in the G12V RAS mutant as set forth in SEQ ID NO: 2. Further, this APR is predicted to span positions 2-14 and display the sequence TEYKLVVVGAGCV (SEQ ID NO: 81) in the G13C RAS mutant; to span positions 2-15 and display the sequence TEYKLVVVGAGVVG (SEQ ID NO: 82) in the G13V RAS mutant; and to span positions 2-13 and display the sequence TEYKLVVVGAGS (SEQ ID NO: 83) in the G13S RAS mutant. Additionally, at least some mutations at G13 of human RAS, such as in particular the G13V mutation, also significantly increase the predicted aggregation propensity of the corresponding APR. For example, the TANGO algorithm predicts an aggregation score of about 20% for the wild-type APR as set forth in SEQ ID NO: 1, but almost 41% for the APR in the G13V RAS mutant as set forth in SEQ ID NO: 82.

Having performed extensive experiments, the present inventors now demonstrate that these differences between the N-terminal most APRs predicted in the G12 or G13 mutant RAS proteins and wild-type RAS allow to design molecules comprising β-aggregating sequence(s) which specifically target the APRs found in the G12 or G13 mutant RAS proteins, and which can downregulate the G12 or G13 mutant RAS proteins but substantially not wild-type RAS.

Accordingly, an aspect provides a non-naturally occurring molecule configured to form an intermolecular beta-sheet with a G12 or G13 mutant human RAS protein (or put otherwise, a human RAS protein mutated at G12 or G13, or yet differently, a human RAS protein in which the glycine at position 12 or the glycine at position 13 is mutated) and substantially not with wild-type human RAS protein. The capacity of such molecule to specifically target the G12 or G13 mutant RAS protein for the intermolecular β-sheet formation may manifest in particular as the molecule's ability to downregulate, decrease solubility of and/or induce aggregation or inclusion body formation of the G12 or G13 mutant RAS protein but substantially not of wild-type RAS, such as for example in an appropriate in vitro, cell culture or in vivo setup.

Further aspects provide any molecule as taught herein for use in medicine.

Further aspects provide any molecule as taught herein for use in a method of treating a disease caused by or associated with a G12 or G13 mutation in human RAS protein.

Related aspects provide a method for treating a subject in need thereof, the method comprising administering to the subject a therapeutically effective amount of any molecule as taught herein.

Further aspects provide a pharmaceutical composition comprising any molecule as taught herein. It shall be appreciated that to the extent that the present molecules allow to target non-human mutant RAS molecules, (such as RAS molecules from other eukaryotes, particularly from yeast, fungi or animals, more particularly from animals, even more particularly warm-blooded animals, and still more particularly mammals, such as domesticated animals, farm animals, sport animals, or pets), for example owing to an acceptable degree of sequence identity between the targeted APR in human RAS and a corresponding APR in a non-human RAS, the molecules may also be used in non-human animals similarly as described herein for humans. Hence, medical interventions and pharmaceutical compositions as contemplated herein may also subsume veterinary treatments and compositions for veterinary use. By the same token, the present molecules may lend themselves for a variety of in vitro, in cellulo or in vivo applications (e.g., diagnostics, imaging, use in cell or non-human animal models, research tool use, etc.) not only in human cells or tissues, but also in non-human cells or tissues and in non-human animals. Hence, also provided is an in vitro method for downregulating the amount or biological activity of a mutant RAS in a cell (e.g., a human or non-human cell) expressing, e.g., endogenously or exogenously expressing, the mutant RAS, the method comprising contacting the cell with a RAS targeting pept-in as taught herein or with a nucleic acid molecule encoding the same (an alternative available for polypeptide peptins). Also provided is thus a method for downregulating the amount or biological activity of a mutant RAS in a non-human organism expressing, e.g., endogenously or exogenously expressing, the mutant RAS, the method comprising administering to the organism a RAS targeting pept-in as taught herein or a nucleic acid molecule encoding the same.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 illustrates a screen of RAS-targeting molecules (‘pept-ins’) according to certain embodiments of the present invention on NCI-H441 tumor cell line cultures. (A) Single-dose (25 μM) screen of RAS-targeting pept-ins on adherently growing (2D) NCI-H441 cells. Viability was assessed after 4 days of exposure to the test compounds and normalized to the vehicle condition (30 mM Urea). (B) Single-dose (25 μM) screen of RAS-targeting pept-ins on NCI-H441 cells growing as suspension spheroid cultures (3D). Viability was assessed after 5 days of exposure to the test compounds and normalized to the vehicle condition (30 mM Urea). NT: Not tested. Error bars represent the SD.

FIG. 2 illustrates dose-response and IC₅₀ determination of RAS-targeting molecules (‘pept-ins’) according to certain embodiments of the present invention and a negative control. Pept-ins were tested in a five-point dose-response using a one-in-two dilution series starting from 50 μM as highest dose on adherently growing (2D) NCI-H441 cells. Viability was assessed after three days of exposure to the test compounds and normalized to vehicle conditions. Error bars represent the SD.

FIG. 3 illustrates IC₅₀s of RAS-targeting molecules (‘pept-ins’) according to certain embodiments of the present invention on suspension spheroid cultures. Waterfall plots showing the median IC₅₀s of RAS-targeting pept-ins on suspension spheroid cultures. Pept-ins were tested in a five-point dose-response using a one-in-two dilution series starting from 50 μM as highest dose on spheroid suspension cultures on a set of cell lines with different KRAS mutations. Viability was assessed five days after of exposure to the test compounds. Error bars represent the SD on the median, if applicable.

FIG. 4 illustrates kinetic tinctorial aggregation assays on RAS-targeting molecules (‘pept-ins’) according to certain embodiments of the present invention. Aggregation behaviour of the RAS-targeting pept-ins was studied by performing kinetic tinctorial assays using the amyloid aggregate sensor dyes Thioflavin T (ThT; lower panel) and pentameric formyl thiophene acetic acid (p-FTAA; upper panel). All four biologically active pept-ins showed clear amyloid-aggregation kinetics with both dyes, while the inactive control showed no significant ThT signal and only a slight increase in p-FTAA signal over time.

FIG. 5 illustrates seeding of KRAS G12V by RAS-targeting molecules (‘pept-ins’) according to certain embodiments of the present invention. Seeding experiments of recombinant native KRAS G12V protein was performed with end-stage aggregates (left panels) or sonicated seeds (right panels) of the different KRAS-targeting pept-ins. To this end, pept-ins were allowed to aggregate for 22 hrs. End-stage samples were mixed with recombinant KRAS G12V and aggregation was monitored kinetically using ThT. This approach revealed only minor seeding capacity of these end-stage pept-in aggregates on KRAS G12V. However, upon disruption of the mature aggregates through sonication, potent seeds are formed which efficiently induce aggregation of KRAS G12V.

FIG. 6 illustrates in vitro translation assay showing target selectivity of RAS-targeting molecules (‘pept-ins’) according to certain embodiments of the present invention. In vitro translation assay producing either wild-type or different mutant KRAS in the presence of biotinylated RAS-targeting pept-ins. Streptavidin pull-down was used to capture the biotinylated pept-ins from the translation reaction and pulled-down fraction was probed for KRAS using Western blot. The biotinylated version of pept-in 04-004-N001, i.e. 04-004-N011, which harbours an APR window sequence derived from a wild-type APR, is predicted to target all RAS proteins independently from their mutation status. While efficient pull-down with 04-004-N001 was indeed observed for KRAS wild-type, G12V and G12C, binding to the G12D and G13D mutants appeared to be less efficient. Using the biotinylated versions of the biologically active pept-ins harbouring an APR window containing the G12V mutant site (04-006-N007, 04-015-N026 and 04-033-N003), however, pull-down was only observed for the G12V mutant KRAS and, in the case of 04-015-N026, for G12C mutant KRAS.

FIG. 7 illustrates cellular co-immunoprecipitation assays showing target engagement by RAS-targeting molecules (‘pept-ins’) according to certain embodiments of the present invention. Cellular target engagement of biotinylated pept-ins was assessed using co-immunoprecipitation assay. NCI-H441 cells were treated with 25 μM biotinylated pept-ins overnight after which pept-ins were immunoprecipitated from the lysates using streptavidin-coated beads. Precipitated fractions were probed for KRAS using Western blot. While this approach yielded no detectable KRAS protein in the precipitated fractions from vehicle or negative control peptide-treated conditions, KRAS protein was readily detected in the precipitated fractions from NCI-H441 cells treated with biologically active pept-ins.

FIG. 8 illustrates cellular co-localization between mCherry-labeled KRAS and FITC-labeled RAS-targeting molecules (‘pept-ins’) according to certain embodiments of the present invention. HeLa cells overexpressing mCherry-tagged KRAS G12V were treated with the RAS-targeting FITC-labeled version of pept-in 04-015-N001 (04-015-N032) and imaged 75 min after initial exposure to the pept-in. mCherry-labeled KRAS associates with the pept-in as revealed by the occurrence of inclusion-like perinuclear structures that are positive for both FITC as well as mCherry (white arrows).

FIG. 9 illustrates that RAS-targeting molecules (‘pept-ins’) according to certain embodiments of the present invention lower solubility and total levels of the KRAS protein. NCI-H441 cells were treated with a near IC50 dose (12.5 μM) and a near 2XIC50 dose (25 μM) for 24 hrs. Insoluble proteins in lysates were collected by centrifugation and both soluble and insoluble protein fractions were probed for KRAS on Western blot. This analysis showed that all biologically active RAS-targeting peptides dose-dependently increased the percentage of KRAS in the insoluble fraction while the percentage of insoluble KRAS was comparable between vehicle and negative control peptide treated samples (A). Quantification of total KRAS levels in these samples (i.e. sum of KRAS levels in the soluble and insoluble fraction for each treatment) showed that total KRAS levels were also dose-dependently reduced in the samples treated with the biologically active RAS-targeting pept-ins (B).

FIG. 10 illustrates mutant-selective cellular efficacy using the RASless MEF panel. Graph showing mean ±SD as well as individual assay IC50s from at least three independent experiments assessing the efficacy of the indicated RAS-target pept-ins on a panel of RASless MEFs, expressing either wild-type (WT), mutant G12V or G12C KRAS, or a V600E mutant BRAF in absence of endogenous K-, H-, and NRAS.

FIG. 11 illustrates cellular co-immunoprecipitation assays showing target engagement by RAS-targeting molecules (‘pept-ins’) according to certain embodiments of the present invention. Cellular target engagement of biotinylated pept-ins was assessed using co-immunoprecipitation assay. KRAS wild-type or mutant G12V expressing RASless MEFs. In the RASless MEF-based assay, blots show that the 04-004-derived biotinylated pept-in precipitated both wild-type and mutant G12V KRAS well. The biotinylated versions of the G12V-selective pept-ins, however, show preferential binding to the G12V mutant KRAS protein.

FIG. 12 illustrates flow cytometry assay probing cell death and protein aggregation upon treatment with RAS-targeting pept-ins. NCI-H441 lung adenocarcinoma cells were treated with the indicated RAS-targeting pept-ins and control conditions for 6, 16 or 24hrs. After treatment, cells were collected and stained for cell death (Sytox™ Blue) and protein aggregation (Amytracker™ Red), and next analyzed on a flow cytometer. Scatter plots show Sytox Blue intensity on the Y-axis and Amytracker Red intensity on the X-axis. Hpt: hours post treatment. Treatment with all of the RAS-targeting pept-ins, but not with the control conditions, induced protein aggregation as evidenced by the increase in Amytracker Red signal. Furthermore, this increase in aggregation appears to result in cell death, as indicated by the slower but parallel increase in Sytox Blue.

FIG. 13 illustrates that RAS-targeting pept-ins reduce tumor growth in a xenograft model of KRAS G12V mutant cancer. A xenograft model of human KRAS G12V mutant colorectal cancer, SW620, was used to assess whether in vivo administration of the RAS-targeting pept-ins resulted in reduction of tumor growth. Pept-ins were dosed 3 times per week by intratumoral injection at either 20 or 200 μg once the tumors reached 100-150 mm³. Model response was monitored by a positive control group receiving Irinotecan at 100 mg/kg, once per week for 3 weeks. Group sizes were N=6 for the non-treated group, N=5 for the vehicle groups and N=8 for the pept-in and positive control groups. Graphs show box plots of tumor volumes at day 22 after treatment started. The displayed graphs demonstrate a significant reduction in tumor volume for 04-004-N001 (200 μg dosing group) and 04-015-N001 (20 g and 200 g dosing groups) by one-way ANOVA.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology. That said, as regards the term “consisting essentially of”, by means of further illustration, where a molecule is recited to consist essentially of structural elements A-B-C, the molecule would necessarily include the listed elements and would be open to also contain unlisted structural elements that do not materially affect the basic and novel properties of the molecule. Hence, where the elements A-B-C were to form the operative part or principle of the molecule, in particular by facilitating the molecule's interaction with or effect on a given target, the term “consisting essentially of” would ensure the presence of said elements A-B-C in the molecule, and would also allow for the presence of unlisted elements which do not materially affect the molecule's interaction with said target.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the present invention, the inventors for the first time disclose and demonstrate the therapeutic potential of molecules which comprise one or more β-aggregating sequences designed to specifically target β-aggregation prone regions (APRs) that arise in mutant human RAS proteins due to certain missense mutations at position 12 or at position 13, such as G12V, G12C, G12A, or G12S RAS mutations, or G13V, G13C, or G13S RAS mutations. The invention in particular takes advantage of the fact that the APR predicted at positions 2-12 of wild-type human RAS is altered, such as C-terminally elongated by one or more amino acids and/or may display increased predicted aggregation propensity, as a consequence of missense mutations of G12 or G13 of wild-type RAS to other residues.

Having recognised the presence of such altered APR profiles in G12 or G13 mutant human RAS proteins, the inventors investigated and presently teach molecules which exploit these differences by specifically targeting the altered APRs in the G12 or G13 mutant RAS proteins, but not the corresponding unaltered APR in wild-type RAS, for an intermolecular β-sheet interaction that allows to downregulate the G12 or G13 mutant RAS proteins. Without wishing to be limited to any hypothesis or theory, the data presented herein suggests that this downregulation is likely due to the ability of the molecules to induce specific co-aggregation with the G12 or G13 mutant RAS proteins, which decreases their solubility, sequesters them into aggregates or inclusion bodies (which may be subject to degradation by cellular machinery), and in effect reduces the amount of the G12 or G13 mutant RAS proteins that remain available for intracellular signalling. It shall be understood that once a molecule induced or commenced the aggregation of its target G12 or G13 mutant RAS protein, the so-aggregated RAS can itself acquire the capacity to facilitate or drive the inclusion of additional soluble G12 or G13 mutant RAS protein into the aggregates, i.e., the existing RAS aggregates can function as ‘seeds’ for further aggregation of the protein and growth of the aggregates. The molecules do not display a comparable or equivalent induction of co-aggregation with and downregulation of wild-type RAS. This may for instance mean that even if some intermolecular β-sheet formation were to occur between the molecules and wild-type RAS, the consequences of this will be comparatively negligible and the molecules will not observably downregulate wild-type RAS or will not downregulate wild-type RAS to an extent where such downregulation would detrimentally diminish intracellular signalling by wild-type RAS.

Accordingly, an aspect provides a non-naturally occurring molecule configured to form an intermolecular beta-sheet with a human RAS protein mutated at position 12 (henceforth, a G12 mutant human RAS protein) or mutated at position 13 (henceforth, a G13 mutant human RAS protein) and substantially not with wild-type human RAS protein. The capacity of such molecule to specifically target the G12 or G13 mutant RAS protein for the intermolecular β-sheet formation may manifest in particular as the molecule's ability to downregulate, decrease solubility of and/or induce aggregation or inclusion body formation of the G12 or G13 mutant RAS protein but substantially not of wild-type RAS, such as for example in an appropriate in vitro, cell culture or in vivo setup.

Whereas the aforementioned definition of the molecules may conveniently and meaningfully focus on the molecular mechanism—selective or preferential formation of intermolecular beta-sheets between the molecules and G12 or G13 mutant human RAS proteins compared to wild-type RAS—that is considered to underlie the observed specific downregulation of G12 or G13 mutant human RAS proteins but not wild-type RAS by the molecules, other alternative definitions may be adopted. For example, one such definition may refer to a non-naturally occurring molecule configured to form an intermolecular beta-sheet with a G12 or G13 mutant human RAS protein, wherein said molecule is able to decrease the solubility or induce the aggregation or inclusion body formation of the G12 or G13 mutant human RAS protein and substantially not of wild-type human RAS protein. Another such definition may read on a non-naturally occurring molecule configured to form an intermolecular beta-sheet with a G12 or G13 mutant human RAS protein, wherein said molecule is able to downregulate or decrease the activity of the G12 or G13 mutant human RAS protein and substantially not wild-type human RAS protein.

Certain molecules embodying the principles of the present invention may also be described as a non-naturally occurring molecule capable of downregulating, decreasing the solubility and/or inducing aggregation or inclusion body formation of a G12 or G13 mutant human RAS protein and substantially not of wild-type human RAS protein, wherein the molecule comprises a β-aggregating sequence directed to a β-aggregation prone region (APR) in the G12 or G13 mutant human RAS protein, wherein said APR comprises the mutated amino acid at position 12 of 13 the mutant human RAS protein. Certain molecules embodying the principles of the present invention may also be described as a non-naturally occurring molecule capable of downregulating, decreasing the solubility and/or inducing aggregation or inclusion body formation of a G12 mutant human RAS protein and substantially not of wild-type human RAS protein, wherein the molecule comprises a β-aggregating sequence comprising at least 6 contiguous amino acids of the amino acid sequence: a) TEYKLVVVGAVGVG (SEQ ID NO: 2); or b) TEYKLVVVGACGVG (SEQ ID NO: 6) or preferably TEYKLVVVGACGV (SEQ ID NO: 3); or c) TEYKLVVVGAAGVG (SEQ ID NO: 7) or preferably TEYKLVVVGAAGV (SEQ ID NO: 4); or d) TEYKLVVVGASGVG (SEQ ID NO: 8) or preferably TEYKLVVVGASGV (SEQ ID NO: 9) or more preferably TEYKLVVVGASG (SEQ ID NO: 5); including the amino acid at position 11 of the respective sequences. Certain molecules embodying the principles of the present invention may also be described as a non-naturally occurring molecule capable of downregulating, decreasing the solubility and/or inducing aggregation or inclusion body formation of a G13 mutant human RAS protein and substantially not of wild-type human RAS protein, wherein the molecule comprises a β-aggregating sequence comprising at least 6 contiguous amino acids of the amino acid sequence: a) TEYKLVVVGAGCVG (SEQ ID NO: 84) or preferably TEYKLVVVGAGCV (SEQ ID NO: 81); or b) TEYKLVVVGAGVVG (SEQ ID NO: 82); or c) TEYKLVVVGAGSVG (SEQ ID NO: 85) or preferably TEYKLVVVGAGSV (SEQ ID NO: 86) or more preferably TEYKLVVVGAGS (SEQ ID NO: 83); including the amino acid at position 12 of the respective sequences.

Further aspects provide inter alia: any molecule as taught herein for use in medicine; any molecule as taught herein for use in a method of treating a disease caused by or associated with a G12 or G13 mutation in human RAS protein; a method for treating a subject in need thereof, the method comprising administering to the subject a therapeutically effective amount of any molecule as taught herein; as well as a pharmaceutical composition comprising any molecule as taught herein.

The term “non-naturally occurring” generally refers to a material or an entity that is not formed by nature or does not exist in nature. Such non-naturally occurring material or entity may be made, synthesised, semi-synthesised, modified, intervened on or manipulated by man using methods described herein or known in the art. By means of an example, the term when used in relation to a peptide may in particular denote that a peptide of an identical amino acid sequence is not found in nature, or if a peptide of an identical amino acid sequence is present in nature, that the non-naturally occurring peptide comprises one or more additional structural elements such as chemical bonds, modifications or moieties which are not included in and thus distinguish the non-naturally occurring peptide from the naturally occurring counterpart. In certain embodiments, the term when used in relation to a peptide may denote that the amino acid sequence of the non-naturally occurring peptide is not identical to a stretch of contiguous amino acids encompassed by a naturally occurring peptide, polypeptide or protein. For avoidance of doubt, a non-naturally occurring peptide may perfectly contain an amino acid stretch shorter than the whole peptide, wherein the structure of the amino acid stretch including in particular its sequence is identical to a stretch of contiguous amino acids found in a naturally occurring peptide, polypeptide or protein.

In the context of the present disclosure, the phrase “a molecule configured to” intends to encompass any molecule that exhibits the recited outcome or functionality under appropriate circumstances. Hence, the phrase can be seen as synonymous to and interchangeable with phrases such as “a molecule suitable for”, “a molecule having the capacity to”, “a molecule designed to”, “a molecule adapted to”, “a molecule made to”, or “a molecule capable of”.

The terms “beta-sheet”, “beta-pleated sheet”, “β-sheet”, “β-pleated sheet” are well-known in the art and by virtue of additional explanation interchangeably refer to a molecular structure comprising two or more beta-strands connected laterally by backbone hydrogen bonds (interstrand hydrogen bonding). A beta-strand is a stretch of amino acids typically 3 to 10 amino acids long with backbone in an almost fully extended conformation, following a ‘zigzag’ trajectory. Adjacent amino acid chains in a beta-sheet can run in opposite directions (antiparallel β sheet) or in the same direction (parallel β sheet) or may show a mixed arrangement. When not forming a beta-sheet (e.g., prior to participating in a beta-sheet), the stretch of amino acids may exhibit a non-beta-strand conformation; for example it may have an unstructured conformation.

An “intermolecular” beta-sheet involves beta-strands from two or more separate molecules, such as from two or more separate peptides or peptide-containing molecules, polypeptides and/or proteins. In the context of the instant disclosure, the term particularly denotes a beta-sheet involving one or more beta-strands from one or more molecules as taught herein and one or more beta-strands from one or more G12 or G13 mutant human RAS protein molecules. Given that co-aggregation seeded by the intermolecular beta-sheet formation is considered to play an important role in the mode of action of the present molecules, many tens, hundreds, thousands, or more molecules as taught herein and molecules of G12 or G13 mutant human RAS protein may be involved in underlying beta-sheets interactions, leading to higher order organisation and structures, such as protofibrils, fibrils and aggregates.

Typically, a beta-strand may be formed by only a part of (e.g., by a stretch of contiguous amino acids of) a molecule, peptide, polypeptide or protein that participates in a beta-sheet. For example, the molecule as taught herein may include one or more stretches of contiguous amino acids which become organised into beta-strands participating in beta-sheets in cooperation with one or more beta-strands constituted by stretches of contiguous amino acids of one or more G12 or G13 mutant human RAS protein molecules. In other words, a statement that a molecule can form and intermolecular beta-sheet with a G12 or G13 mutant human RAS protein will typically mean that one or more portions of the molecule, such as one or more stretches of contiguous amino acids of the molecule, is or are designed to organise into beta-strands that can participate in a beta-sheet together with one or more stretches of contiguous amino acids of a G12 or G13 mutant human RAS protein molecule. The interlocking of beta-strands from two or more separate molecules into beta sheets can thus create a complex in which the two or more separate molecules become physically associated or connected and spatially adjacent. In view of the aforementioned explanations, the phrase “a molecule configured to form an intermolecular beta-sheet with a G12 or G13 mutant human RAS protein” may also subsume the meanings: a molecule capable of participating in or contributing to or inducing the generation of an intermolecular beta-sheet with a stretch of contiguous amino acids of a G12 or G13 mutant human RAS protein; a molecule comprising a portion capable of participating in or contributing to or inducing the generation of an intermolecular beta-sheet with a stretch of contiguous amino acids of a G12 or G13 mutant human RAS protein; and a molecule comprising a stretch of contiguous amino acids capable of participating in or contributing to or inducing the generation of an intermolecular beta-sheet with a stretch of contiguous amino acids of a G12 or G13 mutant human RAS protein.

RAS proteins belong to small GTPase class of proteins and have been well-studied in the art. Three human RAS genes have been described: Kirsten rat sarcoma viral oncogene homolog (KRAS) (annotated under U.S. government's National Center for Biotechnology Information (NCBI) Genbank (http://www.ncbi.nlm.nih.gov/) Gene ID no. 3845), neuroblastoma RAS viral oncogene homolog (NRAS) (Gene ID no. 4893), and Harvey rat sarcoma viral oncogene homolog (HRAS) (Gene ID no. 3265). Alternative RNA splicing of the KRAS transcript leads to two known KRAS isoforms, KRAS4A and KRAS4B, that differ in the C-terminal region.

A human wild-type KRAS4A isoform amino acid sequence may be as annotated under Genbank accession no: NP_203524.1 or Swissprot/Uniprot (http://www.uniprot.org/) accession no: P01116-1 (v1), the NP_203524.1 sequence reproduced here below:

(SEQ ID NO: 19)
MTEYKLVVVGAGGVGKSALTIQLIQNHFVDEYDPT
IEDSYRKQVVIDGETCLLDILDTAGQEEYSAMRDQ
YMRTGEGFLCVFAINNTKSFEDIHHYREQIKRVKD
SEDVPMVLVGNKCDLPSRTVDTKQAQDLARSYGIP
FIETSAKTRQRVEDAFYTLVREIRQYRLKKISKEE
KTPGCVKIKKCIIM

A human wild-type KRAS4B isoform amino acid sequence may be as annotated under Genbank accession no: NP_004976.2 or Swissprot/Uniprot accession no: P01116-2 (v1), the NP_004976.2 sequence reproduced here below:

(SEQ ID NO: 20)
MTEYKLVVVGAGGVGKSALTIQLIQNHFVDEYDPT
IEDSYRKQVVIDGETCLLDILDTAGQEEYSAMRDQ
YMRTGEGFLCVFAINNTKSFEDIHHYREQIKRVKD
SEDVPMVLVGNKCDLPSRTVDTKQAQDLARSYGIP
FIETSAKTRQGVDDAFYTLVREIRKHKEKMSKDGK
KKKKKSKTKCVIM

A human wild-type NRAS amino acid sequence may be as annotated under Genbank accession no: NP_002515.1 or Swissprot/Uniprot accession no: P01111 (v1), the NP_002515.1 sequence reproduced here below:

(SEQ ID NO: 21)
MTEYKLVVVGAGGVGKSALTIQLIQNHFVDEYDPT
IEDSYRKQVVIDGETCLLDILDTAGQEEYSAMRDQ
YMRTGEGFLCVFAINNSKSFADINLYREQIKRVKD
SDDVPMVLVGNKCDLPTRTVDTKQAHELAKSYGIP
FIETSAKTRQGVEDAFYTLVREIRQYRMKKLNSSD
DGTQGCMGLPCVVM.

A human wild-type HRAS amino acid sequence may be as annotated under Genbank accession no: NP_005334.1 or Swissprot/Uniprot accession no: P01112 (v1), the NP_005334.1 sequence reproduced here below:

(SEQ ID NO: 22)
MTEYKLVVVGAGGVGKSALTIQLIQNHFVDEYDPTIE
DSYRKQVVIDGETCLLDILDTAGQEEYSAMRDQYMRT
GEGFLCVFAINNTKSFEDIHQYREQIKRVKDSDDVPM
VLVGNKCDLAARTVESRQAQDLARSYGIPYIETSAKT
RQGVEDAFYTLVREIRQHKLRKLNPPDESGPGCMSCK
CVLS.

Hence, in certain embodiments, the RAS protein may be KRAS, NRAS or HRAS protein. In certain preferred embodiments, the RAS protein may be KRAS protein. In human cancers, KRAS is the predominantly mutated RAS isoform (85%). Notably, the amino acid sequences of human KRAS, NRAS and HRAS are basically identical at positions 1-86, which includes the region around G12 and G13, and thus an identical molecule can be used to target G12 or G13 mutant human KRAS, NRAS and HRAS proteins.

The qualifier “human” as used herein in connection with a RAS protein may in a certain interpretation refer to the amino acid sequence of the RAS protein. For example, a RAS protein having the amino acid sequence as a RAS protein found in humans may also be obtained by technical means, e.g., by recombinant expression, cell-free translation, or non-biological peptide synthesis. Because the present molecules are intended to therapeutically target mutant RAS proteins in humans, in a certain other interpretation the qualifier “human” may more particularly refer to a RAS protein as found in or present in humans, regardless of whether the RAS protein forms a part of or has been at least partly isolated from human subjects, organs, cells, or tissues. A skilled person understands that the amino acid sequence of a given native protein such as a RAS protein may differ between or within different individuals of the same species due to normal genetic diversity (allelic variation, polymorphism) within that species and/or due to differences in post-transcriptional or post-translational modifications. Any such variants or isoforms of the native protein are subsumed by the reference to or designation of the protein.

The molecules as taught herein selectively target G12 or G13 mutant human RAS proteins for downregulation through inducing intermolecular beta-sheet formation, but do not substantially alter the amount or biological activity of wild-type RAS. From this perspective, the term “wild-type” may be ascribed the conventional meaning of the RAS variant encoded by the allele of the respective RAS gene that is most commonly observed in a human population. The term “wild-type” may also be given a phenotype-oriented meaning of any RAS variant that is not causative of or associated with a proliferative or neoplastic disease, or a molecular mechanism-oriented meaning of any RAS variant that is not constitutively active, more particularly that is not defective in GAP-mediated GTP hydrolysis. An immediately perceivable distinction between a G12 or G13 mutant human RAS protein and a wild-type human RAS protein is the presence of glycine residues at positions 12 and 13 of the latter. Hence, for example, a wild-type RAS protein may display the sequence GA [symbol] GV (SEQ ID NO: 23) at positions 10-14, or VVGA [symbol] GVGK (SEQ ID NO: 24) at positions 8-16, or LVVVGA [symbol] GVGKSA (SEQ ID NO: 25) at positions 6-18, G12 being shown in bold. Hence, for example, a wild-type RAS protein may display the sequence AG [symbol] VG (SEQ ID NO: 87) at positions 11-15, or VGAG [symbol] VGKS (SEQ ID NO: 88) at positions 9-17, or VVVGAG [symbol] VGKSAL (SEQ ID NO: 89) at positions 7-19, G13 being shown in bold.

Conversely, in a “G12 mutant human RAS” as discussed herein the glycine residue at position 12 (G12) has been mutated. Particularly intended are mutant RAS proteins in which G12 has been replaced by exactly one amino acid other than glycine (G12 missense mutant RAS). Missense mutations replacing G12 of human RAS with virtually every other amino acid have been documented in diseases, including G12A, G12D, G12F, G12L, G12P, G12S, G12V, G12Y, G12C, G12E, G121, G12N, G12R, G12T, and G12W missense mutations (Hobbs et al., supra). G12Q, G12H, G12K, and G12M missense mutations are also conceivable. In a “G13 mutant human RAS” as discussed herein the glycine residue at position 13 (G13) has been mutated. Particularly intended are mutant RAS proteins in which G13 has been replaced by exactly one amino acid other than glycine (G13 missense mutant RAS). Missense mutations replacing G13 of human RAS with virtually every other amino acid have been documented in diseases, including G13A, G13D, G13F, G13M, G13P, G13S, G13Y, G13C, G13E, G131, G13N, G13R, and G13V missense mutations (Hobbs et al., supra). G13L, G13W, G13H, G13K, G13Q and G13T missense mutations are also conceivable.

G12 or G13 mutant human RAS proteins, in particular G12 or G13 missense mutants, may thus be causative of or associated with a proliferative or neoplastic disease and/or may result in a constitutively active RAS, more particularly RAS that is defective in GAP-mediated GTP hydrolysis.

As mentioned previously, the inventors realised that certain G12 or G13 missense mutations alter, such as in particular enlarge (more particularly add one or a few amino acids to the C-terminal end of) the β-aggregation prone region (APR) of human RAS which normally includes and is predicted to conclude with G12 in the wild-type RAS protein and/or increase the aggregation propensity of the APR. Because the inclusion of charged amino acids (such as D, E, R, K or H) proline (P) typically disturbs or diminishes the aggregation propensity of an APR, preferably the G12 or G13 mutant human RAS protein is one in which G12 or G13 is replaced by an uncharged amino acid. In certain preferred embodiments, the G12 or G13 mutant human RAS protein may be one in which G12 or G13 is replaced by a hydrophobic amino acid other than proline, such as glycine (G), alanine (A), valine (V), leucine (L), isoleucine (I), phenylalanine (F), methionine (M), and tryptophan (W). In certain embodiments, the G12 or G13 mutant human RAS protein may be one in which G12 or G13 is replaced by a polar amino acid, such as serine (S), threonine (T), cysteine (C), asparagine (N), glutamine (Q), or tyrosine (Y). In certain preferred embodiments, the G12 or G13 mutant human RAS protein may be one that is particularly prevalent in diseases, such as neoplastic diseases, and in which the mutation lengthens the corresponding APR. Hence, in certain preferred embodiments, the G12 mutant human RAS protein may be a G12V, G12C, G12A, or G12S mutant human RAS protein, such as a G12V, G12C, G12A, or G12S mutant human KRAS, NRAS or HRAS protein, preferably a G12V, G12C, G12A, or G12S mutant human KRAS protein. In particularly preferred embodiments, the G12 mutant human RAS protein may be a G12V mutant human RAS protein, such as a G12V mutant human KRAS, NRAS or HRAS protein, preferably a G12V mutant human KRAS protein. The G12V mutation not only lengthens but also increases the predicted aggregation propensity of the corresponding APR. In certain preferred embodiments, the G13 mutant human RAS protein may be a G13V, G13C, or G13S mutant human RAS protein, such as a G13V, G13C, or G13S mutant human KRAS, NRAS or HRAS protein, preferably a G13V, G13C, or G13S mutant human KRAS protein. In particularly preferred embodiments, the G13 mutant human RAS protein may be a G13V mutant human RAS protein, such as a G13V mutant human KRAS, NRAS or HRAS protein, preferably a G13V mutant human KRAS protein. The G13V mutation not only lengthens but also increases the predicted aggregation propensity of the corresponding APR.

The term “protein” generally encompasses macromolecules comprising one or more polypeptide chains The term “polypeptide” generally encompasses linear polymeric chains of amino acid residues linked by peptide bonds. A “peptide bond”, “peptide link” or “amide bond” is a covalent bond formed between two amino acids when the carboxyl group of one amino acid reacts with the amino group of the other amino acid, thereby releasing a molecule of water. Especially when a protein is only composed of a single polypeptide chain, the terms “protein” and “polypeptide” may be used interchangeably to denote such a protein. The terms are not limited to any minimum length of the polypeptide chain Polypeptide chains consisting essentially of or consisting of 50 or less (≤50) amino acids, such as ≤45, ≤40, ≤35, ≤30, ≤25, ≤20, ≤15, ≤10 or ≤5 amino acids may be commonly denoted as a “peptide”. In the context of proteins, polypeptides or peptides, a “sequence” is the order of amino acids in the chain in an amino to carboxyl terminal direction in which residues that neighbour each other in the sequence are contiguous in the primary structure of the protein, polypeptide or peptide. The terms may encompass naturally, recombinantly, semi-synthetically or synthetically produced proteins, polypeptides or peptides. Hence, for example, a protein, polypeptide or peptide can be present in or isolated from nature, e.g., produced or expressed natively or endogenously by a cell or tissue and optionally isolated therefrom; or a protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis. The terms also encompasses proteins, polypeptides or peptides that carry one or more co- or post-expression-type modifications of the polypeptide chain(s), such as, without limitation, glycosylation, lipidation, acetylation, amidation, phosphorylation, sulphonation, methylation, pegylation (covalent attachment of polyethylene glycol typically to the N-terminus or to the side-chain of one or more Lys residues), ubiquitination, sumoylation, cysteinylation, glutathionylation, oxidation of methionine to methionine sulphoxide or methionine sulphone, signal peptide removal, N-terminal Met removal, conversion of pro-enzymes or pre-hormones into active forms, etc. Such co- or post-expression-type modifications may be introduced in vivo by a host cell expressing the proteins, polypeptides or peptides (co- or post-translational protein modification machinery may be native to the host cell and/or the host cell may be genetically engineered to comprise one or more (additional) co- or post-translational protein modification functionalities), or may be introduced in vitro by chemical (e.g., pegylation) and/or biochemical (e.g., enzymatic) modification of the isolated proteins, polypeptides or peptides. By means of an example and without limitation, in certain embodiments acetylation of the free alpha amino group at the N-terminus of chemically synthesized peptides and/or the amidation of the free carboxyl group at the C-terminus of chemically synthesized peptides may be opted for to alter the overall charge of the peptides and/or to stabilize the resulting peptides and enhance their ability to resist enzymatic degradation by exopeptidases.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. A further example of such an amino acid is citrulline. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

The characterisation of the present molecules as being able to form an intermolecular beta-sheet with G12 or G13 mutant human RAS proteins is based inter alia on the mechanisms described in WO 2007/071789A1 and WO2012/123419A1 as underlying the operation of the ‘interferor’ technology. However, the emergence of beta-sheet conformation may also be experimentally assessed by available methods. By means of a non-limiting example, nuclear magnetic resonance (NMR) spectroscopy has been employed for many years to characterise the secondary structure of proteins in solution (reviewed in Wuetrich et al. FEBS Letters. 1991, vol. 285, 237-247).

Perhaps more straightforwardly in the context of the present invention, the formation of the intermolecular beta-sheet leads to an interaction between the molecule and the G12 or G13 mutant human RAS protein, which can be qualitatively and quantitatively assessed by standard methods such as co-immunoprecipitation assays. Several instances of such co-immunoprecipitation assays are presented in the Examples. In one illustrative approach, cells expressing G12 mutant or wild-type RAS were contacted with molecules as taught herein labelled with biotin, the cells were lysed, the molecules (and any RAS proteins bound thereto) were pulled down by streptavidin-coated beads, and the co-precipitated RAS protein was quantified by an immunoassay method, namely a quantitative Western blot. In another illustrative approach, in vitro translation reactions producing G12 mutant or wild-type RAS were contacted with molecules as taught herein labelled with biotin, the molecules (and any RAS proteins bound thereto) were pulled down by streptavidin-coated beads, and the co-precipitated RAS protein was quantified by an immunoassay method, namely a quantitative Western blot. Also in the context of the present invention, the interaction between the molecule and the G12 or G13 mutant human RAS can lead to reduced solubility of RAS and even emergence of aggregates or inclusion bodies containing the RAS protein in cells. This can be analysed by standard immunoassay or fluorescence microscopy methods also exemplified in the Examples. In one illustrative approach, cells expressing G12 mutant or wild-type RAS were contacted with molecules as taught herein, the cells were lysed by a non-denaturing buffer and proteins insoluble in this buffer were treated with a strong chaotropic agent (6M urea). RAS present in the fraction remaining insoluble after this treatment was quantified by an immunoassay method, namely a quantitative Western blot. In another illustrative approach, cultured mammalian such as human cells were transfected with G12 mutant or wild-type RAS fused to a fluorescent moiety, such as a standard green or red fluorescent protein, the cells were treated with molecules as taught herein and the cellular localization of the fluorescently-tagged RAS was determined by fluorescence microscopy. These illustrative assays, which can be applied and adopted according to circumstances, have the advantage that the molecules can contact RAS when the latter is being produced on ribosomes (in cells or in vitro). In such not-yet-folded RAS the targeted APR is expected to be comparatively more accessible and exposed to the environment, which can facilitate the intermolecular interaction with the molecules. Further in the context of the present invention, the interaction between the molecule and the G12 or G13 mutant human RAS is intended to downregulate the mutant RAS, which can be detected and quantified for example by measuring the reduction in viability of transformed cell lines that depend for their growth on constitutive RAS signalling by a G12 or G13 mutant RAS, when exposed to molecules as taught herein. One such exemplary cell line for G12 mutant RAS is NCI-H441 lung adenocarcinoma cells, obtainable inter alia from American Type Culture Collection (ATCC) (10801 University Blvd. Manassas, Va. 20110-2209, USA), accession no. HTB-174™. This is also illustrated in the Examples.

The description of the present molecules as substantially not forming intermolecular beta-sheets with wild-type human RAS may in particular convey that the extent to which the molecules might downregulate signalling by human wild-type RAS, if at all, is negligible or insignificant compared to the extent to which they downregulate signalling by their respective G12 or G13 mutant human RAS. For example, RAS signalling may be assessed in cultured cells expressing wild-type RAS exposed to external stimuli known to stimulate downstream pathways involving RAS. In practice, the molecules when administered in therapeutically effective and realistic quantities would cause no or only minor or tolerable undesired effects attributable to downregulation of normal RAS signalling in cells expressing only wild-type RAS. Where assays or tests as described above, such as in vitro assays or tests performed in cultured cells, e.g., molecule-RAS co-immunoprecipitation assays, RAS solubility measurements, or fluorescence microscopy assays to visualise aggregates of

RAS, are used to assess the formation of the intermolecular beta-sheet, the substantial lack of intermolecular beta-sheet formation between the molecules and wild-type RAS may be observed as the absence of a signal (i.e., the absence of an outcome or measurement considered ‘positive’) in the respective assays, or as the presence of a quantifiable signal that is comparable to or not significantly higher than a signal produced by a negative control (e.g., by a molecule of a similar chemical composition but without any or with only negligible beta-sheet forming quality, e.g., by a scrambled peptide in case of peptide molecules), or as the presence of a quantifiable signal that is considerably lower or less intense than the signal produced by the molecule for G12 or G13 mutant RAS. For example, the signal (e.g., the quantity of RAS co-precipitated with a molecule, the quantity insoluble RAS or the proportion of insoluble vs. soluble RAS, or the number, size or fluorescence intensity of visible RAS aggregates in cells) produced by a molecule for wild-type RAS may be, in order of increasing preference, at least 10-fold lower, at least 10²-fold lower, at least 10³-fold lower, at least 10⁴-fold lower, at least 10⁵-fold lower, or at least 10⁶-fold lower than the signal produced by the molecule for G12 or G13 mutant RAS.

As explained earlier, certain missense mutations at position 12 or 13 of wild-type human RAS alter the APR predicted at positions 2-12 of wild-type human RAS, such that this altered APR will include the mutated amino acid at position 12 and will typically further include one or a few amino acids C-terminally adjacent to this mutated amino acid, or such that this altered APR will include the mutated amino acid at position 13 and may also further include one or a few amino acids C-terminally adjacent to this mutated amino acid. Hence, the APR predicted in the G12 mutant RAS may span positions 2-12, or preferably positions 2-13, 2-14 or 2-15, or more preferably positions 2- 14 or 2-15, or even more preferably positions 2-15 of the G12 mutant RAS. The APR predicted in the G13 mutant RAS may span positions 2-13, or preferably positions 2-14 or 2-15, or even more preferably positions 2-15 of the G13 mutant RAS.

As also stated earlier, beta-strands tend to be 3 to 10 amino acids long. Accordingly, in certain embodiments the intermolecular beta-sheet formed between the molecule and its target G12 or G13 mutant human RAS may involve at least 3, such as at least 4 or at least 5, contiguous amino acids of the N-terminal most APR predicted in the mutant RAS, in particular of the APR predicted at positions 2-12, 2-13, 2-14 or 2-15 of the G12 mutant RAS, or of the APR predicted at positions 2-13, 2-14 or 2-15 of the G13 mutant RAS. Put differently, said at least 3, at least 4 or at least 5 contiguous amino acids of the mutant RAS will constitute a beta-strand that participates in the beta-sheet. To enhance specificity of the targeting, the molecules may be designed such as to induced beta-sheets that involve at least 6, such as exactly 6, or at least 7, such as exactly 7, or at least 8, such as exactly 8, or at least 9, such as exactly 9, or at least 10, such as exactly 10, contiguous amino acids of the N-terminal most APR predicted in the mutant RAS, in particular of the APR predicted at positions 2-12, 2-13, 2-14 or 2-15 of the G12 mutant RAS, or of the APR predicted at positions 2-13, 2-14 or 2-15 of the G13 mutant RAS. Beta-sheets involving 11, 12, 13 or 14 contiguous amino acids of said APR are also conceivable, even though beta-strands of 6 to 10 contiguous amino acids may be preferred, since they allow for satisfactory specificity while simplifying the design of the molecules.

Further, in certain embodiments, the beta-sheet induced by the molecule will involve the amino acid at position 12 of the targeted G12 mutant human RAS protein. Put differently, the amino acid at position 12 will be part of a beta-strand that participates in the beta-sheet. Where the mutation at position 12 of RAS results in a predicted APR that extends beyond position 12, the beta-sheet may preferably additionally involve at least one amino acid that is C-terminal to position 12. By means of illustrative embodiments: where the mutation at position 12 of RAS results in a predicted APR that spans positions 2-12 of the G12 mutant RAS, the beta-sheet may involve the amino acid at position 12 of the mutant RAS; where the mutation at position 12 of RAS results in a predicted APR that spans positions 2-13 of the G12 mutant RAS, the beta-sheet may involve the amino acid at position 12 of the mutant RAS or may involve the amino acids at positions 12 and 13 of the mutant RAS; where the mutation at position 12 of RAS results in a predicted APR that spans positions 2-14 of the G12 mutant RAS, the beta-sheet may involve the amino acid at position 12 of the mutant RAS or may involve the amino acids at positions 12 and 13 of the mutant RAS or may involve the amino acids at positions 12 to 14 of the mutant RAS; or where the mutation at position 12 of RAS results in a predicted APR that spans positions 2-15 of the G12 mutant RAS, the beta-sheet may involve the amino acid at position 12 of the mutant RAS or may involve the amino acids at positions 12 and 13 of the mutant RAS or may involve the amino acids at positions 12 to 14 of the mutant RAS or may involve the amino acids at positions 12 to 15 of the mutant RAS.

In such embodiments, the beta-sheet will typically additionally involve one or more contiguous amino acids N-terminally adjacent to the amino acid at position 12 of the G12 mutant RAS, whereby the beta-sheet may thus involve at least 6, such as 6 to 10, contiguous amino acids of the APR. By means of illustrative embodiments: the beta-sheet may involve the amino acid at position 12 of the G12 mutant RAS and at least 5, such as 5 to 9, contiguous amino acids N-terminally adjacent to the amino acid at position 12 of the mutant RAS; or the beta-sheet may involve the amino acids at positions 12 and 13 of the G12 mutant RAS and at least 4, such as 4 to 8, contiguous amino acids N-terminally adjacent to the amino acid at position 12 of the mutant RAS; or the beta-sheet may involve the amino acids at positions 12 to 14 of the G12 mutant RAS and at least 3, such as 3 to 7, contiguous amino acids N-terminally adjacent to the amino acid at position 12 of the mutant RAS; or the beta-sheet may involve the amino acids at positions 12 to 15 of the G12 mutant RAS and at least 2, such as 2 to 6, contiguous amino acids N-terminally adjacent to the amino acid at position 12 of the mutant RAS.

The sequence of the N-terminal most APR predicted at positions 2-12 of human wild-type RAS, TEYKLVVVGAG (SEQ ID NO: 1), includes two charged residues, glutamate (E) at position 3 of RAS and lysine (K) at position 5 of RAS. Whereas the portion of the APR containing these charged residues may also be targeted for beta-sheet formation, the ‘interferon’ technology as described in WO 2007/071789A1 and WO2012/123419A1 preferably targets amino acid sequences predominantly composed of uncharged amino acids. Hence, in certain preferred embodiments, the portion of the APR N-terminally demarcated by the leucine (L) at position 6 of RAS may be targeted. Accordingly, by means of illustrative embodiments: the beta-sheet may involve the amino acids at positions 6-12 or 6-13 or 6-14 or 6-15, or 7-12 or 7-13 or 7-14 or 7-15, or 8-13 or 8-14 or 8-15, or 9-14 or 9-15, or 10-15 of the mutant RAS.

Accordingly, in certain embodiments, the intermolecular beta-sheet may involve a portion of, in particular a contiguous portion of, or the whole of the amino acid sequence TEYKLVVVGAVGVG (SEQ ID NO: 2) in the G12V mutant human RAS protein, such as for example at least 6, at least 7, at least 8, at least 9 or at least 10 contiguous amino acids of SEQ ID NO: 2. In certain preferred embodiments, the intermolecular beta-sheet may involve a portion of, in particular a contiguous portion of, or the whole of the amino acid sequence LVVVGAVGVG (SEQ ID NO: 26) in the G12V mutant human RAS protein, such as for example at least 6, at least 7, at least 8, at least 9 or all 10 contiguous amino acids of SEQ ID NO: 26.

In certain embodiments, the intermolecular beta-sheet may involve a portion of, in particular a contiguous portion of, or the whole of the amino acid sequence TEYKLVVVGACGVG (SEQ ID NO: 6) or preferably TEYKLVVVGACGV (SEQ ID NO: 3) in the G12C mutant human RAS protein, such as for example at least 6, at least 7, at least 8, at least 9 or at least 10 contiguous amino acids of SEQ ID NO: 6 or 3. In certain preferred embodiments, the intermolecular beta-sheet may involve a portion of, in particular a contiguous portion of, or the whole of the amino acid sequence LVVVGACGVG (SEQ ID NO: 27) or preferably LVVVGACGV (SEQ ID NO: 28) in the G12C mutant human RAS protein, such as for example at least 6, at least 7, at least 8, at least 9 or all 10 contiguous amino acids of SEQ ID NO: 27, or at least 6, at least 7, at least 8 or all 9 contiguous amino acids of SEQ ID NO: 28.

In certain embodiments, the intermolecular beta-sheet may involve a portion of, in particular a contiguous portion of, or the whole of the amino acid sequence TEYKLVVVGAAGVG (SEQ ID NO: 7) or preferably TEYKLVVVGAAGV (SEQ ID NO: 4) in the G12A mutant human RAS protein, such as for example at least 6, at least 7, at least 8, at least 9 or at least 10 contiguous amino acids of SEQ ID NO: 7 or 4. In certain preferred embodiments, the intermolecular beta-sheet may involve a portion of, in particular a contiguous portion of, or the whole of the amino acid sequence LVVVGAAGVG (SEQ ID NO: 29) or preferably LVVVGAAGV (SEQ ID NO: 30) in the G12A mutant human RAS protein, such as for example at least 6, at least 7, at least 8, at least 9 or all 10 contiguous amino acids of SEQ ID NO: 29, or at least 6, at least 7, at least 8 or all 9 contiguous amino acids of SEQ ID NO: 30.

In certain embodiments, the intermolecular beta-sheet may involve a portion of, in particular a contiguous portion of, or the whole of the amino acid sequence TEYKLVVVGASGVG (SEQ ID NO: 8) or preferably TEYKLVVVGASGV (SEQ ID NO: 9) or more preferably TEYKLVVVGASG (SEQ ID NO: 5) in the G12S mutant human RAS protein, such as for example at least 6, at least 7, at least 8, at least 9 or at least 10 contiguous amino acids of SEQ ID NO: 8 or 9 or 5. In certain preferred embodiments, the intermolecular beta-sheet may involve a portion of, in particular a contiguous portion of, or the whole of the amino acid sequence LVVVGASGVG (SEQ ID NO: 31) or preferably LVVVGASGV (SEQ ID NO: 32) or more preferably LVVVGASG (SEQ ID NO: 33) in the G12S mutant human RAS protein, such as for example at least 6, at least 7, at least 8, at least 9 or all 10 contiguous amino acids of SEQ ID NO: 31, or at least 6, at least 7, at least 8 or all 9 contiguous amino acids of SEQ ID NO: 32, or at least 6, at least 7 or all 8 contiguous amino acids of SEQ ID NO: 33 in the G12S mutant human RAS protein.

Further, in certain embodiments, the beta-sheet induced by the molecule will involve the amino acid at position 13 of the targeted G13 mutant human RAS protein. Put differently, the amino acid at position 13 will be part of a beta-strand that participates in the beta-sheet. Where the mutation at position 13 of RAS results in a predicted APR that extends beyond position 13, the beta-sheet may preferably additionally involve at least one amino acid that is C-terminal to position 13. By means of illustrative embodiments: where the mutation at position 13 of RAS results in a predicted APR that spans positions 2-13 of the G13 mutant RAS, the beta-sheet may involve the amino acid at position 13 of the mutant RAS; where the mutation at position 13 of RAS results in a predicted APR that spans positions 2-14 of the G13 mutant RAS, the beta-sheet may involve the amino acid at position 13 of the mutant RAS or may involve the amino acids at positions 13 and 14 of the mutant RAS; where the mutation at position 13 of RAS results in a predicted APR that spans positions 2-15 of the G13 mutant RAS, the beta-sheet may involve the amino acid at position 13 of the mutant RAS or may involve the amino acids at positions 13 and 14 of the mutant RAS or may involve the amino acids at positions 13 to 15 of the mutant RAS.

In such embodiments, the beta-sheet will typically additionally involve one or more contiguous amino acids N-terminally adjacent to the amino acid at position 13 of the mutant RAS, whereby the beta-sheet may thus involve at least 6, such as 6 to 10, contiguous amino acids of the APR. By means of illustrative embodiments: the beta-sheet may involve the amino acid at position 13 of the G13 mutant RAS and at least 5, such as 5 to 9, contiguous amino acids N-terminally adjacent to the amino acid at position 13 of the mutant RAS; or the beta-sheet may involve the amino acids at positions 13 and 14 of the G13 mutant RAS and at least 4, such as 4 to 8, contiguous amino acids N-terminally adjacent to the amino acid at position 13 of the mutant RAS; or the beta-sheet may involve the amino acids at positions 13 to 15 of the G13 mutant RAS and at least 3, such as 3 to 7, contiguous amino acids N-terminally adjacent to the amino acid at position 13 of the mutant RAS.

In certain embodiments, the intermolecular beta-sheet may involve a portion of, in particular a contiguous portion of, or the whole of the amino acid sequence TEYKLVVVGAGVVG (SEQ ID NO: 82) in the G13V mutant human RAS protein, such as for example at least 6, at least 7, at least 8, at least 9 or at least 10 contiguous amino acids of SEQ ID NO: 82. In certain preferred embodiments, the intermolecular beta-sheet may involve a portion of, in particular a contiguous portion of, or the whole of the amino acid sequence LVVVGAGVVG (SEQ ID NO: 90) in the G13V mutant human RAS protein, such as for example at least 6, at least 7, at least 8, at least 9 or all 10 contiguous amino acids of SEQ ID NO: 90.

In certain embodiments, the intermolecular beta-sheet may involve a portion of, in particular a contiguous portion of, or the whole of the amino acid sequence TEYKLVVVGAGCVG (SEQ ID NO: 91) or preferably TEYKLVVVGAGCV (SEQ ID NO: 81) in the G13C mutant human RAS protein, such as for example at least 6, at least 7, at least 8, at least 9 or at least 10 contiguous amino acids of SEQ ID NO: 91 or 81. In certain preferred embodiments, the intermolecular beta-sheet may involve a portion of, in particular a contiguous portion of, or the whole of the amino acid sequence LVVVGAGCVG (SEQ ID NO: 92) or preferably LVVVGAGCV (SEQ ID NO: 93) in the G12C mutant human RAS protein, such as for example at least 6, at least 7, at least 8, at least 9 or all 10 contiguous amino acids of SEQ ID NO: 92, or at least 6, at least 7, at least 8 or all 9 contiguous amino acids of SEQ ID NO: 93.

In certain embodiments, the intermolecular beta-sheet may involve a portion of, in particular a contiguous portion of, or the whole of the amino acid sequence TEYKLVVVGAGSVG (SEQ ID NO: 94) or preferably TEYKLVVVGAGSV (SEQ ID NO: 95) or more preferably TEYKLVVVGAGS (SEQ ID NO: 83) in the G13S mutant human RAS protein, such as for example at least 6, at least 7, at least 8, at least 9 or at least 10 contiguous amino acids of SEQ ID NO: 94, 95 or 83. In certain preferred embodiments, the intermolecular beta-sheet may involve a portion of, in particular a contiguous portion of, or the whole of the amino acid sequence LVVVGAGSVG (SEQ ID NO: 96) or preferably LVVVGAGSV (SEQ ID NO: 97) or more preferably LVVVGAGS (SEQ ID NO: 98) in the G13S mutant human RAS protein, such as for example at least 6, at least 7, at least 8, at least 9 or all 10 contiguous amino acids of SEQ ID NO: 96, or at least 6, at least 7, at least 8 or all 9 contiguous amino acids of SEQ ID NO: 97, or at least 6, at least 7 or all 8 contiguous amino acids of SEQ ID NO: 98.

As described, the present molecules are designed to induce intermolecular β-sheet formation with their respective target G12 or G13 mutant RAS proteins, leading to specific downregulation or knock-down of the latter. Based on experimental observations, the molecules can bring about reduced solubility and aggregation of the targeted mutant RAS. Hence, in certain embodiments, the molecules as taught herein are able to decrease the solubility and/or to induce the aggregation or inclusion body formation of their targeted G12 or G13 mutant human RAS protein. Suitable assays to assess RAS solubility and aggregation are discussed elsewhere in this specification.

Any meaningful extent of downregulation of the activity of the G12 or G13 mutant RAS is envisaged. Hence, the terms “downregulate” or “downregulated”, or “reduce” or “reduced”, or “decrease” or “decreased” may in appropriate contexts, such as in experimental or therapeutic contexts, denote a statistically significant decrease relative to a reference. The skilled person is able to select such a reference. An example of a suitable reference may be the G12 or G13 mutant RAS activity when exposed to a ‘negative control’ molecule, such as a molecule of similar composition but known to have no effects on the G12 or G13 mutant RAS. For example, such decrease may fall outside of error margins for the reference (as expressed, for example, by standard deviation or standard error, or by a predetermined multiple thereof, e.g., ±1×SD or ±2×SD, or ±1×SE or ±2×SE). By means of an illustration, the activity of the G12 or G13 mutant RAS may be considered reduced when it is decreased by at least 10%, such as by at least 20% or by at least 30%, preferably by at least 40%, such as by at least 50% or by at least 60%, more preferably by at least 70%, such as by at least 80% or by at least 90% or more, as compared to the reference, up to and including a 100% decrease (i.e., absent activity as compared to the reference).

Any meaningful extent of reduction in solubility of the G12 or G13 mutant RAS is envisaged. This may in appropriate contexts, such as in experimental or therapeutic contexts, denote a statistically significant decrease of the amount of RAS present in the soluble protein fraction, or a statistically significant increase of the amount of RAS present in the insoluble protein fraction, or a statistically significant decrease in the relative abundance of RAS in the soluble vs. insoluble protein fractions, relative to a respective reference. The skilled person is able to select such a reference, such as in particular a reference indicative of G12 or G13 mutant RAS solubility in the presence of a ‘negative control’ molecule. For example, such decrease in solubility may fall outside of error margins for the reference (as expressed, for example, by standard deviation or standard error, or by a predetermined multiple thereof, e.g., ±1×SD or ±2×SD, or ±1×SE or ±2×SE). By means of an illustration, the solubility of the G12 or G13 mutant RAS may be considered reduced when it is decreased by at least 10%, such as by at least 20% or by at least 30%, preferably by at least 40%, such as by at least 50% or by at least 60%, more preferably by at least 70%, such as by at least 80% or by at least 90% or more, as compared to the reference, up to and including a 100% decrease (i.e., no RAS present in the soluble protein fraction/all RAS present in the insoluble protein fraction).

The present molecules are able to induce the formation of an intermolecular beta-sheet with a G12 or G13 mutant human RAS protein, more particularly with the most N-terminal APR predicted in the G12 or G13 mutant human RAS protein. To this end, the molecules may advantageously comprise at least one portion that can assume or mimic a beta-strand conformation capable of interacting with the beta-strand contributed by the RAS protein APR so as to give rise to an intermolecular beta-sheet formed by said interacting beta-strands.

In certain embodiments, the molecule may comprise at least one amino acid stretch which participates in the intermolecular beta-sheet. As explained earlier, beta-strands tend to be 3 to 10 amino acids long. Accordingly, in certain embodiments the at least one amino acid stretch comprised by the molecule may be at least 3, such as at least 4 or at least 5, contiguous amino acids long. To enhance specificity of the interaction, the at least one amino acid stretch comprised by the molecule may be at least 6, such as exactly 6, or at least 7, such as exactly 7, or at least 8, such as exactly 8, or at least 9, such as exactly 9, or at least 10, such as exactly 10, contiguous amino acids long Amino acid stretches that are 11, 12, 13 or 14 contiguous amino acids long can also be conceivably comprised by the molecule, but stretches of 6 to 10 contiguous amino acids may be preferred, since they allow for satisfactory specificity while simplifying the design of the molecules.

In certain preferred embodiments, the at least one stretch of amino acids, such as the at least one stretch of 6 to 10 contiguous amino acids, comprised by the molecule (henceforth “the molecule stretch” for brevity) may correspond to the stretch of contiguous amino acids within the N-terminal most APR of the mutant human RAS which is to participate in the beta-sheet (henceforth “the RAS stretch” for brevity). By means of certain examples, when the beta-sheet is to involve a RAS stretch of 3, 4, 5, preferably 6 to 10, such as 6, 7, 8, 9 or 10, or even 11, 12, 13 or 14 contiguous amino acids of the N-terminal most APR predicted at positions 2-12, 2-13, 2-14 or 2-15 of the mutant human RAS (the APR length depending on the precise G12 or G13 mutation), the molecule stretch can correspond to this RAS stretch. By means of certain further examples, when the beta-sheet is to involve a RAS stretch including the amino acid at position 12 or the amino acids at positions 12-13, 12-14 or 12-15 of the G12 mutant human RAS, or including the amino acid at position 13 or the amino acids at positions 13-14 or 13-15 of the G13 mutant human RAS, the molecule stretch can correspond to this RAS stretch. By means of yet further examples, when the beta-sheet is to involve a RAS stretch at positions 6-12 or 6-13 or 6-14 or 6-15, or 7-12 or 7-13 or 7-14 or 7-15, or 8-13 or 8-14 or 8-15, or 9-14 or 9-15, or 10-15 of the mutant human RAS, the molecule stretch can correspond to this RAS stretch.

The correspondence between the molecule stretch and the RAS stretch may in particular encompass:

a) the situation that the amino acid sequence of the molecule stretch is identical to the amino acid sequence of the RAS stretch;

b) the situation that the amino acid sequence of the molecule stretch is at least 80% identical to the amino acid sequence of the RAS stretch, insofar this degree of sequence identity is compatible with the formation of the intermolecular beta-sheet as taught herein—for example, said at least 80% sequence identity may in certain embodiments denote that when the RAS stretch is 6 or 7 amino acids long the 6 or 7 amino acid-long molecule stretch differs from the RAS stretch by at most 1 amino acid substitution, or when the RAS stretch is 8 to 12 amino acids long the 8 to 12 amino acid-long molecule stretch differs from the RAS stretch by at most 2 amino acid substitutions, or when the RAS stretch is 13 to 14 amino acids long the 13 to 14 amino acid-long molecule stretch differs from the RAS stretch by at most 3 amino acid substitutions;

c) the situation that the amino acid sequence of the molecule stretch differs from the amino acid sequence of the RAS stretch by at most 3, preferably at most 2, and more preferably at most 1 amino acid substitutions, insofar this substitution or substitutions are compatible with the formation of the intermolecular beta-sheet as taught herein;

d) the situation that the amino acid sequence of the molecule stretch displays the degree of sequence identity to the amino acid sequence of the RAS stretch as set forth in any one of a) to c) above, and all amino acids of the molecule stretch are L-amino acids;

e) the situation that the amino acid sequence of the molecule stretch displays the degree of sequence identity to the amino acid sequence of the RAS stretch as set forth in any one of a) to c) above, and at least one (e.g., at least 2, at least 3, at least 4, at least 5, or at least 6 or more or all) amino acid of the molecule stretch is a D-amino acid, insofar the incorporation of the D-amino acid or D-amino acids is compatible with the formation of the intermolecular beta-sheet as taught herein;

f) the situation that the amino acid sequence of the molecule stretch displays the degree of sequence identity to the amino acid sequence of the RAS stretch as set forth in any one of a) to c) above, and at least one (e.g., at least 2, at least 3, at least 4, at least 5, or at least 6 or more or all) amino acid of the molecule stretch is replaced by an analogue of the respective amino acid, insofar the incorporation of the analogue or analogues is compatible with the formation of the intermolecular beta-sheet as taught herein; or

g) the situation that the amino acid sequence of the molecule stretch displays the degree of sequence identity to the amino acid sequence of the RAS stretch as set forth in any one of a) to c) above, and at least one amino acid of the molecule stretch is a D-amino acid and at least one amino acid of the molecule stretch is replaced by an analogue of the respective amino acid, insofar the incorporation of the D-amino acid or D-amino acids and the analogue or analogues is compatible with the formation of the intermolecular beta-sheet as taught herein.

Preferably, the molecule stretch may be designed such that its amino acid sequence is not identical to an amino acid sequence in human proteins other than the RAS family members, to reduce or prevent off-target activity of molecules containing such molecule stretch. The amino acid sequence of the molecule stretch can be readily aligned with the full human proteome to perform this assessment.

The term “sequence identity” with regard to amino acid sequences denotes the extent of overall sequence identity (i.e., including the whole or entire amino acid sequences in the comparison) expressed in % between the amino acid sequences read from N-terminus to C-terminus. Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62 (Henikoff et al., 1992, Proc. Natl. Acad. Sci., 89:10915-10919), cost to open a gap=11, cost to extend a gap=1, expectation value=10.0, word size=3).

An example procedure to determine the percent identity between a particular amino acid sequence and a query amino acid sequence (e.g., the sequence of a RAS stretch) will entail aligning the two amino acid sequences each read from N-terminus to C-terminus using the Blast 2 sequences (B12seq) algorithm, available as a web application or as a standalone executable programme (BLAST version 2.2.31+) at the NCBI web site (www.ncbi.nlm.nih.gov), using suitable algorithm parameters. An example of suitable algorithm parameters includes: matrix=Blosum62, cost to open a gap=11, cost to extend a gap=1, expectation value=10.0, word size=3). If the two compared sequences share identity, then the output will present those regions of identity as aligned sequences. If the two compared sequences do not share identity, then the output will not present aligned sequences. Once aligned, the number of matches will be determined by counting the number of positions where an identical amino acid residue is presented in both sequences. The percent identity is determined by dividing the number of matches by the length of the query sequence, followed by multiplying the resulting value by 100. The percent identity value may, but need not, be rounded to the nearest tenth. For example, 78.11, 78.12, 78.13, and 78.14 may be rounded down to 78.1, while 78.15, 78.16, 78.17, 78.18, and 78.19 may be rounded up to 78.2. It is further noted that the detailed view for each segment of alignment as outputted by Bl2seq already conveniently includes the percentage of identities.

As mentioned, in certain embodiments the amino acid sequence of the molecule stretch may be less than 100% identical to the amino acid sequence of the RAS stretch, for example, the molecule stretch sequence may be at least 80%, e.g., 81%, 82%, 83%, or 84%, preferably at least 85%, e.g., 86%, 87%, 88%, or 89%, more preferably at least 90%, e.g., 91%, 92%, 93%, 94%, 95%, 96%, 97%, 98%, or 99%, identical to the RAS stretch sequence.

In such embodiments, the molecule stretch may comprise one or more amino acid additions, deletions, or substitutions relative to (i.e., compared with) the RAS stretch. Preferably, the molecule stretch may comprise one or more amino acid substitutions, preferably at most 3 or more preferably at most 2 or even more preferably at most 1 amino acid substitution, such as in particular one or more single amino acid substitutions, preferably at most 3 or more preferably at most 2 or even more preferably at most 1 single amino acid substitution, relative to the RAS stretch.

Preferably, the one or more amino acid substitutions, in particular the one or more single amino acid substitutions may be conservative amino acid substitutions. A conservative amino acid substitution is a substitution of one amino acid for another with similar characteristics. Conservative amino acid substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (i.e., basic) amino acids include arginine, lysine and histidine. The negatively charged (i.e., acidic) amino acids include aspartic acid and glutamic acid. Any substitution of one member of the above-mentioned polar, basic, or acidic groups by another member of the same group can be deemed a conservative substitution. By contrast, a non-conservative substitution is a substitution of one amino acid for another with dissimilar characteristics.

In certain embodiments, the one or more amino acid substitutions, in particular the one or more single amino acid substitutions, may each independently be with an uncharged amino acid, preferably with a hydrophobic amino acid other than proline, such as with glycine (G), alanine (A), valine (V), leucine (L), isoleucine (I), phenylalanine (F), methionine (M), and tryptophan (W). Such substitutions can increase the beta-sheet inducing potential of the molecule stretch.

In certain preferred embodiments, the amino acid of the molecule stretch that corresponds to or aligns with position 12 of the targeted G12 mutant human RAS may be identical to, or may be a D-isomer of or may be an analogue of, preferably is identical to, the amino acid found at position 12 of the mutant RAS. By means of examples:

in a molecule directed to G12V mutant human RAS, the amino acid of the molecule stretch that corresponds to position 12 of the G12V RAS would be L-valine or D-valine or a valine analogue, preferably L-valine; in a molecule directed to G12A mutant human RAS, the amino acid of the molecule stretch that corresponds to position 12 of the G12A RAS would be L-alanine or D-alanine or an alanine analogue, preferably L-alanine; in a molecule directed to G12S mutant human RAS, the amino acid of the molecule stretch that corresponds to position 12 of the G12S RAS would be L-serine or D-serine or a serine analogue, preferably L-serine; or in a molecule directed to G12C mutant human RAS, the amino acid of the molecule stretch that corresponds to position 12 of the G12C RAS would be L-cysteine or D-cysteine or a cysteine analogue, preferably L-cysteine. In connection with G12C RAS, the inclusion of an unprotected cysteine in the molecule may be less opportune due to the presence of the reactive —SH group in the cysteine residue. Accordingly, molecules directed against G12C RAS may contain another amino acid, such as serine, at that position, or may contain a cysteine at that position that is otherwise protected, for example by a protective group (e.g., a p-methylbenzyl group, a diphenylmethyl group, a p-methoxybenzyl group, or an acetamidomethyl group), or by reacting its —SH group with the —SH group of another cysteine in the same molecule or between two molecules (disulphide bridge). Hence, in certain embodiments, in a molecule directed to G12C mutant human RAS, the amino acid of the molecule stretch that corresponds to position 12 of the G12C RAS would be L-serine or D-serine or a serine analogue, preferably L-serine. In certain other embodiments, in a molecule directed to G12C mutant human RAS, the amino acid of the molecule stretch that corresponds to position 12 of the G12C RAS would be L-cysteine or D-cysteine or a cysteine analogue, preferably L-cysteine, having its —SH group protected by a protective group or participating in a disulphide bridge.

In certain preferred embodiments, the amino acid of the molecule stretch that corresponds to or aligns with position 13 of the targeted G13 mutant human RAS may be identical to, or may be a D-isomer of or may be an analogue of, preferably is identical to, the amino acid found at position 13 of the mutant RAS. By means of examples: in a molecule directed to G13V mutant human RAS, the amino acid of the molecule stretch that corresponds to position 13 of the G13V RAS would be L-valine or D-valine or a valine analogue, preferably L-valine; in a molecule directed to G13S mutant human RAS, the amino acid of the molecule stretch that corresponds to position 13 of the G13S RAS would be L-serine or D-serine or a serine analogue, preferably L-serine; or in a molecule directed to G13C mutant human RAS, the amino acid of the molecule stretch that corresponds to position 13 of the G13C RAS would be L-cysteine or D-cysteine or a cysteine analogue, preferably L-cysteine. In connection with G13C RAS, the aforementioned comments on the inclusion of an unprotected cysteine in the molecule apply. Hence, in certain embodiments, in a molecule directed to G13C mutant human RAS, the amino acid of the molecule stretch that corresponds to position 13 of the G13C RAS would be L-serine or D-serine or a serine analogue, preferably L-serine. In certain other embodiments, in a molecule directed to G13C mutant human RAS, the amino acid of the molecule stretch that corresponds to position 13 of the G13C RAS would be L-cysteine or D-cysteine or a cysteine analogue, preferably L-cysteine, having its —SH group protected by a protective group or participating in a disulphide bridge.

Accordingly, in certain embodiments the molecule as taught herein directed to a G12 mutant human RAS, particularly to a G12V mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGA [symbol] GVG (SEQ ID NO: 2) including the valine at position 11 of SEQ ID NO: 2 (shown in bold), optionally wherein: a′) the molecule stretch includes at most 3, preferably at most 2, more preferably at most 1, and most preferably no single amino acid substitutions, b′) at least one amino acid of the molecule stretch is a D-amino acid, and/or c′) at least one amino acid of the molecule stretch is replaced by an analogue of the respective amino acid (where reference is made here and below to the aforementioned features a′), b′) and/or c′), features a′) and b′) and not c′) may apply, or features a′) and c′) and not b′) may apply, or features b′) and c′) and not a′) may apply, or features a′), b′) and c′) may all apply). In certain embodiments the molecule as taught herein directed to a G12 mutant human RAS, particularly to a G12V mutant human RAS, may contain a molecule stretch comprising 6 to 10 contiguous amino acids of SEQ ID NO: 2 including the valine at position 11 of SEQ ID NO: 2, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12V mutant human RAS, may contain a molecule stretch comprising 6 to 10 contiguous amino acids of SEQ ID NO: 2 including the valine at position 11 of SEQ ID NO: 2. Preferably, the molecule stretch may be N-terminally delimited by the amino acid at position 1, 2, 3, 4, 5, or 6 of SEQ ID NO: 2, more preferably by the amino acid at position 5 or 6 of SEQ ID NO: 2; and/or C-terminally delimited by the amino acid at position 11, 12, 13 or 14 of SEQ ID NO: 2, more preferably by the amino acid at position 12, 13 or 14 of SEQ ID NO: 2, even more preferably by the amino acid at position 13 or 14 of SEQ ID NO: 2, still more preferably by the amino acid at position 14 of SEQ ID NO: 2.

In certain embodiments the molecule as taught herein directed to a G12 mutant human RAS, particularly to a G12V mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGA [symbol] GVG (SEQ ID NO: 26) including the valine at position 7 of SEQ ID NO: 26 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12V mutant human RAS, may contain a molecule stretch comprising 6 to 10 contiguous amino acids of SEQ ID NO: 26 including the valine at position 7 of SEQ ID NO: 26, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12V mutant human RAS, may contain a molecule stretch comprising 6 to 10 contiguous amino acids of SEQ ID NO: 26 including the valine at position 7 of SEQ ID NO: 26. Preferably, the molecule stretch may be N-terminally delimited by the amino acid at position 1, 2, or 3 of SEQ ID NO: 26, more preferably by the amino acid at position 1 or 2 of SEQ ID NO: 26; and/or C-terminally delimited by the amino acid at position 7, 8, 9 or 10 of SEQ ID NO: 26, more preferably by the amino acid at position 8, 9 or 10 of SEQ ID NO: 26, even more preferably by the amino acid at position 9 or 10 of SEQ ID NO: 26, still more preferably by the amino acid at position 10 of SEQ ID NO: 26.

Non-limiting examples of the contiguous portions of SEQ ID NO: 26 that may define the span and boundaries of the molecule stretch are shown in Table 1 below. The first row of the table reproduces SEQ ID NO: 26 and each subsequent row exemplifies a particular molecule stretch based on SEQ ID NO: 26 by indicating the amino acids of SEQ ID NO: 26 that are included (“+”) vs. not included (“−”) in the molecule stretch.

TABLE 1

| L | V | V | V | G | A | V | G | V | G |
|---|---|---|---|---|---|---|---|---|---|
| + | + | + | + | + | + | + | + | + | + |
| − | + | + | + | + | + | + | + | + | + |
| − | − | + | + | + | + | + | + | + | + |
| − | − | − | + | + | + | + | + | + | + |
| + | + | + | + | + | + | + | + | + | − |
| − | + | + | + | + | + | + | + | + | − |
| − | − | + | + | + | + | + | + | + | − |
| − | − | − | + | + | + | + | + | + | − |
| + | + | + | + | + | + | + | + | − | − |
| − | + | + | + | + | + | + | + | − | − |
| − | − | + | + | + | + | + | + | − | − |
| + | + | + | + | + | + | + | − | − | − |
| − | + | + | + | + | + | + | − | − | - |

In certain embodiments the molecule as taught herein directed to a G12 mutant human RAS, particularly to a G12C mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGA [symbol] GVG (SEQ ID NO: 6) or preferably TEYKLVVVGA [symbol] GV (SEQ ID NO: 3) including the cysteine at position 11 of SEQ ID NO: 6 or 3 (shown in bold) (as explained elsewhere in this specification, the cysteine may, in each of the embodiments described in this and the ensuing two paragraphs and Table 2 be swapped for a serine or protected by a suitable protective group or a disulphide bridge) (where here and below a number of contiguous amino acids is recited that exceeds the number of amino acids actually present in the recited sequence, it shall be understood that the maximum number of contiguous amino acids coincides with the number of amino acids constituting the sequence), optionally wherein: a′) the molecule stretch includes at most 3, preferably at most 2, more preferably at most 1, and most preferably no single amino acid substitutions, b′) at least one amino acid of the molecule stretch is a D-amino acid, and/or c′) at least one amino acid of the molecule stretch is replaced by an analogue of the respective amino acid. In certain embodiments the molecule as taught herein directed to a G12 mutant human RAS, particularly to a G12C mutant human RAS, may contain a molecule stretch comprising 6 to 10 contiguous amino acids of SEQ ID NO: 6 or 3 including the cysteine at position 11 of SEQ ID NO: 6 or 3, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12C mutant human RAS, may contain a molecule stretch comprising 6 to 10 contiguous amino acids of SEQ ID NO: 6 or 3 including the cysteine at position 11 of SEQ ID NO: 6 or 3. Preferably, the molecule stretch may be N-terminally delimited by the amino acid at position 1, 2, 3, 4, 5, or 6 of SEQ ID NO: 6, more preferably by the amino acid at position 5 or 6 of SEQ ID NO: 6; and/or C-terminally delimited by the amino acid at position 11, 12, 13 or 14 of SEQ ID NO: 6, more preferably by the amino acid at position 12, 13 or 14 of SEQ ID NO: 6, even more preferably by the amino acid at position 13 or 14 of SEQ ID NO: 6, still more preferably by the amino acid at position 13 of SEQ ID NO: 6.

In certain embodiments the molecule as taught herein directed to a G12 mutant human RAS, particularly to a G12C mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGA [symbol] GVG (SEQ ID NO: 27) or preferably LVVVGA [symbol] GV (SEQ ID NO: 28) including the cysteine at position 7 of SEQ ID NO: 27 or 28 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12C mutant human RAS, may contain a molecule stretch comprising 6 to 10 contiguous amino acids of SEQ ID NO: 27 or 28 including the cysteine at position 7 of SEQ ID NO: 27 or 28, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12C mutant human RAS, may contain a molecule stretch comprising 6 to 10 contiguous amino acids of SEQ ID NO: 27 or 28 including the cysteine at position 7 of SEQ ID NO: 27 or 28. Preferably, the molecule stretch may be N-terminally delimited by the amino acid at position 1, 2, or 3 of SEQ ID NO: 27, more preferably by the amino acid at position 1 or 2 of SEQ ID NO: 27; and/or C-terminally delimited by the amino acid at position 7, 8, 9 or 10 of SEQ ID NO: 27, more preferably by the amino acid at position 8, 9 or 10 of SEQ ID NO: 27, even more preferably by the amino acid at position 9 or 10 of SEQ ID NO: 27, still more preferably by the amino acid at position 9 of SEQ ID NO: 27.

Non-limiting examples of the contiguous portions of SEQ ID NO: 27 that may define the span and boundaries of the molecule stretch are shown in Table 2 below. The first row of the table reproduces SEQ ID NO: 27 and each subsequent row exemplifies a particular molecule stretch based on SEQ ID NO: 27 by indicating the amino acids of SEQ ID NO: 27 that are included (“+”) vs. not included (“−”) in the molecule stretch.

TABLE 2

| L | V | V | V | G | A | C | G | V | G |
|---|---|---|---|---|---|---|---|---|---|
| + | + | + | + | + | + | + | + | + | + |
| − | + | + | + | + | + | + | + | + | + |
| − | − | + | + | + | + | + | + | + | + |
| − | − | − | + | + | + | + | + | + | + |
| + | + | + | + | + | + | + | + | + | − |
| − | + | + | + | + | + | + | + | + | − |
| − | − | + | + | + | + | + | + | + | − |
| − | − | − | + | + | + | + | + | + | − |
| + | + | + | + | + | + | + | + | − | − |
| − | + | + | + | + | + | + | + | − | − |
| − | − | + | + | + | + | + | + | − | − |
| + | + | + | + | + | + | + | − | − | − |
| − | + | + | + | + | + | + | − | − | − |

In certain embodiments the molecule as taught herein directed to a G12 mutant human RAS, particularly to a G12A mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGA [symbol] GVG (SEQ ID NO: 7) or preferably TEYKLVVVGA [symbol] GV (SEQ ID NO: 4) including the alanine at position 11 of SEQ ID NO: 7 or 4 (shown in bold), optionally wherein: a′) the molecule stretch includes at most 3, preferably at most 2, more preferably at most 1, and most preferably no single amino acid substitutions, b′) at least one amino acid of the molecule stretch is a D-amino acid, and /or c′) at least one amino acid of the molecule stretch is replaced by an analogue of the respective amino acid. In certain embodiments the molecule as taught herein directed to a G12 mutant human RAS, particularly to a G12A mutant human RAS, may contain a molecule stretch comprising 6 to 10 contiguous amino acids of SEQ ID NO: 7 or 4 including the alanine at position 11 of SEQ ID NO: 7 or 4, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12A mutant human RAS, may contain a molecule stretch comprising 6 to 10 contiguous amino acids of SEQ ID NO: 7 or 4 including the alanine at position 11 of SEQ ID NO: 7 or 4. Preferably, the molecule stretch may be N-terminally delimited by the amino acid at position 1, 2, 3, 4, 5, or 6 of SEQ ID NO: 7, more preferably by the amino acid at position 5 or 6 of SEQ ID NO: 7; and/or C-terminally delimited by the amino acid at position 11, 12, 13 or 14 of SEQ ID NO: 7, more preferably by the amino acid at position 12, 13 or 14 of SEQ ID NO: 7, even more preferably by the amino acid at position 13 or 14 of SEQ ID NO: 7, still more preferably by the amino acid at position 13 of SEQ ID NO: 7.

In certain embodiments the molecule as taught herein directed to a G12 mutant human RAS, particularly to a G12A mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGA [symbol] GVG (SEQ ID NO: 29) or preferably LVVVGA [symbol] GV (SEQ ID NO: 30) including the alanine at position 7 of SEQ ID NO: 29 or 30 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12A mutant human RAS, may contain a molecule stretch comprising 6 to 10 contiguous amino acids of SEQ ID NO: 29 or 30 including the alanine at position 7 of SEQ ID NO: 29 or 30, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12A mutant human RAS, may contain a molecule stretch comprising 6 to 10 contiguous amino acids of SEQ ID NO: 29 or 30 including the alanine at position 7 of SEQ ID NO: 29 or 30. Preferably, the molecule stretch may be N-terminally delimited by the amino acid at position 1, 2, or 3 of SEQ ID NO: 29, more preferably by the amino acid at position 1 or 2 of SEQ ID NO: 29; and/or C-terminally delimited by the amino acid at position 7, 8, 9 or 10 of SEQ ID NO: 29, more preferably by the amino acid at position 8, 9 or 10 of SEQ ID NO: 29, even more preferably by the amino acid at position 9 or 10 of SEQ ID NO: 29, still more preferably by the amino acid at position 9 of SEQ ID NO: 29.

Non-limiting examples of the contiguous portions of SEQ ID NO: 29 that may define the span and boundaries of the molecule stretch are shown in Table 3 below. The first row of the table reproduces SEQ ID NO: 29 and each subsequent row exemplifies a particular molecule stretch based on SEQ ID NO: 29 by indicating the amino acids of SEQ ID NO: 29 that are included (“+”) vs. not included (“−”) in the molecule stretch.

TABLE 3

| L | V | V | V | G | A | A | G | V | G |
|---|---|---|---|---|---|---|---|---|---|
| + | + | + | + | + | + | + | + | + | + |
| − | + | + | + | + | + | + | + | + | + |
| − | − | + | + | + | + | + | + | + | + |
| − | − | − | + | + | + | + | + | + | + |
| + | + | + | + | + | + | + | + | + | − |
| − | + | + | + | + | + | + | + | + | − |
| − | − | + | + | + | + | + | + | + | − |
| − | − | − | + | + | + | + | + | + | − |
| + | + | + | + | + | + | + | + | − | − |
| − | + | + | + | + | + | + | + | − | − |
| − | − | + | + | + | + | + | + | − | − |
| + | + | + | + | + | + | + | − | − | − |
| − | + | + | + | + | + | + | − | − | − |

In certain embodiments the molecule as taught herein directed to a G12 mutant human RAS, particularly to a G12S mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGA [symbol] GVG (SEQ ID NO: 8) or preferably TEYKLVVVGA [symbol] GV (SEQ ID NO: 9) or more preferably TEYKLVVVGA [symbol] G (SEQ ID NO: 5) including the serine at position 11 of SEQ ID NO: 8, 9 or 5 (shown in bold), optionally wherein: a′) the molecule stretch includes at most 3, preferably at most 2, more preferably at most 1, and most preferably no single amino acid substitutions, b′) at least one amino acid of the molecule stretch is a D-amino acid, and/or c′) at least one amino acid of the molecule stretch is replaced by an analogue of the respective amino acid. In certain embodiments the molecule as taught herein directed to a G12 mutant human RAS, particularly to a G12S mutant human RAS, may contain a molecule stretch comprising 6 to 10 contiguous amino acids of SEQ ID NO: 8, 9 or 5 including the serine at position 11 of SEQ ID NO: 8, 9 or 5, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12S mutant human RAS, may contain a molecule stretch comprising 6 to 10 contiguous amino acids of SEQ ID NO: 8, 9 or 5 including the serine at position 11 of SEQ ID NO: 8, 9 or 5. Preferably, the molecule stretch may be N-terminally delimited by the amino acid at position 1, 2, 3, 4, 5, or 6 of SEQ ID NO: 8, more preferably by the amino acid at position 5 or 6 of SEQ ID NO: 8; and/or C-terminally delimited by the amino acid at position 11, 12, 13 or 14 of SEQ ID NO: 8, more preferably by the amino acid at position 12, 13 or 14 of SEQ ID NO: 8, even more preferably by the amino acid at position 12 or 13 of SEQ ID NO: 8, still more preferably by the amino acid at position 12 of SEQ ID NO: 8.

In certain embodiments the molecule as taught herein directed to a G12 mutant human RAS, particularly to a G12S mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGA [symbol] GVG (SEQ ID NO: 31) or preferably LVVVGA [symbol] GV (SEQ ID NO: 32) or more preferably LVVVGA [symbol] G (SEQ ID NO: 33) including the serine at position 7 of SEQ ID NO: 31, 32 or 33 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12S mutant human RAS, may contain a molecule stretch comprising 6 to 10 contiguous amino acids of SEQ ID NO: 31, 32 or 33 including the serine at position 7 of SEQ ID NO: 31, 32 or 33, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G12 mutant human RAS, particularly to a G12S mutant human RAS, may contain a molecule stretch comprising 6 to 10 contiguous amino acids of SEQ ID NO: 31, 32 or 33 including the serine at position 7 of SEQ ID NO: 31, 32 or 33. Preferably, the molecule stretch may be N-terminally delimited by the amino acid at position 1, 2, or 3 of SEQ ID NO: 31, more preferably by the amino acid at position 1 or 2 of SEQ ID NO: 31; and/or C-terminally delimited by the amino acid at position 7, 8, 9 or 10 of SEQ ID NO: 31, more preferably by the amino acid at position 8, 9 or 10 of SEQ ID NO: 31, even more preferably by the amino acid at position 8 or 9 of SEQ ID NO: 31, still more preferably by the amino acid at position 8 of SEQ ID NO: 31.

Non-limiting examples of the contiguous portions of SEQ ID NO: 31 that may define the span and boundaries of the molecule stretch are shown in Table 4 below. The first row of the table reproduces SEQ ID NO: 31 and each subsequent row exemplifies a particular molecule stretch based on SEQ ID NO: 31 by indicating the amino acids of SEQ ID NO: 29 that are included (“+”) vs. not included (“−”) in the molecule stretch.

TABLE 4

| L | V | V | V | G | A | S | G | V | G |
|---|---|---|---|---|---|---|---|---|---|
| + | + | + | + | + | + | + | + | + | + |
| − | + | + | + | + | + | + | + | + | + |
| − | − | + | + | + | + | + | + | + | + |
| − | − | − | + | + | + | + | + | + | + |
| + | + | + | + | + | + | + | + | + | − |
| − | + | + | + | + | + | + | + | + | − |
| − | − | + | + | + | + | + | + | + | − |
| − | − | − | + | + | + | + | + | + | − |
| + | + | + | + | + | + | + | + | − | − |
| − | + | + | + | + | + | + | + | − | − |
| − | − | + | + | + | + | + | + | − | − |
| + | + | + | + | + | + | + | − | − | − |
| − | + | + | + | + | + | + | − | − | − |

In certain particularly preferred embodiments, the molecule as taught herein directed to a G12 mutant human RAS, particularly to a G12V mutant human RAS, may contain the amino acid stretch VVVGAV (SEQ ID NO: 10), LVVVGAV (SEQ ID NO: 11), VVVGAVG (SEQ ID NO: 12) or VVVGAVGVG (SEQ ID NO: 13), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. Particularly preferably, the molecule may contain the amino acid stretch as shown in SEQ ID NO: 10, SEQ ID NO: 11, SEQ ID NO: 12 or SEQ ID NO: 13.

In certain embodiments the molecule as taught herein directed to a G13 mutant human RAS, particularly to a G13V mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGAG [symbol] VG (SEQ ID NO: 82) including the valine at position 12 of SEQ ID NO: 82 (shown in bold), optionally wherein: a′) the molecule stretch includes at most 3, preferably at most 2, more preferably at most 1, and most preferably no single amino acid substitutions, b′) at least one amino acid of the molecule stretch is a D-amino acid, and /or c′) at least one amino acid of the molecule stretch is replaced by an analogue of the respective amino acid (where reference is made here and below to the aforementioned features a′), b′) and/or c′), features a′) and b′) and not c′) may apply, or features a′) and c′) and not b′) may apply, or features b′) and c′) and not a′) may apply, or features a′), b′) and c′) may all apply). In certain embodiments the molecule as taught herein directed to a G13 mutant human RAS, particularly to a G13V mutant human RAS, may contain a molecule stretch comprising 6 to 10 contiguous amino acids of SEQ ID NO: 82 including the valine at position 12 of SEQ ID NO: 82, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13V mutant human RAS, may contain a molecule stretch comprising 6 to 10 contiguous amino acids of SEQ ID NO: 82 including the valine at position 12 of SEQ ID NO: 82. Preferably, the molecule stretch may be N-terminally delimited by the amino acid at position 1, 2, 3, 4, 5, or 6 of SEQ ID NO: 82, more preferably by the amino acid at position 5 or 6 of SEQ ID NO: 82; and/or C-terminally delimited by the amino acid at position 12, 13 or 14 of SEQ ID NO: 82, more preferably by the amino acid at position 13 or 14 of SEQ ID NO: 82, even more preferably by the amino acid at position 14 of SEQ ID NO: 82.

In certain embodiments the molecule as taught herein directed to a G13 mutant human RAS, particularly to a G13V mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGAG [symbol] VG (SEQ ID NO: 90) including the valine at position 8 of SEQ ID NO: 90 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13V mutant human RAS, may contain a molecule stretch comprising 6 to 10 contiguous amino acids of SEQ ID NO: 90 including the valine at position 8 of SEQ ID NO: 90, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13V mutant human RAS, may contain a molecule stretch comprising 6 to 10 contiguous amino acids of SEQ ID NO: 90 including the valine at position 8 of SEQ ID NO: 90. Preferably, the molecule stretch may be N-terminally delimited by the amino acid at position 1, 2, or 3 of SEQ ID NO: 90, more preferably by the amino acid at position 1 or 2 of SEQ ID NO: 90; and/or C-terminally delimited by the amino acid at position 8, 9 or 10 of SEQ ID NO: 90, more preferably by the amino acid at position 9 or 10 of SEQ ID NO: 90, even more preferably by the amino acid at position 10 of SEQ ID NO: 90.

In certain embodiments the molecule as taught herein directed to a G13 mutant human RAS, particularly to a G13C mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGAG [symbol] VG (SEQ ID NO: 91) or preferably TEYKLVVVGAG [symbol] V (SEQ ID NO: 81) including the cysteine at position 12 of SEQ ID NO: 91 or 81 (shown in bold) (as explained elsewhere in this specification, the cysteine may, in each of the embodiments described in this and the ensuing two paragraphs be swapped for a serine or protected by a suitable protective group or a disulphide bridge), optionally wherein: a′) the molecule stretch includes at most 3, preferably at most 2, more preferably at most 1, and most preferably no single amino acid substitutions, b′) at least one amino acid of the molecule stretch is a D-amino acid, and/or c′) at least one amino acid of the molecule stretch is replaced by an analogue of the respective amino acid. In certain embodiments the molecule as taught herein directed to a G13 mutant human RAS, particularly to a G13C mutant human RAS, may contain a molecule stretch comprising 6 to 10 contiguous amino acids of SEQ ID NO: 91 or 81 including the cysteine at position 12 of SEQ ID NO: 91 or 81, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13C mutant human RAS, may contain a molecule stretch comprising 6 to 10 contiguous amino acids of SEQ ID NO: 91 or 81 including the cysteine at position 12 of SEQ ID NO: 91 or 81. Preferably, the molecule stretch may be N-terminally delimited by the amino acid at position 1, 2, 3, 4, 5, or 6 of SEQ ID NO: 91, more preferably by the amino acid at position 5 or 6 of SEQ ID NO: 91; and/or C-terminally delimited by the amino acid at position 12, 13 or 14 of SEQ ID NO: 91, more preferably by the amino acid at position 13 of SEQ ID NO: 91.

In certain embodiments the molecule as taught herein directed to a G13 mutant human RAS, particularly to a G13C mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGAG [symbol] VG (SEQ ID NO: 92) or preferably LVVVGAG [symbol] V (SEQ ID NO: 93) including the cysteine at position 8 of SEQ ID NO: 92 or 93 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13C mutant human RAS, may contain a molecule stretch comprising 6 to 10 contiguous amino acids of SEQ ID NO: 92 or 93 including the cysteine at position 8 of SEQ ID NO: 92 or 93, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13C mutant human RAS, may contain a molecule stretch comprising 6 to 10 contiguous amino acids of SEQ ID NO: 92 or 93 including the cysteine at position 8 of SEQ ID NO: 92 or 93. Preferably, the molecule stretch may be N-terminally delimited by the amino acid at position 1, 2, or 3 of SEQ ID NO: 92, more preferably by the amino acid at position 1 or 2 of SEQ ID NO: 92; and/or C-terminally delimited by the amino acid at position 8, 9 or 10 of SEQ ID NO: 92, more preferably by the amino acid at position 9 of SEQ ID NO: 92.

In certain embodiments the molecule as taught herein directed to a G13 mutant human RAS, particularly to a G13S mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, such as exactly 10, or 11, 12, 13 or 14, more preferably 6 to 10, contiguous amino acids of the amino acid sequence TEYKLVVVGAG [symbol] VG (SEQ ID NO: 94) or preferably TEYKLVVVGAG [symbol] V (SEQ ID NO: 95) or more preferably TEYKLVVVGAG [symbol] (SEQ ID NO: 83) including the serine at position 12 of SEQ ID NO: 94, 95 or 83 (shown in bold), optionally wherein: a′) the molecule stretch includes at most 3, preferably at most 2, more preferably at most 1, and most preferably no single amino acid substitutions, b′) at least one amino acid of the molecule stretch is a D-amino acid, and /or c′) at least one amino acid of the molecule stretch is replaced by an analogue of the respective amino acid. In certain embodiments the molecule as taught herein directed to a G13 mutant human RAS, particularly to a G13S mutant human RAS, may contain a molecule stretch comprising 6 to 10 contiguous amino acids of SEQ ID NO: 94, 95 or 83 including the serine at position 12 of SEQ ID NO: 94, 95 or 83, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13S mutant human RAS, may contain a molecule stretch comprising 6 to 10 contiguous amino acids of SEQ ID NO: 94, 95 or 83 including the serine at position 12 of SEQ ID NO: 94, 95 or 83. Preferably, the molecule stretch may be N-terminally delimited by the amino acid at position 1, 2, 3, 4, 5, or 6 of SEQ ID NO: 94, more preferably by the amino acid at position 5 or 6 of SEQ ID NO: 94; and/or C-terminally delimited by the amino acid at position 12, 13 or 14 of SEQ ID NO: 94, more preferably by the amino acid at position 12 or 13 of SEQ ID NO: 94, even more preferably by the amino acid at position 12 of SEQ ID NO: 94.

In certain embodiments the molecule as taught herein directed to a G13 mutant human RAS, particularly to a G13S mutant human RAS, may contain a molecule stretch comprising at least 3, such as at least 4 or at least 5, preferably at least 6, such as exactly 6, or at least 7, such as exactly 7, or at least 8, such as exactly 8, or at least 9, such as exactly 9, or at least 10, contiguous amino acids of the amino acid sequence LVVVGAG [symbol] VG (SEQ ID NO: 96) or preferably LVVVGAG [symbol] V (SEQ ID NO: 97) or more preferably LVVVGAG [symbol] (SEQ ID NO: 98) including the serine at position 8 of SEQ ID NO: 96, 97 or 98 (shown in bold), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid.

In certain embodiments the molecule as taught herein directed to a G13 mutant human RAS, particularly to a G13S mutant human RAS, may contain a molecule stretch comprising 6 to 10 contiguous amino acids of SEQ ID NO: 96, 97 or 98 including the serine at position 8 of SEQ ID NO: 96, 97 or 98, optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. In certain embodiments the molecule as taught herein directed to a G13 mutant human RAS, particularly to a G13S mutant human RAS, may contain a molecule stretch comprising 6 to 10 contiguous amino acids of SEQ ID NO: 96, 97 or 98 including the serine at position 8 of SEQ ID NO: 96, 97 or 98. Preferably, the molecule stretch may be N-terminally delimited by the amino acid at position 1, 2, or 3 of SEQ ID NO: 96, more preferably by the amino acid at position 1 or 2 of SEQ ID NO: 96; and/or C-terminally delimited by the amino acid at position 8, 9 or 10 of SEQ ID NO: 96, more preferably by the amino acid at position 8 or 9 of SEQ ID NO: 96, even more preferably by the amino acid at position 8 of SEQ ID NO: 96.

In certain particularly preferred embodiments, the molecule as taught herein directed to a G13 mutant human RAS, particularly to a G13V mutant human RAS, may contain the amino acid stretch VVVGAGV (SEQ ID NO: 99), LVVVGAGV (SEQ ID NO: 100), VVVGAGVV (SEQ ID NO: 101), or VVVGAGVVG (SEQ ID NO: 102), optionally wherein: a′) the molecule stretch includes at most 3, preferably at most 2, more preferably at most 1, and most preferably no single amino acid substitutions; b′) at least one amino acid of the molecule stretch is a D-amino acid; and/or c′) at least one amino acid of the molecule stretch is replaced by an analogue of the respective amino acid. Particularly preferably, the molecule may contain the amino acid stretch as shown in SEQ ID NO: 99, SEQ ID NO: 100, SEQ ID NO: 101 or SEQ ID NO: 102.

At least certain molecules containing an amino acid stretch designed against a particular G12 mutation may also induce intermolecular beta-sheets and effect downregulation of mutant human RAS carrying a different G12 mutation. For example, as shown in the experimental section, molecules designed against G12V mutant human RAS, and containing a valine residue at the position corresponding to position 12 of RAS, could downregulate both G12V and G12C mutant human RAS, while substantially not impinging on wild-type human RAS. It is therefore envisaged herein that insofar the intended effects on downregulation of G12 mutant RAS are observed, molecules designed for one type of G12 mutation may be employed against mutant RAS carrying another type of G12 mutation. Also, at least certain molecules containing an amino acid stretch designed against a particular G13 mutation may also induce intermolecular beta-sheets and effect downregulation of mutant human RAS carrying a different G13 mutation. It is therefore envisaged herein that insofar the intended effects on downregulation of G13 mutant RAS are observed, molecules designed for one type of G13 mutation may be employed against mutant RAS carrying another type of G13 mutation.

Further, as illustrated above, the molecule stretch, i.e., the at least one amino acid stretch comprised by the molecules as taught herein which participates in the intermolecular beta-sheet, may also include D-amino acids and/or analogues of the recited amino acids. Stated more generally, in certain embodiments, the at least one amino acid stretch of the molecule may comprise one or more D-amino acids, or analogues of one or more of its amino acids, or one or more D-amino acids and analogues of one or more of its amino acids, provided the incorporation of the D-amino acid or D-amino acids and/or the analogue or analogues is compatible with the formation of the intermolecular beta-sheet as taught herein.

Without limitation, in certain embodiments the molecule stretch may include only one D-amino acid. In certain embodiments, the molecule stretch may include two or more (e.g., 3, 4, 5, 6 or more) D-amino acids. In certain embodiments, about 10%, about 20%, about 30%, about 40%, about 50%, about 60%, about 70%, about 80%, about 90%, or 100% (i.e., all) amino acids constituting the molecule stretch may be D-amino acids. In certain embodiments, the D-amino acids may be interspersed between L-amino acids and/or the D-amino acids may be organised into one or more sub-stretches of two or more D-amino acids separated by L-amino acids. Without limitation, in certain embodiments the molecule stretch may include an analogue of only one of its amino acids. In certain embodiments, the molecule stretch may include analogues of two or more (e.g., 3, 4, 5, 6 or more) of its amino acids. In certain embodiments, the molecule stretch may include analogues of about 10%, about 20%, about 30%, about 40%, about 50%, about 60%, about 70%, about 80%, about 90%, or 100% (i.e., all) of its amino acids. In certain embodiments, the amino acid analogues may be interspersed between naturally occurring amino acids and/or the amino acid analogues may be organised into one or more sub-stretches of two or more such analogues separated by naturally occurring amino acids. Without limitation, in certain embodiments the molecule stretch may include only one constituent that is a D-amino acid or a amino acid analogue. In certain embodiments, the molecule stretch may include two or more (e.g., 3, 4, 5, 6 or more) constituents that are D-amino acids or amino acid analogues. In certain embodiments, about 10%, about 20%, about 30%, about 40%, about 50%, about 60%, about 70%, about 80%, about 90%, or 100% (i.e., all) constituents of the molecule stretch may be D-amino acids or amino acid analogues.

As already explained, the molecule stretch may be designed to correspond to the RAS stretch, which may in particular call for a certain degree of sequence identity between the molecule stretch and the RAS stretch. For example, the molecule stretch may be most preferably identical to the RAS stretch, or may differ from the latter only by single amino acid substitution(s), in particular by no more than 3, preferably no more than 2, more preferably no more than 1 single amino acid substitutions. Such comparatively high extent of sequence identity between the molecule stretch and the RAS stretch aims to allow the stretches to associate, in particular through the formation of an intermolecular beta-sheet there between. It has indeed been reported that ‘self-association’ of beta-aggregating regions within naturally occurring proteins is a widespread underlying mechanism of aggregation of such proteins (see for example Fernandez-Escamilla et al. 2004, supra), and the present approach is able to take advantage of this. As also already explained, the notion of correspondence between a molecule stretch and a RAS stretch does allow for the inclusion of D-isomers and/or analogues of the respective amino acids in the molecule stretch.

The reference to an amino acid analogue may encompass any compound that has the same or similar basic chemical structure as a naturally-encoded amino acid, i.e., an organic compound comprising a carboxyl group, an amino group, and an R moiety (amino acid residue). Typically, the amino group and the R moiety may be bound to the α carbon atom (i.e., the carbon atom to which the carboxyl group is bound). In other embodiments, the amino group may be bound to a carbon atom other than the α carbon atom, for example, to the β or γ carbon atom, preferably to the β carbon atom. In such embodiments, the R moiety may be bound to the same carbon atom as the amino group or to a carbon atom closer to the α carbon atom or to the α carbon atom itself. Typically, where the carboxyl group, the amino group and the R moiety are bound to the α carbon atom, the α carbon atom may also be bound to a hydrogen atom. Typically, where the amino group and the R moiety are bound to the β carbon atom, the β carbon atom may also be bound to a hydrogen atom. Without limitation, the R moiety of an amino acid analogue may differ from the R group of the respective naturally-encoded amino acid by one or more individual atoms or functional groups of the R group being replaced or substituted with a different atom (e.g., a methyl group replaced with a hydrogen atom, or an S atom replaced with an O atom, etc.), with an isotope of the same atom (e.g., ¹²C replaced with ¹³C, ¹⁴N replaced with ¹⁵N, or ¹H replaced with ²H, etc.), or with a different functional group (e.g., a hydrogen atom replaced with a methyl, ethyl or propyl group, or with another alkyl, alkenyl, cycloalkyl, cycloalkenyl, heterocyclyl, aryl, or heteroaryl group; an —SH group replaced with an —OH group or —NH₂ group, etc.). The structural difference or modification in an amino acid analogue compared to the respective naturally-encoded amino acid preferably preserves the core property of the amino acid with respect to charge and polarity. Hence, an amino acid analogue of a non-polar hydrophobic amino acid may preferably also have a non-polar hydrophobic R moiety; an amino acid analogue of a polar neutral amino acid may preferably also have a polar neutral R moiety; an amino acid analogue of a positively charged (basic) amino acid may preferably also have a positively charged R moiety, preferably with the same number of charged groups; and an amino acid analogue of a negatively charged (acidic) amino acid may preferably also have negatively charged R moiety, preferably with the same number of charged groups. All amino acid analogues are envisaged as both D- and L-stereoisomers, provided their structure allows such stereoisomeric forms.

By means of an example and without limitation, a leucine analogue may be selected from the list consisting of 2-amino-3,3-dimethyl-butyric acid (t-Leucine), alpha-methylleucine, hydroxyleucine, 2,3-dehydro-leucine, N-alpha-methyl-leucine, 2-Amino-5-methyl-hexanoic acid (homoleucine), 3-Amino-5-methylhexanoic acid (beta-homoleucine), 2-Amino-4,4-dimethyl-pentanoic acid (4-methyl-leucine, neopentylglycine), 4,5-dehydro-norleucine, L-norleucine, N-alpha-methyl-norleucine, and 6-hydroxy-norleucine, including their D- and L-stereoisomers, provided their structure allows such stereoisomeric forms. By means of an example and without limitation, a valine analogue may be selected from the list consisting of c-alpha-methyl-valine (2,3-dimethylbutanoic acid), 2,3-dehydro-valine, 3,4-dehydro-valine, 3-methyl-L-isovaline (methylvaline), 2-amino-3-hydroxy-3-methylbutanoic acid (hydroxyvaline), beta-homovaline, and N-alpha-methyl-valine, including their D-and L-stereoisomers, provided their structure allows such stereoisomeric forms. By means of an example and without limitation, a glycine analogue may be selected from the list consisting of N-alpha-methyl-glycine (sarcosine), cyclopropylglycine, and cyclopentylglycine, including their D- and L-stereoisomers, provided their structure allows such stereoisomeric forms. By means of an example and without limitation, an alanine analogue may be selected from the list consisting of 2-amino-isobutyric acid (2-methylalanine), 2-amino-2-methylbutanoic acid (isovaline), N-alpha-methyl-alanine, c-alpha-methyl-alanine, c-alpha-ethyl-alanine, 2-amino-2-methylpent-4-enoic acid (alpha-allylalanine), beta-homoalanine, 2-indanyl-glycine, di-n-propyl-glycine, di-n-butyl-glycine, diethylglycine, (1-naphthyl)alanine, (2 -naphthyl)alanine, cyclohexylglycine, cyclopropylglycine, cyclopentylglycine, adamantyl-glycine, and beta-homoallylglycine, including their D- and L-stereoisomers, provided their structure allows such stereoisomeric forms.

In certain embodiments, the molecule may comprise exactly one amino acid stretch which participates in the intermolecular beta-sheet (i.e., exactly one ‘molecule stretch’ as discussed above). In certain preferred embodiments, the molecule may comprise two or more amino acid stretches which participate in the intermolecular beta-sheet (i.e., two or more ‘molecule stretches’ as discussed above). For example, the molecule may comprise 2 to 6, preferably 2 to 5, more preferably 2 to 4, or even more preferably 2 or 3 molecule stretches. For example, the molecule may comprise exactly 2, or exactly 3, or exactly 4, or exactly 5 molecule stretches, particularly preferably exactly 2 or exactly 3 molecule stretches, even more preferably exactly 2 molecule stretches. The inclusion of two or more molecule stretches tends to increase the effectiveness of the molecules in downregulating and inducing aggregation of the respective G12 mutant human RAS proteins. Hence, in preferred embodiments, the two or more molecule stretches will be directed to the same G12 or G13 mutant RAS. However, a configuration where the two or more molecule stretches are directed to different G12 mutant human RAS proteins can be envisaged, and can provide for a more universal G12 targeting agent. A configuration where the two or more molecule stretches are directed to different G13 mutant human RAS proteins can be envisaged, and can provide for a more universal G13 targeting agent.

Where the molecule comprises two or more molecule stretches as taught herein, these may each independently be identical or different. For example, in a molecule with exactly 2 molecule stretches, the 2 molecule stretches may be identical or different; in a molecule with exactly 3 molecule stretches, all 3 stretches may be identical, or each stretch may be different from each other stretch, or 2 stretches may be identical and the remaining stretch may be different; or in a molecule with exactly 4 molecule stretches, all 4 stretches may be identical, or each stretch may be different from each other stretch, or 2 or 3 stretches may be identical and the remaining stretch(es) may be different from the former and optionally identical to each other.

By means of examples and without limitation, where two molecule stretches are said to be different, each molecule stretch may correspond to a different RAS stretch as taught herein, such as for example to non-overlapping, overlapping, or nested, but nonetheless different, RAS stretches, preferably of the same G12 or G13 mutant RAS. In such embodiments, the two molecule stretches may be designed with different underlying amino acid sequences in mind, and may optionally also differ in other respects such as in the extent to which they incorporate (or not) amino acid substitutions, D-isomers and/or analogues of the respective amino acids. Or where two molecule stretches are said to be different, each molecule stretch may correspond to the same RAS stretch, such that the two molecule stretches are designed with the same underlying amino acid sequence in mind, but can differ in other respects such as in the extent to which they incorporate (or not) amino acid substitutions, D-isomers and/or analogues of the respective amino acids. In particularly preferred embodiments, the two or more molecule stretches correspond to the same RAS stretch, more preferably the two or more molecule stretches do not differ in amino acid substitutions (e.g., they might not incorporate any amino acid substitutions compared to the RAS stretch or may incorporate the same amino acid substitutions), and even more preferably also do not differ in the extent to which they incorporate D-isomers and/or analogues of the respective amino acids (e.g., they might not incorporate any D-isomers and/or analogues or may incorporate the same D-isomers and/or analogues at the same position(s)). Hence, in particularly preferred embodiments, the two or more molecule stretches are identical.

Where the molecule comprises two or more amino acid stretches which participate in the intermolecular beta-sheet (i.e., two or more ‘molecule stretches’ as discussed above), the reference to “the intermolecular beta-sheet” does not necessarily denote physically the same beta-sheet, but may denote another beta-sheet with another RAS protein molecule. For example, a molecule with two molecule stretches may engage two RAS protein molecules in the same beta-sheet, or in two separate beta-sheets, or initially in two separate beta-sheets which later become part of the same beta-sheet or the same higher order structure driven by beta-sheet formation. Hence, what is particularly sought is the occurrence of conformational changes in the N-terminal most APR of G12 or G13 mutant RAS molecules towards beta-strands and beta-sheets, which eventually decreases solubility and causes aggregation of the G12 or G13 mutant RAS.

In preferred embodiments, to reduce the propensity of the molecules containing the above-discussed amino acid stretch or stretches to self-associate or self-aggregate even before being exposed to their target RAS protein (e.g., to precipitate upon production or during storage), the amino acid stretch or stretches may be enclosed or gated by amino acids that can reduce or prevent such self-association (also termed “gatekeeper amino acids” or “gatekeepers”). Accordingly, in certain embodiments, the amino acid stretch or stretches within the molecule are each independently flanked, in particular directly or immediately flanked, on each end independently, by one or more amino acids, in particular contiguous amino acids, that display low beta-sheet forming potential or a propensity to disrupt beta-sheets. Typically, such flanking regions may each independently comprise 1 to 10, preferably 1 to 8, more preferably 1 to 6, or even more preferably 1 to 4, such as exactly 1, exactly 2, exactly 3 or exactly 4 amino acids, particularly contiguous amino acids, that have low beta-sheet forming potential or propensity to disrupt beta-sheets.

In certain preferred embodiments, an amino acid having low beta-sheet forming potential or propensity to disrupt beta-sheets may be a charged amino acid, such as a positively charged (basic, such as overall +1 or +2 charge) amino acid or a negatively charged (acidic, such as overall −1 or −2 charge) amino acid, such as an amino acid containing an amino group (—NH₃⁺ when protonated) or a carboxyl group (—COO⁻ when dissociated) in its R moiety. In certain other embodiments, an amino acid having low beta-sheet forming potential or propensity to disrupt beta-sheets may be an amino acid typified by high conformational rigidity, for example due to the inclusion of its peptide bond-forming amino group in a heterocycle, such as in pyrrolidine.

Hence, in certain preferred embodiments, an amino acid having low beta-sheet forming potential or propensity to disrupt beta-sheets may be R, K, E, D, P, N, S, H, G, Q, or A, including D- and L-stereoisomers thereof, or analogues thereof In certain preferred embodiments, an amino acid having low beta-sheet forming potential or propensity to disrupt beta-sheets may be R, K, E, D, P, N, S, H, G or Q, including D- and L-stereoisomers thereof, or analogues thereof. In certain more preferred embodiments, an amino acid having low beta-sheet forming potential or propensity to disrupt beta-sheets may be R, K, E, D or P, including D- and L-stereoisomers thereof, or analogues thereof. In certain more preferred embodiments, an amino acid having low beta-sheet forming potential or propensity to disrupt beta-sheets may be R, K, E or D, including D- and L-stereoisomers thereof, or analogues thereof. Accordingly, in certain embodiments, the amino acid stretch or stretches within the molecule are each independently flanked, on each end independently, by one or more amino acids, preferably by 1 to 4 contiguous amino acids, selected from the group consisting of R, K, E, D, P, N, S, H, G, Q, and A, D- and L-stereoisomers thereof, and analogues thereof, and combinations thereof; or selected from the group consisting of R, K, E, D, P, N, S, H, G, and Q, D- and L-stereoisomers thereof, and analogues thereof, and combinations thereof; or selected from the group consisting of R, K, E, D, and P, D- and L-stereoisomers thereof, and analogues thereof, and combinations thereof.

By means of an example and without limitation, an arginine analogue, in particular an arginine analogue that carries a positive charge or can be protonated to carry a positive charge, may be selected from the list consisting of 2-amino-3-ureido-propionic acid, norarginine, 2-amino-3-guanidino-propionic acid, glyoxal-hydroimidazolone, methylglyoxal-hydroimidazolone, N′-nitro-arginine, homoarginine, omega-methyl-arginine, N-alpha-methyl-arginine, N,N′-diethyl-homoarginine, canavanine, and beta-homoarginine, including their D- and L-stereoisomers, provided their structure allows such stereoisomeric forms. By means of an example and without limitation, a lysine analogue, in particular a lysine analogue that carries a positive charge or can be protonated to carry a positive charge, may be selected from the list consisting of N-epsilon-formyl-lysine, N-epsilon-methyl-lysine, N-epsilon-i-propyl-lysine, N-epsilon-dimethyl-lysine, N-epsilon-trimethylamonium-lysine, N-epsilon-nicotinyl-lysine, ornithine, N-delta-methyl-ornithine, N-delta-N-delta-dimethyl-ornithine, N-delta-i-propyl-ornithine, c-alpha-methyl-ornithine, beta,beta-dimethyl-ornithine, N-delta-methyl-N-delta-butyl-ornithine, N-delta-methyl-N-delta-phenyl-ornithine, c-alpha-methyl-lysine, beta,beta-dimethyl-lysine, N-alpha-methyl-lysine, homolysine, and beta-homolysine, including their D- and L-stereoisomers, provided their structure allows such stereoisomeric forms. By means of an example and without limitation, a glutamic or aspartic acid analogue, in particular a glutamic or aspartic acid analogue that carries a negative charge or can dissociate to carry a negative charge, may be selected from the list consisting of 2-amino-adipic acid (homoglutamic acid), 2-amino-heptanedioic acid (2-aminopimelic acid), 2-amino-octanedioic acid (aminosuberic acid), and 2-amino-4-carboxy-pentanedioic acid (4-carboxy glutamic acid), including their D- and L-stereoisomers, provided their structure allows such stereoisomeric forms. By means of an example and without limitation, a proline analogue may be selected from the list consisting of 3 -methylproline, 3,4-dehydro-proline, 2-[(2 S)-2 -(hydrazinecarbonyl)pyrrolidin- 1 -yl]-2-oxoacetic acid, beta-homoproline, alpha-methyl-proline, hydroxyproline, 4-oxo-proline, beta,beta-dimethyl-proline, 5,5-dimethyl-proline, 4-cyclohexyl-proline, 4-phenyl-proline, 3-phenyl-proline, and 4-aminoproline, including their D- and L-stereoisomers, provided their structure allows such stereoisomeric forms. A further non-limiting example of an amino acid that may be included in a gatekeeper moiety or moieties as disclosed herein, possibly in combination with other amino acids, is diaminopimelic acid. A further non-limiting example of an amino acid that may be included in a gatekeeper moiety or moieties as disclosed herein, possibly in combination with other amino acids, is citrulline.

By means an illustration and without limitation, examples of such gatekeeper sequences or regions that can flank the molecule stretches may be, each independently, R, K, E, D, P, A, diaminopimelic acid, citrulline, RR, KK, EE, DD, PP, RK, KR, ED, DE, RRR, KKK, DDD, EEE, PPP, RRK, RKK, KKR, KRR, RKR, KRK, DDE, DEE, EED, EDD, EDE, or DED, etc., wherein any arginine, lysine, glutamate, aspartate, proline, or alanine may be L- or D-isomer, and optionally wherein any arginine, lysine, glutamate, aspartate, proline, or alanine may be substituted by its analogue as discussed elsewhere in this specification.

As discussed earlier, the molecules can comprise at least one portion that can assume or mimic a beta-strand conformation capable of interacting with the beta-strand contributed by the RAS protein APR so as to give rise to an intermolecular beta-sheet formed by said interacting beta-strands, while in certain embodiments, such portion may preferably be an amino acid stretch (molecule stretch') which participates in the intermolecular beta-sheet. In certain other embodiments, the portion may be a peptidomimetic of such a molecule stretch. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The chemical nature and structure of the molecules outside of the portions that are intended to interlock with the beta-strands of the RAS APR, such as in other words outside of the ‘molecule stretch or stretches’ as discussed hitherto, is comparatively less critical, insofar these remaining sections or portions of the molecule do not interfere with or preferably facilitate or enable the aforementioned intermolecular beta-sheet interaction.

In certain embodiments, where the molecule comprises two or more RAS-interacting molecule stretches as discussed herein, each optionally and preferably flanked by gatekeeper regions, these molecule stretches are connected, in particular covalently connected, directly or preferably through a linker (also known as spacer). The incorporation of such linkers or spacers may endow the individual molecule stretches with more conformational freedom and less steric hindrance to interact with RAS. Optionally, in addition to being interposed between the molecule stretches, linkers may also be added outside of the first and/or outside of the last molecule stretch of the molecule. This applies mutatis mutandis for molecules only including one molecule stretch, optionally and preferably flanked by gatekeeper regions, wherein linkers may be coupled to one or both ends of the single molecule stretch.

The nature and structure of such linkers is not particularly limited. The linker may be a rigid linker or a flexible linker. In particular embodiments, the linker is a covalent linker, achieving a covalent bond. The terms “covalent” or “covalent bond” refer to a chemical bond that involves the sharing of one or more electron pairs between two atoms. A linker may be, for example, a (poly)peptide or non-peptide linker, such as a non-peptide polymer, such as a non-biological polymer. Preferably, any linkages may be hydrolytically stable linkages, i.e., substantially stable in water at useful pH values, including in particular under physiological conditions, for an extended period of time, e.g., for days.

In certain embodiments, each linker may be independently selected from a stretch of between 1 and 20 identical or non-identical units, wherein a unit is an amino acid, a monosaccharide, a nucleotide or a monomer. Non-identical units can be non-identical units of the same nature (e.g. different amino acids, or some copolymers). They can also be non-identical units of a different nature, e.g. a linker with amino acid and nucleotide units, or a heteropolymer (copolymer) comprising two or more different monomeric species. According to specific embodiments, each linker may be independently composed of 1 to 10 units of the same nature, particularly of 1 to 5 units of the same nature. According to particular embodiments, all linkers present in the molecule may be of the same nature, or may be identical.

In particular embodiments, any one linker may be a peptide or polypeptide linker of one or more amino acids. In certain embodiments, all linkers in the molecule may be peptide or polypeptide linkers. More particularly, the peptide linker may be 1 to 20 amino acids long, such as preferably 1 to 10 amino acids long, such as more preferably 2 to 5 amino acids long. For example, the linker may be exactly 1, 2, 3, 4, 5, 6, 7, 8, 9, or 10 amino acids long, such as preferably exactly 2, 3 or 4 amino acids long. The nature of amino acids constituting the linker is not of particular relevance so long as the biological activity of the molecule stretches linked thereby is not substantially impaired. Preferred linkers are essentially non-immunogenic and/or not prone to proteolytic cleavage. In certain embodiments, the linker may contain a predicted secondary structure such as an alpha-helical structure. However, linkers predicted to assume flexible, random coil structures are preferred. Linkers having tendency to form beta-strands may be less preferred or may need to be avoided. Cysteine residues may be less preferred or may need to be avoided due to their capacity to form intermolecular disulphide bridges. Basic or acidic amino acid residues, such as arginine, lysine, histidine, aspartic acid and glutamic acid may be less preferred or may need to be avoided due to their capacity for unintended electrostatic interactions. In certain preferred embodiments, the peptide linker may comprise, consist essentially of or consist of amino acids selected from the group consisting of glycine, serine, alanine, phenylalanine, threonine, proline, and combinations thereof, including D-isomers and analogues thereof. In certain preferred embodiments, the peptide linker may comprise, consist essentially of or consist of amino acids selected from the group consisting of glycine, serine, alanine, threonine, proline, and combinations thereof, including D-isomers and analogues thereof. In even more preferred embodiments, the peptide linker may comprise, consist essentially of or consist of amino acids selected from the group consisting of glycine, serine, and combinations thereof, including D-isomers and analogues thereof. In certain embodiments, the peptide linker may consist of only glycine and serine residues. In certain embodiments, the peptide linker may consist of only glycine residues or analogues thereof, preferably of only glycine residues. In certain embodiments, the peptide linker may consist of only serine residues or D-isomers or analogues thereof, preferably of only serine residues. Such linkers provide for particularly good flexibility. In certain embodiments, the linker may consist essentially of or consist of glycine and serine residues. In certain embodiments, the glycine and serine residues may be present at a ratio between 4:1 and 1:4 (by number), such as about 3:1, about 2:1, about 1:1, about 1:2 or about 1:3 glycine : serine. Preferably, glycine may be more abundant than serine, e.g., a ratio between 4:1 and 1.5:1 glycine : serine, such as about 3:1 or about 2:1 glycine : serine (by number). In certain embodiments, the N-terminal and C-terminal residues of the linker are both a serine residue; or the N-terminal and C-terminal residues of the linker are both glycine residues; or the N-terminal residue is a serine residue and the C-terminal residue is a glycine residue; or the N-terminal residue is a glycine residue and the C-terminal residue is a serine residue. In certain embodiments, the peptide linker may consist of only proline residues or D-isomers or analogues thereof, preferably of only proline residues. By means of examples and without limitation, peptide linkers as intended herein may be e.g. PP, PPP, GS, SG, SGG, SSG, GSS, GGS, GSGS (SEQ ID NO: 14), AS, SA, GF, FF, etc.

In certain embodiments, the linker may be a non-peptide linker. In preferred embodiments, the non-peptide linker may comprise, consist essentially of or consist of a non-peptide polymer. The term “non-peptide polymer” as used herein refers to a biocompatible polymer including two or more repeating units linked to each other by a covalent bond excluding the peptide bond. For example, the non-peptide polymer may be 2 to 200 units long or 2 to 100 units long or 2 to 50 units long or 2 to 45 units long or 2 to 40 units long or 2 to 35 units long or 2 to 30 units long or 5 to 25 units long or 5 to 20 units long or 5 to 15 units long. The non-peptide polymer may be selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (polylactic-glycolic acid), lipid polymers, chitins, hyaluronic acid, and combinations thereof. Particularly preferred is poly(ethylene glycol) (PEG). Another particularly envisaged chemical linker is Ttds (4,7,10-trioxatridecan-13-succinamic acid). The molecular weight of the non-peptide polymer preferably may range from 1 to 100 kDa, and preferably 1 to 20 kDa. The non-peptide polymer may be one polymer or a combination of different types of polymers. The non-peptide polymer has reactive groups capable of binding to the elements which are to be coupled by the linker. Preferably, the non-peptide polymer has a reactive group at each end. Preferably, the reactive group is selected from the group consisting of a reactive aldehyde group, a propione aldehyde group, a butyl aldehyde group, a maleimide group and a succinimide derivative. The succinimide derivative may be succinimidyl propionate, hydroxy succinimidyl, succinimidyl carboxymethyl or succinimidyl carbonate. The reactive groups at both ends of the non-peptide polymer may be the same or different. In certain embodiments, the non-peptide polymer has a reactive aldehyde group at both ends. For example, the non-peptide polymer may possess a maleimide group at one end and, at the other end, an aldehyde group, a propionic aldehyde group or a butyl aldehyde group. When a polyethylene glycol (PEG) having a reactive hydroxy group at both ends thereof is used as the non-peptide polymer, the hydroxy group may be activated to various reactive groups by known chemical reactions, or a PEG having a commercially-available modified reactive group may be used so as to prepare the protein conjugate.

In certain particularly preferred embodiments, the operative part of the molecule, i.e., the part responsible for the effects on RAS, may be a peptide. Put differently, in such embodiments, the molecule stretch or stretches that form beta-strands interacting with the RAS APR, the optional and preferred flanking gatekeeper regions, the linkers optionally and preferably interposed between the molecule stretches, and the linkers optionally but less preferably added outside of the outermost molecule stretches, are all composed of amino acids (which may include D- and L-stereoisomers and amino acid analogues) covalently linked by peptide bonds. Preferably, the total length of such peptide operative part of the molecule does not exceed 50 amino acids, such as does not exceed 45, 40, 35, 30, 25 or even 20 amino acids. Such peptide operative part of the molecule may be coupled to one or more other moieties, which themselves may but need not be amino acids, peptides, or polypeptides, and which may serve other functions, such as allowing to detect the molecule, increasing the half-life of the molecule when administered to subjects, increasing the solubility of the molecule, increasing the cellular uptake of the molecule, etc., as discussed elsewhere in this specification. In certain particularly preferred embodiments, the molecule is a peptide. Preferably, the total length of such peptide does not exceed 50 amino acids, such as does not exceed 45, 40, 35, 30, 25 or even 20 amino acids. Where the molecule comprises, consists essentially of or consists of, e.g., is, a peptide the N-terminus of said molecule can be modified, such as for example by acetylation, and/or the C-terminus of said molecule can be modified, such as for example by amidation.

In view of the foregoing discussion, in certain embodiments, the molecule as taught herein may be conveniently represented as comprising, consisting essentially of or consisting of the structure:

a) NGK1-P1-CGK1,

b) NGK1 -P1-CGK1-Z 1-NGK2-P2-CGK2,

c) NGK1 -P1-CGK1-Z 1-NGK2-P2-CGK2-Z2-NGK3-P3 -CGK3, or

d) NGK1 -P1-CGK1-Z 1-NGK2-P2-CGK2-Z2-NGK3 -P3-CGK3-Z3-NGK4-P4-CGK4,

wherein:

P1 to P4 each independently denote the amino acid stretch (molecule stretch') as taught above, NGK1 to NGK4 and CGK1 to CGK4 each independently denote the gatekeeper region as taught above, and

Z1 to Z3 each independently denote a direct bond or preferably the linker as taught above.

Hence, structure a) refers to a molecule only containing one molecule stretch as taught herein, while structures b), c) and d) refer to molecules containing two, three or four molecule stretch as taught herein, respectively.

In certain embodiments, as explained above, NGK1 to NGK4 and CGK1 to CGK4 may each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof. In certain embodiments, NGK1 to NGK4 and CGK1 to CGK4 may each independently denote 1 to 2 contiguous amino acids selected from the group consisting of R, K, A, and D, D-isomers and/or analogues thereof, and combinations thereof, such as NGK1 to NGK4 and CGK1 to CGK4 may be each independently K, R, D, A, or KK. In certain particularly preferred embodiments, NGK1 to NGK4 and CGK1 to CGK4 may each independently denote 1 to 2 contiguous amino acids selected from the group consisting of R, K, and D, D-isomers and/or analogues thereof, and combinations thereof, such as NGK1 to NGK4 and CGK1 to CGK4 may be each independently K, R, D or KK.

In certain particularly preferred embodiments, each linker is independently selected from a stretch of between 1 and 10 units, preferably between 1 and 5 units, wherein a unit is each independently an amino acid or PEG, such as each linker is independently GS, PP, AS, SA, GF, FF, or GSGS (SEQ ID NO: 14), or D-isomers and/or analogues thereof, preferably each linker is independently GS, PP or GSGS (SEQ ID NO: 14), preferably GS, or D-isomers and/or analogues thereof. In certain preferred embodiments, each independently, a direct bond is included instead of a linker.

In certain preferred embodiments, the molecule comprises, consists essentially of or consists of a peptide of the structure:

a) Gate-Pept-Gate;

b) Linker-Gate-Pept-Gate;

c) Gate-Pept-Gate-Linker;

d) Linker-Gate-Pept-Gate-Linker;

e) Gate-Pept-Gate-(Linker)-Gate-Pept-Gate;

f) Linker-Gate-Pept-Gate-(Linker)-Gate-Pept-Gate;

g) Gate-Pept-Gate-(Linker)-Gate-Pept-Gate-Linker;

h) Linker-Gate-Pept-Gate-(Linker)-Gate-Pept-Gate-Linker;

i) Gate-Pept-Gate-(Linker)-Gate-Pept-Gate-(Linker)-Gate-Pept-Gate;

j) Linker-Gate-Pept-Gate-(Linker)-Gate-Pept-Gate-(Linker)-Gate-Pept-Gate;

k) Gate-Pept-Gate-(Linker)-Gate-Pept-Gate-(Linker)-Gate-Pept-Gate-Linker; or

l) Linker-Gate-Pept-Gate-(Linker)-Gate-Pept-Gate-(Linker)-Gate-Pept-Gate-Linker;

wherein “Gate”, “Pept”, and “Linker” denote peptide elements bound to the adjacent peptide element(s) by peptide bond(s), wherein left-to-right order of the peptide elements signifies their N- to C-terminal organisation in the peptide;

wherein “Pept” (directed against G12 mutant RAS) is each independently LVVVGAVGVG (SEQ ID NO: 26), VVVGAVGVG (SEQ ID NO: 13), VVGAVGVG (SEQ ID NO: 34), VGAVGVG (SEQ ID NO: 35), LVVVGAVGV (SEQ ID NO: 36), VVVGAVGV (SEQ ID NO: 37), VVGAVGV (SEQ ID NO: 38), VGAVGV (SEQ ID NO: 39), LVVVGAVG (SEQ ID NO: 40), VVVGAVG (SEQ ID NO: 12), VVGAVG (SEQ ID NO: 41), LVVVGAV (SEQ ID NO: 11), VVVGAV (SEQ ID NO: 10), LVVVGACGVG (SEQ ID NO: 27), VVVGACGVG (SEQ ID NO: 42), VVGACGVG (SEQ ID NO: 43), VGACGVG (SEQ ID NO: 44), LVVVGACGV (SEQ ID NO: 28), VVVGACGV (SEQ ID NO: 45), VVGACGV (SEQ ID NO: 46), VGACGV (SEQ ID NO: 47), LVVVGACG (SEQ ID NO: 48), VVVGACG (SEQ ID NO: 49), VVGACG (SEQ ID NO: 30), LVVVGAC (SEQ ID NO: 51), VVVGAC (SEQ ID NO: 52), LVVVGAAGVG (SEQ ID NO: 29), VVVGAAGVG (SEQ ID NO: 53), VVGAAGVG (SEQ ID NO: 54), VGAAGVG (SEQ ID NO: 55), LVVVGAAGV (SEQ ID NO: 30), VVVGAAGV (SEQ ID NO: 56), VVGAAGV (SEQ ID NO: 57), VGAAGV (SEQ ID NO: 58), LVVVGAAG (SEQ ID NO: 59), VVVGAAG (SEQ ID NO: 60), VVGAAG (SEQ ID NO: 61), LVVVGAA (SEQ ID NO: 62), VVVGAA (SEQ ID NO: 63), LVVVGASGVG (SEQ ID NO: 31), VVVGASGVG (SEQ ID NO: 64), VVGASGVG (SEQ ID NO: 65), VGASGVG (SEQ ID NO: 66), LVVVGASGV (SEQ ID NO: 32), VVVGASGV (SEQ ID NO: 67), VVGASGV (SEQ ID NO: 68), VGASGV (SEQ ID NO: 69), LVVVGASG (SEQ ID NO: 33), VVVGASG (SEQ ID NO: 70), VVGASG (SEQ ID NO: 71), LVVVGAS (SEQ ID NO: 72), or VVVGAS (SEQ ID NO: 73), optionally wherein any one or more or all of the recited amino acids is or are replaced by its or their D-isomer(s) or by its or their analogue(s), including L- and D-isomers of such analogue(s) (as explained elsewhere in this specification, the cysteine may, in any “Pept” denoted as containing cysteine, be swapped for a serine or protected by a suitable protective group or a disulphide bridge), or wherein “Pept” (directed against G13 mutant RAS) is each independently LVVVGAGVVG (SEQ ID NO: 90), VVVGAGVVG (SEQ ID NO: 102), VVGAGVVG (SEQ ID NO: 103), VGAGVVG (SEQ ID NO: 104), GAGVVG (SEQ ID NO: 105), LVVVGAGVV (SEQ ID NO: 106), VVVGAGVV (SEQ ID NO: 107), VVGAGVV (SEQ ID NO: 108), VGAGVV (SEQ ID NO: 109), LVVVGAGV (SEQ ID NO: 100), VVVGAGV (SEQ ID NO: 99), VVGAGV (SEQ ID NO: 110), LVVVGAGCVG (SEQ ID NO: 92), VVVGAGCVG (SEQ ID NO: 109), VVGAGCVG (SEQ ID NO: 110), VGAGCVG (SEQ ID NO: 111), GAGCVG (SEQ ID NO: 112), LVVVGAGCV (SEQ ID NO: 93), VVVGAGCV (SEQ ID NO: 113), VVGAGCV (SEQ ID NO: 114), VGAGCV (SEQ ID NO: 115), LVVVGAGC (SEQ ID NO: 116), VVVGAGC (SEQ ID NO: 117), VVGAGC (SEQ ID NO: 118), LVVVGAGSVG (SEQ ID NO: 96), VVVGAGSVG (SEQ ID NO: 119), VVGAGSVG (SEQ ID NO: 120), VGAGSVG (SEQ ID NO: 121), GAGSVG (SEQ ID NO: 122), LVVVGAGSV (SEQ ID NO: 97), VVVGAGSV (SEQ ID NO: 123), VVGAGSV (SEQ ID NO: 124), VGAGSV (SEQ ID NO: 125), LVVVGAGS (SEQ ID NO: 98), VVVGAGS (SEQ ID NO: 126), VVGAGS (SEQ ID NO: 127), optionally wherein any one or more or all of the recited amino acids is or are replaced by its or their D-isomer(s) or by its or their analogue(s), including L- and D-isomers of such analogue(s) (as explained elsewhere in this specification, the cysteine may, in any “Pept” denoted as containing cysteine, be swapped for a serine or protected by a suitable protective group or a disulphide bridge);

wherein “Gate” is each independently lysine (K) or D-lysine or D- or L-lysine analogue (preferably lysine), arginine (R) or D-arginine or D- or L-arginine analogue (preferably arginine), aspartic acid (D) or D-aspartic acid or D- or L-aspartic acid analogue (preferably aspartic acid), glutamic acid (E) or D-glutamic acid or D- or L-glutamic acid analogue (preferably glutamic acid), KK, KKK, KKKK (SEQ ID NO: 50), RR, RRR, RRRR (SEQ ID NO: 133), DD, DDD, DDDD (SEQ ID NO: 134), EE, EEE, EEEE (SEQ ID NO: 135), KR, RK, KKR, KRK, RKK, RRK, RKR, KRR, KRKR (SEQ ID NO: 136), KRRK (SEQ ID NO: 137), RKKR (SEQ ID NO: 138), DE, ED, DDE, DED, EED, EED, EDE, DEE, DEDE (SEQ ID NO: 139), DEED (SEQ ID NO: 140), or EDDE (SEQ ID NO: 141), optionally wherein any one or more or all of the recited amino acids is or are replaced by its or their D-isomer(s) or by its or their analogue(s), including L- and D-isomers of such analogue(s); and

wherein the inclusion of the word “Linker” in parentheses denotes that the linker, each independently, may be absent or is preferably present, and wherein “Linker” is each independently glycine (G) or D- or L-glycine analogue (preferably glycine), serine (S) or D-serine or D- or L-serine analogue (preferably serine), proline (P) or D-proline or D- or L-proline analogue (preferably proline), GG, GGG, GGGG (SEQ ID NO: 142), SS, SSS, SSSS (SEQ ID NO: 143), GS, SG, GGS, GSG, SGG, SSG, SGS, SSG, GGGS (SEQ ID NO: 144), GGSG (SEQ ID NO: 145), GSGG (SEQ ID NO: 146), SGGG (SEQ ID NO: 147), GGSS (SEQ ID NO: 148), GSSG (SEQ ID NO: 149), SSGG (SEQ ID NO: 150), GSGS (SEQ ID NO: 14), SGSG (SEQ ID NO: 151), GSGSG (SEQ ID NO: 152), SGSGS (SEQ ID NO: 153), PP, PPP, or PPPP (SEQ ID NO: 154), optionally wherein any one or more or all of the recited amino acids is or are replaced by its or their D-isomer(s) or by its or their analogue(s), including L- and D-isomers of such analogue(s).

In such peptides, the N-terminal amino acid may be modified such as acetylated and/or the C-terminal amino acid may be modified such as amidated. In such peptides, D-amino acid(s) and or amino acid analogue(s) can be incorporated as long as their incorporation is compatible with the formation of the intermolecular beta-sheet as taught herein.

In certain particularly preferred embodiments, the molecule comprises, consists essentially of or consists of any one of the peptides individualised in Table 5, wherein each row of Table 5 denotes an individual peptide, wherein each cell within a given row of Table 5 specifies an individual peptide element included in the peptide individualised by that row and said peptide element is bound to the peptide element(s) adjacent to it by peptide bond(s), wherein the left to right order of the cells in a given row of Table 5 signifies the N- to C-terminal organisation of the peptide elements specified by those cells in the peptide individualised by that row, wherein:

for “Pept”, “i” is VVVGAV (SEQ ID NO: 10) or VVVGAGV (SEQ ID NO: 99), preferably SEQ ID NO: 10, “ii” is LVVVGAV (SEQ ID NO: 11) LVVVGAGV (SEQ ID NO: 100), preferably SEQ ID NO: 11, “iii” is VVVGAVG (SEQ ID NO: 12) or VVVGAGVV (SEQ ID NO: 101), preferably SEQ ID NO: 12, “iv” is VVVGAVGVG (SEQ ID NO: 13) or VVVGAGVVG (SEQ ID NO: 102), preferably SEQ ID NO: 13, optionally wherein any one or more or all of the recited amino acids is or are replaced by its or their D-isomer(s) or by its or their analogue(s), including L- and D-isomers of such analogue(s); preferably for “Pept”, “i” is VVVGAV (SEQ ID NO: 10), “ii” is LVVVGAV (SEQ ID NO: 11), “iii” is VVVGAVG (SEQ ID NO: 12), “iv” is VVVGAVGVG (SEQ ID NO: 13);

for “Gate”, “i” is lysine (K) or D-lysine or D- or L-lysine analogue, preferably lysine, “ii” is arginine (R) or D-arginine or D- or L-arginine analogue, preferably arginine, “iii” is aspartic acid (D) or D-aspartic acid or D- or L-aspartic acid analogue, preferably aspartic acid, “iv” is KK, optionally wherein any one or more or all of the recited amino acids is or are replaced by its or their D-isomer(s) or by its or their analogue(s), including L- and D-isomers of such analogue(s); preferably for “Gate”, “i” is K, “ii” R, “iii” is D, “iv” is KK; and

for “Linker”, which may be absent or is preferably present, “i” is GS, “ii” is GSG, “iii” is GSGS (SEQ ID NO: 14), “iv” is PP, optionally wherein any one or more or all of the recited amino acids is or are replaced by its or their D-isomer(s) or by its or their analogue(s), including L- and D-isomers of such analogue(s); preferably for “Linker”, “i” is GS, “ii” is GSG, “iii” is GSGS (SEQ ID NO: 14), “iv” is PP.

TABLE 5

| Gate | Pept | Gate | Linker | Gate | Pept | Gate |
|---|---|---|---|---|---|---|
| i | i | i | i | i | i | i |
| ii | i | ii | i | ii | i | ii |
| iii | i | iii | i | iii | i | iii |
| iv | i | iv | i | iv | i | iv |
| iv | i | i | i | i | i | iv |
| i | i | i | ii | i | i | i |
| ii | i | ii | ii | ii | i | ii |
| iii | i | iii | ii | iii | i | iii |
| iv | i | iv | ii | iv | i | iv |
| iv | i | i | ii | i | i | iv |
| i | i | i | iii | i | i | i |
| ii | i | ii | iii | ii | i | ii |
| iii | i | iii | iii | iii | i | iii |
| iv | i | iv | iii | iv | i | iv |
| iv | i | i | iii | i | i | iv |
| i | i | i | iv | i | i | i |
| ii | i | ii | iv | ii | i | ii |
| iii | i | iii | iv | iii | i | iii |
| iv | i | iv | iv | iv | i | iv |
| iv | i | i | iv | i | i | iv |
| i | ii | i | i | i | ii | i |
| ii | ii | ii | i | ii | ii | ii |
| iii | ii | iii | i | iii | ii | iii |
| iv | ii | iv | i | iv | ii | iv |
| iv | ii | i | i | i | ii | iv |
| i | ii | i | ii | i | ii | i |
| ii | ii | ii | ii | ii | ii | ii |
| iii | ii | iii | ii | iii | ii | iii |
| iv | ii | iv | ii | iv | ii | iv |
| iv | ii | i | ii | i | ii | iv |
| i | ii | i | iii | i | ii | i |
| ii | ii | ii | iii | ii | ii | ii |
| iii | ii | iii | iii | iii | ii | iii |
| iv | ii | iv | iii | iv | ii | iv |
| iv | ii | i | iii | i | ii | iv |
| i | ii | i | iv | i | ii | i |
| ii | ii | ii | iv | ii | ii | ii |
| iii | ii | iii | iv | iii | ii | iii |
| iv | ii | iv | iv | iv | ii | iv |
| iv | ii | i | iv | i | ii | iv |
| i | iii | i | i | i | iii | i |
| ii | iii | ii | i | ii | iii | ii |
| iii | iii | iii | i | iii | iii | iii |
| iv | iii | iv | i | iv | iii | iv |
| iv | iii | i | i | i | iii | iv |
| i | iii | i | ii | i | iii | i |
| ii | iii | ii | ii | ii | iii | ii |
| iii | iii | iii | ii | iii | iii | iii |
| iv | iii | iv | ii | iv | iii | iv |
| iv | iii | i | ii | i | iii | iv |
| i | iii | i | iii | i | iii | i |
| ii | iii | ii | iii | ii | iii | ii |
| iii | iii | iii | iii | iii | iii | iii |
| iv | iii | iv | iii | iv | iii | iv |
| iv | iii | i | iii | i | iii | iv |
| i | iii | i | iv | i | iii | i |
| ii | iii | ii | iv | ii | iii | ii |
| iii | iii | iii | iv | iii | iii | iii |
| iv | iii | iv | iv | iv | iii | iv |
| iv | iii | i | iv | i | iii | iv |
| i | iv | i | i | i | iv | i |
| ii | iv | ii | i | ii | iv | ii |
| iii | iv | iii | i | iii | iv | iii |
| iv | iv | iv | i | iv | iv | iv |
| iv | iv | i | i | i | iv | iv |
| i | iv | i | ii | i | iv | i |
| ii | iv | ii | ii | ii | iv | ii |
| iii | iv | iii | ii | iii | iv | iii |
| iv | iv | iv | ii | iv | iv | iv |
| iv | iv | i | ii | i | iv | iv |
| i | iv | i | iii | i | iv | i |
| ii | iv | ii | iii | ii | iv | ii |
| iii | iv | iii | iii | iii | iv | iii |
| iv | iv | iv | iii | iv | iv | iv |
| iv | iv | i | iii | i | iv | iv |
| i | iv | i | iv | i | iv | i |
| ii | iv | ii | iv | ii | iv | ii |
| iii | iv | iii | iv | iii | iv | iii |
| iv | iv | iv | iv | iv | iv | iv |
| iv | iv | i | iv | i | iv | iv |

In peptides as individualised in Table 5, the N-terminal amino acid may be modified such as acetylated and/or the C-terminal amino acid may be modified such as amidated. In such peptides, D-amino acid(s) and or amino acid analogue(s) can be incorporated as long as their incorporation is compatible with the formation of the intermolecular beta-sheet as taught herein.

Any peptide as individualised in Table 5 may have an additional linker, as discussed elsewhere in this specification or more particularly as defined in connection with Table 5, added or fused to the N-terminus, or to the C-terminus, or to both N- and C-termini each independently.

In certain particularly preferred embodiments, the molecule comprises, consists essentially of or consists of a peptide of the amino acid sequence:

a)
(SEQ ID NO: 15)
KVVVGAVKGSKVVVGAVK;
or
b)
(SEQ ID NO: 16)
KLVVVGAVKGSKLVVVGAVK;
or
c)
(SEQ ID NO: 17)
KVVVGAVGKGSKVVVGAVGK;
or
d)
(SEQ ID NO: 18)
KVVVGAVGVGKGSKVVVGAVGVGK;

optionally wherein the amino acid sequence comprises one or more D-amino acids and/or analogues of one or more of its amino acids, optionally wherein the N-terminal amino acid is acetylated and/or the C-terminal amino acid is amidated.

In certain particularly preferred embodiments, the molecule comprises, consists essentially of or consists of a peptide of the amino acid sequence: a) KVVVGAVKGSKVVVGAVK (SEQ ID NO: 15); b) KLVVVGAVKGSKLVVVGAVK (SEQ ID NO: 16); c) KVVVGAVGKGSKVVVGAVGK (SEQ ID NO: 17); or d) KVVVGAVGVGKGSKVVVGAVGVGK (SEQ ID NO: 18), optionally wherein the N-terminal amino acid is acetylated and/or the C-terminal amino acid is amidated.

In certain particularly preferred embodiments, the molecule consists of a peptide of the amino acid sequence: a) KVVVGAVKGSKVVVGAVK (SEQ ID NO: 15); b) KLVVVGAVKGSKLVVVGAVK (SEQ ID NO: 16); c) KVVVGAVGKGSKVVVGAVGK (SEQ ID NO: 17); or d) KVVVGAVGVGKGSKVVVGAVGVGK (SEQ ID NO: 18) optionally wherein the N-terminal amino acid is acetylated and/or the C-terminal amino acid is amidated.

In certain particularly preferred embodiments, the molecule comprises, consists essentially of or consists of a peptide of the amino acid sequence:

a)
(SEQ ID NO: 128)
KVVVGAGVKGSKVVVGAGVK;
or
b)
(SEQ ID NO: 129)
KLVVVGAGVKGSKLVVVGAGVK;
or
c)
(SEQ ID NO: 130)
KVVVGAGVVKGSKVVVGAGVVK;
or
d)
(SEQ ID NO: 131)
KVVVGAGVVGKGSKVVVGAGVVGK;

optionally wherein the amino acid sequence comprises one or more D-amino acids and/or analogues of one or more of its amino acids, optionally wherein the N-terminal amino acid is acetylated and/or the C-terminal amino acid is amidated.

In certain particularly preferred embodiments, the molecule comprises, consists essentially of or consists of a peptide of the amino acid sequence as shown in Table 12, such as SEQ ID NO: 157, 158-159, 161-176, 178, or 180-181, optionally wherein the amino acid sequence comprises one or more D-amino acids and/or analogues of one or more of its amino acids, optionally wherein the N-terminal amino acid is acetylated and/or the C-terminal amino acid is amidated. Hence, in certain particularly preferred embodiments, the molecule comprises, consists essentially of or consists of a peptide of the amino acid sequence:

a)
(SEQ ID NO: 160)
[Dap]LSVFAIKGSKLSVFAI[Dap];
or
b)
(SEQ ID NO: 161)
[Dap]VVVGAVKGSKVVVGAV[Dap];
or
c)
(SEQ ID NO: 162)
[Dap]VVVGAVGKGSKVVVGAVG[Dap];
or
d)
(SEQ ID NO: 163)
[Dap]VVVGAVGVGKGSKVVVGAVGVG[Dap];
or
e)
(SEQ ID NO: 164)
[Cit]VVVGAVKGSKVVVGAVK;
or
i)
(SEQ ID NO: 165)
KVVVGAV[Cit]GSKVVVGAVK;
or
g)
(SEQ ID NO: 166)
AVVVGAVKGSKVVVGAVK;
or
h)
(SEQ ID NO: 167)
KVVVGAVAGSKVVVGAVK;
or
i)
(SEQ ID NO: 168)
KVVVGAVKGSAVVVGAVK;
or
j)
(SEQ ID NO: 169)
KVVVGAVKGSKVVVGAVA;
or
k)
(SEQ ID NO: 170)
AVVVGAVKGSAVVVGAVK;
or
1)
(SEQ ID NO: 171)
KVVVGAVAGSKVVVGAVA;
or
m)
(SEQ ID NO: 172)
AVVVGAVAGSKVVVGAVK;
or
n)
(SEQ ID NO: 173)
KVVVGAVKASKVVVGAVK;
or
o)
(SEQ ID NO: 174)
KVVVGAVKGAKVVVGAVK;
or
p)
(SEQ ID NO: 175)
KVVVGAVGKGFKVVVGAVGK;
or
q)
(SEQ ID NO: 176)
KVVVGAVGKFFKVVVGAVGK;
or
r)
(SEQ ID NO: 178)
KVVVGAVGVGKKVVVGAVGVGK;

optionally wherein the amino acid sequence comprises one or more D-amino acids and/or analogues of one or more of its amino acids, optionally wherein the N-terminal amino acid is acetylated and/or the C-terminal amino acid is amidated (‘[Dap]’ denotes diaminopimelic acid, ‘[Cit]’ denotes citrulline).

In certain embodiments, the molecule as taught herein is not a peptide consisting of the amino acid sequence KLVVVGAVGV (SEQ ID NO: 182). In certain embodiments, the molecule as taught herein is not a peptide consisting of the amino acid sequence KLVVVGAVGVGKSALTI (SEQ ID NO: 183). In certain embodiments, the molecule as taught herein is not a peptide consisting of the amino acid sequence KLVVVGAVGVGKS (SEQ ID NO: 184).

As already touched upon above, in certain embodiments, the molecule as taught herein may comprise one or more further moieties, groups, components or parts, which may serve other functions or perform other roles and activities. Such functions, roles or activities may be useful or desired for example in connection with the production, synthesis, isolation, purification or formulation of the molecule, or in connection with its in experimental or therapeutic uses. Conveniently, the operative part of the molecule, i.e., the part responsible for the effects on RAS, may be connected to one or more such further moieties, groups, components or parts, preferably covalently connected, bound, linked or fused, directly or through a linker. Where such further moiety, group, component or part is a peptide, polypeptide or protein, the connection to the operative part of the molecule may preferably involve a peptide bond, direct one or through a peptide linker.

For all such added moieties, the nature of the fusion or linker is not vital to the invention, as long as the moiety and the molecule can exert their specific function. According to particular embodiments, the moieties which are fused to the molecules can be cleaved off, e.g. by using a linker moiety that has a protease recognition site. This way, the function of the moiety and the molecule can be separated, which may be particularly interesting for larger moieties, or for embodiments where the moiety is no longer necessary after a specific point in time, e.g., a tag that is cleaved off after a separation step using the tag.

In certain preferred embodiments, the molecule may comprise a detectable label, a moiety that allows for isolation of the molecule, a moiety increasing the stability of the molecule, a moiety increasing the solubility of the molecule, a moiety increasing the cellular uptake of the molecule, a moiety effecting targeting of the molecule to cells, or a combination of any two or more thereof. It shall be appreciated that a single moiety can carry out two or more functions or activities.

Hence, in certain embodiments the molecule may comprise a detectable label. The term “label” refers to any atom, molecule, moiety or biomolecule that may be used to provide a detectable and preferably quantifiable read-out or property, and that may be attached to or made part of an entity of interest, such as molecules as taught herein, such as peptides as taught herein. Labels may be suitably detectable by for example mass spectrometric, spectroscopic, optical, colourimetric, magnetic, photochemical, biochemical, immunochemical or chemical means. Labels include without limitation dyes; radiolabels such as isotopes of hydrogen, carbon, nitrogen, oxygen, phosphorous, sulphur, fluorine, chlorine, or iodine, such as ²H, ³H, ¹³C, ¹¹C, ¹⁴C, ¹⁵N, ¹⁸O, ¹⁷O, ³¹P, ³²P, ³³P, ³⁵S, ¹⁸F, ³⁶Cl, ¹²⁵I, or ¹³¹I respectively, electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.) alone or in combination with moieties that may suppress or shift emission spectra by fluorescence resonance energy transfer (FRET); and fluorescent proteins (e.g., GFP, RFP). Certain isotopically labelled molecules such as peptides as taught herein, for example those into which radioactive isotopes such as ³H and ¹⁴C are incorporated, are useful in drug and/or substrate tissue distribution assays. ³H and ¹⁴C isotopes are particularly preferred for their ease of preparation and detectability. Further, substitution with heavier isotopes such as ²H may afford certain therapeutic advantages resulting from greater metabolic stability, for example increased in vivo half-life or reduced dosage requirements and, hence, may be preferred in some circumstances. Isotopically labelled molecules such as peptides may generally be prepared by carrying production or synthesis methods in which a readily available isotopically labelled reagent is substituted for a non-isotopically labelled reagent. In some embodiments, the molecule may be provided with a tag that permits detection with another agent (e.g., with a probe binding partner). Such tags may be, for example, biotin, streptavidin, his-tag, myc tag, FLAG tag (DYKDDDDK, SEQ ID NO: 74), maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc. Labelled RAS-targeting molecules can lend themselves to a variety of uses and applications, such as without limitation, uses in in vitro assays, including diagnostic assays, where the labelled RAS-targeting pept-ins may provide a principle which binds to and allows for detection of RAS proteins of interest, such as mutant RAS proteins, in a biological sample from a subject; or use in in vivo imaging, where distribution of the labelled RAS-targeting pept-ins in the body may be followed by non-invasive imaging methods after administrations.

In further embodiments, the molecule may comprise a moiety that allows for the isolation (separation, purification) of the molecule. Typically, such moieties operate in conjunction with affinity purification methods, in which the ability to isolate a particular component of interest from other components is conferred by specific binding between a separable binding agent, such as an immunological binding agent (antibody), and the component of interest. Such affinity purification methods include without limitation affinity chromatography and magnetic particle separation. Such moieties are well-known in the art and non-limiting examples include biotin (isolatable using an affinity purification method utilising streptavidin), his-tag (isolatable using an affinity purification method utilising metal ion, e.g., Ni²⁺), maltose (isolatable using an affinity purification method utilising maltose binding protein), glutathione S-transferase (GST) (isolatable using an affinity purification method utilising glutathione), or myc or FLAG tag (isolatable using an affinity purification method utilising anti-myc or anti-FLAG antibody, respectively).

In further embodiments, the molecule may comprise a moiety that increases the solubility of the molecule. While the solubility of the molecules can be ensured and controlled by the inclusion of gatekeeper portions flanking the molecule stretch or stretches as discussed above, whereby this may in principle be sufficient to prevent premature aggregation of the molecules and keep them in solution, the further addition of a moiety that increases solubility, i.e., prevents aggregation, may provide easier handling of the molecules, and particularly improve their stability and shelf-life. Many of the labels and isolation tags discussed above will also increase the solubility of the molecule. Further, a well-known example of such solubilising moiety is PEG (polyethylene glycol). This moiety is particularly envisaged, as it can be used as linker as well as solubilising moiety. Other examples include peptides and proteins or protein domains, or even whole proteins, e.g. GFP. In this regard, it should be noted that, like PEG, one moiety can have different functions or effects. For instance, a FLAG tag is a peptide moiety that can be used as a label, but due to its charge density, it will also enhance solubilisation. PEGylation has already often been demonstrated to increase solubility of biopharmaceuticals (e.g., Veronese and Mero, BioDrugs. 2008; 22(5):315-29). Adding a peptide, polypeptide, protein or protein domain tag to a molecule of interest has been extensively described in the art. Examples include, but are not limited to, peptides derived from synuclein (e.g., Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G B1 domain, Protein A IgG ZZ repeat domain, and bacterial immunoglobulin binding domains (Hutt et al., J Biol Chem.; 287(7):4462-9, 2012). The nature of the tag will depend on the application, as can be determined by the skilled person. For instance, for transgenic expression of the molecules described herein, it might be envisaged to fuse the molecules to a larger domain to prevent premature degradation by the cellular machinery. Other applications may envisage fusion to a smaller solubilisation tag (e.g., less than 30 amino acids, or less than 20 amino acids, or even less than 10 amino acids) in order not to alter the properties of the molecules too much.

In further embodiments, the molecule may comprise a moiety increasing the stability of the molecule, e.g., the shelf-life of the molecule, and/or the half-life of the molecule, which may involve increasing the stability of the molecule and/or reducing the clearance of the molecule when administered. Such moieties may modulate pharmacokinetic and pharmacodynamic properties of the molecule. Many of the labels, isolation tags and solubilisation tags discussed above will also increase the shelf-life or in vivo half-life of the molecules, and the inclusion of D-amino acids and/or amino acid analogues may do so as well. For instance, it is known that fusion with albumin (e.g., human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. Strohl (BioDrugs. 2015, vol. 29, 215-39) reviews fusion protein-based strategies for half-life extension of biologics, including without limitation fusion to human IgG Fc domain, fusion to HSA, fusion to human transferrin, fusion to artificial gelatin-like protein (GLP), etc. In particular embodiments, the molecules are not fused to an agarose bead, a latex bead, a cellulose bead, a magnetic bead, a silica bead, a polyacrylamide bead, a microsphere, a glass bead or any solid support (e.g. polystyrene, plastic, nitrocellulose membrane, glass), or the NusA protein. However, these fusions are possible, and in specific embodiments, they are also envisaged.

In further embodiments, the molecule may comprise a moiety that increases the cellular uptake of the molecule. For example, the molecules can further comprise a sequence which mediates cell penetration (or cell translocation), i.e., the molecules are further modified through the recombinant or synthetic attachment of a cell penetration sequence. Cell-penetrating peptides (CPP) or protein transduction domain (PTD) sequences are well known in the art. The terms generally refer to peptides capable of entering into cells. This ability can be exploited for the delivery of molecules as disclosed herein to cells. Exemplary but non-limiting CPP include HIV-1 Tat-derived CPP (see, e.g., Frankel et al. 1988 (Science 240: 70-73)); Antennapedia peptides or penetratins (see, e.g., Derossi et al. 1994 (J Biol Chem 269: 10444-10450)); peptides derived from HSV-1 VP22 (see, e.g., Aints et al. 2001 (Gene Ther 8: 1051-1056)); transportans (see, e.g., Pooga et al. 1998 (FASEB J 12: 67-77)); protegrin 1 (PG-1) anti-microbial peptide SynB (Kokryakov et al. 1993 (FEBS Lett 327: 231-236)); model amphipathic (MAP) peptides (see, e.g., Oehlke et al. 1998 (Biochim Biophys Acta 1414: 127-139)); signal sequence-based cell-penetrating peptides (NLS) (see, e.g., Lin et al. 1995 (J Biol Chem 270: 14255-14258)); hydrophobic membrane translocating sequence (MTS) peptides (see, e.g., Lin et al. 1995, supra); and polyarginine, oligoarginine and arginine-rich peptides (see, e.g., Futaki et al. 2001 (J Biol Chem 276: 5836-5840)). Still other commonly used cell-permeable peptides (both natural and artificial peptides) are disclosed e.g. in Sawant and Torchilin, Mol Biosyst. 6(4):628-40, 2010; Noguchi et al., Cell Transplant. 19(6):649-54, 2010 and Lindgren and Langel, Methods Mol Biol. 683:3-19, 2011. The carrier peptides that have been derived from these proteins show little sequence homology with each other, but are all highly cationic and arginine or lysine rich. CPP can be of any length. For example CPP may be less than or equal to 500, 250, 150, 100, 50, 25, 10 or 6 amino acids in length. For example CPP may be greater than or equal to 4, 5, 6, 10, 25, 50, 100, 150 or 250 amino acids in length. Preferably, a CPP may be between 4 and 25 amino acids in length. The suitable length and design of the CPP will be easily determined by those skilled in the art. As a general reference on CPPs can serve inter alia “Cell penetrating peptides: processes and applications” (ed. Ulo Langel, 1st ed., CRC Press 2002); Advanced Drug Delivery Reviews 57: 489-660 (2005); Dietz & Bahr 2004 (Moll Cell Neurosci 27: 85-131)). An agent as disclosed herein may be conjugated with a CPP directly or indirectly, e.g., by means of a suitable linker, such as without limitation a PEG-based linker. Molecules described herein might not need a CPP to enter a cell. Indeed, as is shown in the examples, it is possible to target intracellular proteins, which require that the molecules are taken up by the cell, and this happens without fusion to a CPP.

In further embodiments, the molecule may comprise a moiety effecting targeting of the molecule to cells. For instance, the molecule may be fused to, e.g., an antibody, a peptide or a small molecule with a specificity for a given target, in particular with specificity to a cell expressing G12 or G13 mutant human RAS to which the molecule is directed, with specificity to a protein specifically expressed on the surface of that cell. In such embodiments, the molecule initiates downregulation or aggregation of the G12 or G13 mutant human RAS specifically in the targeted cells. In certain cases a binding domain is a chemical compound (e.g. a small compound with an affinity for at least one target protein) and in certain other cases a binding domain is a polypeptide, in certain other cases a binding domain is a protein domain. A protein binding domain is an element of overall protein structure that is self-stabilizing and often folds independently of the rest of the protein chain Binding domains vary in length from between about 25 amino acids up to 500 amino acids and more. Many binding domains can be classified into folds and are recognizable, identifiable, 3-D structures. Some folds are so common in many different proteins that they are given special names. Non-limiting examples are Rossman folds, TIM barrels, armadillo repeats, leucine zippers, cadherin domains, death effector domains, immunoglobulin-like domains, phosphotyrosine-binding domain, pleckstrin homology domain, src homology 2 domain, the BRCT domain of BRCA1 , G-protein binding domains, the Eps 15 homology (EH) domain and the protein-binding domain of p53. Antibodies are the natural prototype of specifically binding proteins with specificity mediated through hypervariable loop regions, so called complementary determining regions (CDR).

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a heavy-chain antibody, preferably a heavy-chain antibody derived from camelids; (2) by expression of a nucleotide sequence encoding a V_{HH} domain; (3) by “humanization” of a naturally occurring V_{HH} domain or by expression of a nucleic acid encoding a such humanized V_{HH} domain; (4) by “camelization” of a V_{H} domain from any animal species, and in particular from a mammalian species, such as from a human being, or by expression of a nucleic acid encoding such a camelized V_{H} domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

Although in general, antibody-like scaffolds have proven to work well as specific binders, it has become apparent that it is not compulsory to stick strictly to the paradigm of a rigid scaffold that displays CDR-like loops. In addition to antibodies, many other natural proteins mediate specific high-affinity interactions between domains. Alternatives to immunoglobulins have provided attractive starting points for the design of novel binding (recognition) molecules. The term scaffold, as used herein, refers to a protein framework that can carry altered amino acids or sequence insertions that confer binding to specific target proteins. Engineering scaffolds and designing libraries are mutually interdependent processes. In order to obtain specific binders, a combinatorial library of the scaffold has to be generated. This is usually done at the DNA level by randomizing the codons at appropriate amino acid positions, by using either degenerate codons or trinucleotides. A wide range of different non- immunoglobulin scaffolds with widely diverse origins and characteristics are currently used for combinatorial library display. Some of them are comparable in size to a scFv of an antibody (about 30 kDa), while the majority of them are much smaller. Modular scaffolds based on repeat proteins vary in size depending on the number of repetitive units. A non-limiting list of examples comprise binders based on the human 10th fibronectin type III domain, binders based on lipocalins, binders based on SH3 domains, binders based on members of the knottin family, binders based on CTLA-4, T-cell receptors, neocarzinostatin, carbohydrate binding module 4-2, tendamistat, kunitz domain inhibitors, PDZ domains, Src homology domain (SH2), scorpion toxins, insect defensin A, plant homeodomain finger proteins, bacterial enzyme TEM- 1 beta-lactamase, Ig-binding domain of Staphylococcus aureus protein A, E. coli colicin E7 immunity protein, E. coli cytochrome b562, ankyrin repeat domains. Hence, the term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat). Such scaffolds have been extensively reviewed in Binz et al., Gebauer and Skerra, Gill and Damle, Skerra 2000, and Skerra 2007, and include without limitation affibodies, based on the Z-domain of staphylococcal protein A, a three-helix bundle of 58 residues providing an interface on two of its alpha-helices (Nygren); engineered Kunitz domains based on a small (ca. 58 residues) and robust, disulphide-crosslinked serine protease inhibitor, typically of human origin (e.g. LACI-D1), which can be engineered for different protease specificities (Nixon and Wood); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra 2008); DARPins, designed ankyrin repeat domains (166 residues), which provide a rigid interface arising from typically three repeated beta-turns (Stumpp et al.); avimers (multimerized LDLR-A module) (Silverman et al.); and cysteine-rich knottin peptides (Kolmar). Also included as binding domains are compounds with a specificity for a given target protein, cyclic and linear peptide binders, peptide aptamers, multivalent avimer proteins or small modular immunopharmaceutical drugs, ligands with a specificity for a receptor or a co-receptor, protein binding partners identified in a two-hybrid analysis, binding domains based on the specificity of the biotin-avidin high affinity interaction, binding domains based on the specificity of cyclophilin-FK506 binding proteins. Also included are lectins with an affinity for a specific carbohydrate structure.

By means of an example, as G12 or G13 RAS mutations are often found in cancers, monoclonal antibodies fused to the present molecules may be configured to specifically bind a protein expressed by tumor cells in a subject, such as a tumor antigen, preferably a surface tumor antigen. The term “tumor antigen” refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc. The term further includes cancer/testis (CT) antigens. Examples of tumor antigens include, without limitation, β-human chorionic gonadotropin (βHCG), glycoprotein 100 (gp100/Pme117), carcinoembryonic antigen (CEA), tyrosinase, tyrosinase-related protein 1 (gp75/TRP1), tyrosinase-related protein 2 (TRP-2), NY-BR-1, NY-CO-58, NY-ESO-1, MN/gp250, idiotypes, telomerase, synovial sarcoma X breakpoint 2 (SSX2), mucin 1 (MUC-1), antigens of the melanoma-associated antigen (MAGE) family, high molecular weight-melanoma associated antigen (HMW-MAA), melanoma antigen recognized by T cells 1 (MART1), Wilms' tumor gene 1 (WT1), HER2/neu, mesothelin (MSLN), alphafetoprotein (AFP), cancer antigen 125 (CA-125), and abnormal forms of ras or p53. Further targets in neoplastic diseases include without limitation CD37 (chronic lymphocytic leukemia), CD123 (acute myeloid leukemia), CD30 (Hodgkin/large cell lymphoma), MET (NSCLC, gastroesophageal cancer), IL-6 (NSCLC), and GITR (malignant melanoma).

In those instances where other moieties are fused to the molecules, it is envisaged in particular embodiments that these moieties can be removed from the molecule. Typically, this will be done through incorporating a specific protease cleavage site or an equivalent approach. This is particularly the case where the moiety is a large protein: in such cases, the moiety may be cleaved off prior to using the molecule in any of the methods described herein (e.g. during purification of the molecules).

Note however that targeting moieties are not necessary, as the molecules themselves are able to find their target through specific sequence recognition. This may also allow, in alternative embodiments, to employ the molecules can as targeting moiety and be further fused to other moieties such as drugs, toxins or small molecules. By targeting the molecules to mutated RAS, these compounds can be targeted to the specific cell type/compartment. Thus, for instance, toxins can selectively be delivered to cancer cells expressing mutant RAS.

As the present invention makes use of the ‘interferon’ technology as generally described in WO 2007/071789A1 and WO2012/123419A1, and adopts this technology to the specific situation of G12 or G13 mutant human RAS where unexpected specificity of targeting is presently demonstrated, it shall be appreciated that the teachings of WO 2007/071789A1 and WO2012/123419A1 concerning the manners in which such ‘interferon’ molecules can be produced, isolated, purified, stored and formulated can be applied in the context of the present invention and need not be elaborated in great detail herein.

As mentioned, in particular embodiments, the operative part of the molecule may comprise, consist essentially of or consist of a peptide, preferably the operative part of the molecule may be a peptide. Moreover, in many embodiments, for example, where the operative part of the molecule is not connected or fused to other auxiliary moieties or where such additional moiety or moieties are themselves peptides, the entire molecule may be a peptide. Accordingly, standards tools and methods of chemical peptide synthesis, or of recombinant peptide or polypeptide production can be applied to the preparation of the present molecules. Recombinant protein production can also be applied to preparing molecules in which additional moiety or moieties which are themselves proteinaceous are included in the molecules and fused to the operative part of the molecule by peptide bonds.

Given that such techniques have become generally routine, in the interest of brevity, recombinant production of the present molecules may employ an expression cassette or expression vector comprising a nucleic acid encoding the molecule as taught herein and a promoter operably linked to the nucleic acid, wherein the expression cassette or expression vector is configured to effect expression of the molecule in a suitable host cell, such as a bacterial cell, a fungal cell, including yeast cells, an animal cell, or a mammalian cell, including human cells and non-human mammalian cells. Vectors may include plasmids, phagemids, bacteriophages, bacteriophage-derived vectors, PAC, BAC, linear nucleic acids, e.g., linear DNA, or viral vectors, etc. Expression vectors can be autonomous or integrative. Expression vectors can contain selection marker(s), e.g., URA3, TRP1, to permit detection and/or selection of the transformed cells. An operable linkage is a linkage in which regulatory sequences and sequences sought to be expressed are connected in such a way as to permit said expression. The promotor may be a constitutive or inducible (conditional) promoter, e.g., a chemically regulated or physically regulated inducible promoter. Non-limiting examples of promoters include T7, U6, H1, retroviral Rous sarcoma virus (RSV) LTR promoter, the cytomegalovirus (CMV) promoter, the metallothionein promoter, the adenovirus late promoter, the SV40 promoter, the dihydrofolate reductase promoter, the β-actin promoter, the phosphoglycerol kinase (PGK) promoter, and the EF 1 α promoter. Transcription terminators and optionally transcription enhancers may be included. A recombinant nucleic acid can be introduced into a host cell using a variety of methods such as direct injection, protoplasts fusion, calcium chloride, rubidium chloride, lithium chloride, calcium phosphate, DEAE dextran, cationic lipids or liposomes, biolistic particle bombardment (“gene gun” method), infection with viral vectors (e.g., derived from lentivirus, adeno-associated virus (AAV), adenovirus, retrovirus or antiviruses), electroporation, etc. Expression systems (host cells) that can be used for small or large scale production of peptides ro polypeptides include, without limitation, microorganisms such as bacteria (e.g., Escherichia. coli, Yersinia enterocolitica, Brucella sp., Salmonella tymphimurium, Serratia marcescens, or Bacillus subtilis), fungal cells (e.g., Yarrowia lipolytica, Arxula adeninivorans, methylotrophic yeast (e.g., methylotrophic yeast of the genus Candida, Hansenula, Oogataea, Pichia or Torulopsis, e.g., Pichia pastoris, Hansenula polymorpha, Ogataea minuta, or Pichia methanolica), or filamentous fungi of the genus Aspergillus, Trichoderma, Neurospora, Fusarium, or Chrysosporium, e.g., Aspergillus niger, Trichoderma reesei, or yeast of the genus Saccharomyces or Schizosaccharomyces, e.g., Saccharomyces cerevisiae, or Schizosaccharomyces pombe), insect cell systems (e.g., cells derived from Drosophila melanogaster, such as Schneider 2 cells, cell lines derived from the army worm Spodoptera frugiperda, such as Sf9 and Sf21 cells, or cells derived from the cabbage looper Trichoplusia ni, such as High Five cells), plant cell systems infected with recombinant virus expression vectors (e.g., tobacco mosaic virus) or transformed with recombinant plasmid expression vectors (e.g., Ti plasmid). Mammalian expression systems include human and non-human mammalian cells, such as rodent cells, primate cells, or human cells. Mammalian cells, such as human or non-human mammalian cells, may include primary cells, secondary, tertiary etc. cells, or may include immortalised cell lines, including clonal cell lines. Preferred animal cells can be readily maintained and transformed in tissue culture. Non-limiting example of human cells include the human HeLa (cervical cancer) cell line. Other human cell lines common in tissue culture practice include inter alia human embryonic kidney 293 cells (HEK cells), DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSY5Y (neuroblastoma), or Saos-2 cells (bone cancer). A non-limiting example of primate cells are Vero (African green monkey Chlorocebus kidney epithelial cell line) cells, and COS cells. Non-limiting examples of rodent cells are rat GH3 (pituitary tumor), CHO (Chinese hamster ovary), PC12 (pheochromocytoma) cell lines, or mouse MC3T3 (embryonic calvarium) cell line.

Any molecules, such as proteins, polypeptides or peptides as prepared herein can be suitably purified. The term “purified” with reference to molecules, peptides, polypeptides or proteins does not require absolute purity. Instead, it denotes that such molecules, peptides, polypeptides or proteins are in a discrete environment in which their abundance (conveniently expressed in terms of mass or weight or concentration) relative to other components is greater than in the starting composition or sample, e.g., in the production sample, such as in a lysate or supernatant of a recombinant host cells producing the molecule, peptide, polypeptide or protein. A discrete environment denotes a single medium, such as for example a single solution, gel, precipitate, lyophilisate, etc. Purified molecules, proteins, polypeptides or peptides may be obtained by known methods including, for example, chemical synthesis, chromatography, preparative electrophoresis, centrifugation, precipitation, affinity purification, etc. Purified molecules, peptides, polypeptides or proteins may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the non-solvent content of the discrete environment. For example, purified peptides, polypeptides or proteins may preferably constitute by weight 10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Purity of peptides, polypeptides, or proteins may be determined by HPLC, or SDS-PAGE under reducing or non-reducing conditions using Coomassie blue or, preferably, silver stain.

Any molecules, such as proteins, polypeptides or peptides as prepared herein can be suitably kept in solution in deionised water, or in deionised water with DMSO, e.g., 50% v/v DMSO in deionised water, or in an aqueous solution, or in a suitable buffer, such as in a buffer having physiological pH, or at pH between 5 and 9, more particular pH between 6 and 8, such as in neutral buffered saline, phosphate buffered saline, Tris-HCl, acetate or phosphate buffers, or in a strong chaotropic agent such as 6M urea, at concentrations of the molecules convenient for downstream use, such as without limitation between about 1 mM and about 500 mM, or between about 1 mM and about 250 mM, or between about 1 mM and about 100 mM, or between about 5 mM and about 50 mM, or between about 5 mM and about 20 mM. Alternatively, any molecules, such as proteins, polypeptides or peptides as prepared herein may be lyophilised as is generally known in the art. Storage may typically be at or below room temperature (at or below 25° C.), in certain embodiments at temperatures above 0° C. (non-cryogenic storage), such as at a temperature above 0° C. and not exceeding 25° C., or in certain embodiments cryopreservation may be preferred, at temperatures of 0° C. or lower, typically −5° C. or lower, more typically −10° C. or lower, such as −20° C. or lower, −25° C. or lower, −30° C. or lower, or even at −70° C. or lower or −80° C. or lower, or in liquid nitrogen.

Recombinant nucleic acid technology may allow not only for heterologous expression and isolation of pept-ins which are of polypeptide nature and are encoded by the nucleic acids, but may even allow to administer such pept-ins as transgenes, i.e., to administer nucleic acids (such as, for example, DNA-based or RNA-based cassettes, vectors or constructs) encoding the respective pept-ins and capable of effecting the expression of the respective pept-ins when introduced into a cell. For example, in a DNA construct a pept-in coding sequence may be operably linked to regulatory sequence(s) configured to drive the transcription and translation of the pept-in from the DNA construct, such as a promoter and a transcription terminator. In an RNA or mRNA construct a pept-in coding sequence may be included such that it can be translated by the cellular protein translation machinery. In aforementioned constructs a pept-in coding sequence will be typically preceded by an in-frame translation initiation codon and followed by a translation termination codon, to facilitate proper translation. Accordingly, wherever administration of/therapy with pept-ins as taught herein is envisaged in this specification, the administration of nucleic acids encoding those pept-ins is encompassed by the disclosure. Such administration/therapy may commonly be referred to as gene therapy. Thus all methods and uses involving the molecules of the application thus also encompass methods and uses where the molecules are provided as the nucleic acid sequence encoding them, and the molecules are expressed from the nucleic acid sequence.

Hence, also provided herein is a nucleic acid encoding any pept-in molecule as disclosed herein, where such pept-in molecule is of polypeptide nature. It is particularly envisaged that the nucleic acid sequences encode the molecules with all the features and variations described herein, mutatis mutandis. Thus, the encoded polypeptide is in essence as described herein, that is to say, the variations mentioned for the pept-in molecules that are compatible with this aspect are also envisaged as variations for the polypeptides encoded by the nucleic acid sequences.

In certain embodiments, the nucleic acid sequence is an artificial gene. Since the nucleic acid aspect is most particularly suitable in applications making use of transgenic expression, particularly envisaged embodiments may be those where the nucleic acid sequence (or the artificial gene) is fused to another moiety, particularly a moiety that increases solubility and/or stability of the gene product.

Also provided in this aspect are recombinant vectors comprising such a nucleic acid sequence encoding a molecule as herein described. These recombinant vectors are ideally suited as a vehicle to carry the nucleic acid sequence of interest inside a cell where the protein to be downregulated is expressed, and drive expression of the nucleic acid in said cell. The recombinant vector may persist as a separate entity in the cell (e.g., as a plasmid), or may be integrated into the genome of the cell. Recombinant vectors include among others plasmid vectors, binary vectors, cloning vectors, expression vectors, shuttle vectors and viral vectors. Thus, also encompassed herein are methods and uses where the molecules are provided as recombinant vectors with a nucleic acid sequence encoding the molecules, and the molecules are expressed from the nucleic acid sequence provided in the recombinant vector. Accordingly, cells are provided herein comprising a nucleic acid sequence encoding a molecule as herein described, or comprising a recombinant vector that contains a nucleic acid sequence encoding such pept-in molecule.

The molecules as taught herein are useful for therapy. Hence, an aspect provides any molecule as taught herein for use in medicine, or in other words, any molecule as taught herein for use in therapy. As discussed below, the molecules as taught herein can be formulated into pharmaceutical compositions. Therefore, any reference to the use of the molecules in therapy (or any variation of such language) also subsumes the use of pharmaceutical compositions comprising the molecules in therapy.

In particular, the molecules are intended for therapy of afflictions in which mutations at position 12 or 13 of human RAS play an important role. Accordingly, also provided is any molecule as taught herein for use in a method of treating a disease caused by or associated with a G12 or G13 mutation in human RAS protein. Further provided is a method for treating a subject in need thereof, in particularly a subject having a disease caused by or associated with a G12 or G13 mutation in human RAS protein, the method comprising administering to the subject a therapeutically effective amount of any molecule as taught herein. Further provided is use of any molecule as taught herein for the manufacture of a medicament for the treatment of a disease caused by or associated with a G12 or G13 mutation in human RAS protein. Further provided is use of any molecule as taught herein for the treatment of a disease caused by or associated with a G12 or G13 mutation in human RAS protein.

Reference to “therapy” or “treatment” broadly encompasses both curative and preventative treatments, and the terms may particularly refer to the alleviation or measurable lessening of one or more symptoms or measurable markers of a pathological condition such as a disease or disorder.

The terms encompass primary treatments as well as neo-adjuvant treatments, adjuvant treatments and adjunctive therapies. Measurable lessening includes any statistically significant decline in a measurable marker or symptom. Generally, the terms encompass both curative treatments and treatments directed to reduce symptoms and/or slow progression of the disease. The terms encompass both the therapeutic treatment of an already developed pathological condition, as well as prophylactic or preventative measures, wherein the aim is to prevent or lessen the chances of incidence of a pathological condition. In certain embodiments, the terms may relate to therapeutic treatments. In certain other embodiments, the terms may relate to preventative treatments. Treatment of a chronic pathological condition during the period of remission may also be deemed to constitute a therapeutic treatment. The term may encompass ex vivo or in vivo treatments as appropriate in the context of the present invention.

The terms “subject”, “individual” or “patient” are used interchangeably throughout this specification, and typically and preferably denote humans, but may also encompass reference to non-human animals, preferably warm-blooded animals, even more preferably non-human mammals Particularly preferred are human subjects including both genders and all age categories thereof. In other embodiments, the subject is an experimental animal or animal substitute as a disease model. The term does not denote a particular age or sex. Thus, adult and newborn subjects, as well as fetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. Appropriate therapeutically effective doses of the present molecules may be determined by a qualified physician with due regard to the nature and severity of the disease, and the age and condition of the patient. The effective amount of the molecules described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

The reference to a disease caused by or associated with a G12 or G13 mutation in human RAS protein intends to broadly encompass any disease in which the G12 or G13 RAS mutation plays at least some part in the disease, and therefore in which downregulation of the G12 mutant RAS could be of therapeutic benefit. For example, the G12 or G13 RAS mutation may be solely, or jointly with other factors such as other mutations, responsible for or contribute to the aetiology of the disease, and/or the G12 or G13 RAS mutation may be solely, or jointly with other factors such as other mutations, responsible for or contribute to the persistence, progression, worsening, resistance to other treatments or reappearance of the disease. Given the serious impacts of G12 or G13 RAS mutations on RAS activity, it may be practicably assumed that any disease typified by a G12 or G13 RAS mutation is a disease caused by or associated with the G12 or G13 RAS mutation as intended herein.

In the present context, particularly intended are G12 or G13 RAS mutations which lead to permanently or constitutively activated RAS signalling, such as G12 RAS mutations discussed elsewhere in this specification, such as in particular G12V, G12C, G12S and G12A RAS mutations, or G13V, G13C and G13S RAS mutations.

Further in the present context, particularly intended are somatic G12 or G13 RAS mutations. The term “somatic mutation” as used herein broadly refers to an acquired alteration in DNA of a subject that occurs after conception. Techniques to detect somatic RAS mutations in subjects, such as PCR amplification and sequencing or otherwise genotyping the RAS gene or the mutation-containing part thereof in a sample containing somatic cells from a subject, wherein such genetic information may where necessary or informative be compared to the subject's germline sequence variation in RAS, are well-established in the art. Considering the role RAS mutations play in neoplastic diseases, illustrative samples may include those containing tumor cells of a subject, such as without limitation, tumor tissue biopsies (e.g., primary or metastatic tumor tissue; e.g., formalin-fixed, paraffin-embedded tumor tissue or fresh-frozen tumour tissue), fine needle aspirates, blood samples (liquid' biopsies), or body exudates into which tumour cells may be shed, such as saliva, urine, stool (feces), tears, sweat, sebum, nipple aspirate, ductal lavage, cerebrospinal fluid, or lymph.

As stated earlier, gain-of-function missense mutations in RAS genes are found in about 27% of all human cancers and up to 90% in certain types of cancer, validating mutant RAS genes as very common if not the most common oncogenes driving tumor initiation and maintenance. Hence, in certain preferred embodiments, the disease is a neoplastic disease, particularly cancer.

Accordingly, also provided is any molecule as taught herein for use in a method of treating a neoplastic disease, particularly cancer, caused by or associated with a G12 or G13 mutation in human RAS protein. Further provided is a method for treating a subject in need thereof, in particular a subject having a neoplastic disease, particularly cancer, caused by or associated with a G12 or G13 mutation in human RAS protein, the method comprising administering to the subject a therapeutically effective amount of any molecule as taught herein. Further provided is use of any molecule as taught herein for the manufacture of a medicament for the treatment of a neoplastic disease, particularly cancer, caused by or associated with a G12 or G13 mutation in human RAS protein. Further provided is use of any molecule as taught herein for the treatment of a neoplastic disease, particularly cancer, caused by or associated with a G12 or G13 mutation in human RAS protein.

The term “neoplastic disease” generally refers to any disease or disorder characterised by neoplastic cell growth and proliferation, whether benign (not invading surrounding normal tissues, not forming metastases), pre-malignant (pre-cancerous), or malignant (invading adjacent tissues and capable of producing metastases). The term neoplastic disease generally includes all transformed cells and tissues and all cancerous cells and tissues. Neoplastic diseases or disorders include, but are not limited to abnormal cell growth, benign tumors, premalignant or precancerous lesions, malignant tumors, and cancer. Examples of neoplastic diseases or disorders are benign, pre-malignant, or malignant neoplasms located in any tissue or organ, such as in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the terms “tumor” or “tumor tissue” refer to an abnormal mass of tissue that results from excessive cell division. A tumor or tumor tissue comprises tumor cells which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise tumor-associated non-tumor cells, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “cancer” refers to a malignant neoplasm characterised by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). The term “metastatic” or “metastasis” generally refers to the spread of a cancer from one organ or tissue to another non-adjacent organ or tissue. The occurrence of the neoplastic disease in the other non-adjacent organ or tissue is referred to as metastasis.

Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as CNS cancer, melanoma, head and neck cancer, bone cancer, bone marrow cancer, duodenum cancer, esophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Non-melanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour.

In certain embodiments, the disease, neoplastic disease or cancer may be pancreatic ductal adenocarcinoma, colorectal adenocarcinoma, multiple myeloma, lung adenocarcinoma, skin cutaneous melanoma, uterine corpus endometrioid carcinoma, uterine carcinosarcoma, thyroid carcinoma, acute myeloid leukaemia, bladder urothelial carcinoma, gastric adenocarcinoma, cervical adenocarcinoma, head and neck squamous cell carcinoma, non-small cell lung cancer (NSCLC), or colorectal cancer.

In certain embodiments, any molecule as taught herein may be administered as the sole pharmaceutical agent (active pharmaceutical ingredient) or in combination with one or more other pharmaceutical agents where the combination causes no unacceptable adverse effects. By means of an example, two or more molecules as taught herein may be co-administered. By means of another example, one or more molecules as taught herein may be co-administered with a pharmaceutical agent that is not a molecule as envisaged herein. For example, the molecules as taught herein may be combined with known anti-cancer therapy or therapies, such as for example surgery, radiotherapy, chemotherapy, biological therapy, or combinations thereof. The term “chemotherapy” as used herein is conceived broadly and generally encompasses treatments using chemical substances or compositions. Chemotherapeutic agents may typically display cytotoxic or cytostatic effects. In certain embodiments, a chemotherapeutic agent may be an alkylating agent, a cytotoxic compound, an anti-metabolite, a plant alkaloid, a terpenoid, a topoisomerase inhibitor, or a combination thereof. The term “biological therapy” as used herein is conceived broadly and generally encompasses treatments using biological substances or compositions, such as biomolecules, or biological agents, such as viruses or cells. In certain embodiments, a biomolecule may be a peptide, polypeptide, protein, nucleic acid, or a small molecule (such as primary metabolite, secondary metabolite, or natural product), or a combination thereof. Examples of suitable biomolecules include without limitation interleukins, cytokines, anti-cytokines, tumor necrosis factor (TNF), cytokine receptors, vaccines, interferons, enzymes, therapeutic antibodies, antibody fragments, antibody-like protein scaffolds, or combinations thereof. Examples of suitable biomolecules include but are not limited to aldesleukine, alemtuzumab, atezolizumab, bevacizumab, blinatumomab, brentuximab vedotine, catumaxomab, cetuximab, daratumumab, denileukin diftitox, denosumab, dinutuximab, elotuzumab, gemtuzumab ozogamicin, ⁹⁰Y-ibritumomab tiuxetan, idarucizumab, interferon A, ipilimumab, necitumumab, nivolumab, obinutuzumab, ofatumumab, olaratumab, panitumumab, pembrolizumab, ramucirumab, rituximab, tasonermin, ¹³¹I-tositumomab, trastuzumab, Ado-trastuzumab emtansine, and combinations thereof Examples of suitable oncolytic viruses include but are not limited to talimogene laherparepvec. Further categories of anti-cancer therapy include inter alia hormone therapy (endocrine therapy), immunotherapy, and stem cell therapy, which are commonly considered as subsumed within biological therapies. Examples of suitable hormone therapies include but are not limited to tamoxifen; aromatase inhibitors, such as atanastrozole, exemestane, letrozole, and combinations thereof; luteinizing hormone blockers such as goserelin, leuprorelin, triptorelin, and combinations thereof; anti-androgens, such as bicalutamide, cyproterone acetate, flutamide, and combinations thereof; gonadotrophin releasing hormone blockers, such as degarelix; progesterone treatments, such as medroxyprogesterone acetate, megestrol, and combinations thereof; and combinations thereof. The term “immunotherapy” broadly encompasses any treatment that modulates a subject's immune system. In particular, the term comprises any treatment that modulates an immune response, such as a humoral immune response, a cell-mediated immune response, or both.

Immunotherapy comprises cell-based immunotherapy in which immune cells, such as T cells and/or dendritic cells, are transferred into the patient. The term also comprises an administration of substances or compositions, such as chemical compounds and/or biomolecules (e.g., antibodies, antigens, interleukins, cytokines, or combinations thereof), that modulate a subject's immune system. Examples of cancer immunotherapy include without limitation treatments employing monoclonal antibodies, for example Fc-engineered monoclonal antibodies against proteins expressed by tumor cells, immune checkpoint inhibitors, prophylactic or therapeutic cancer vaccines, adoptive cell therapy, and combinations thereof. Examples of immune checkpoint targets for inhibition include without limitation PD-1 (examples of PD-1 inhibitors include without limitation pembrolizumab, nivolumab, and combinations thereof), CTLA-4 (examples of CTLA-4 inhibitors include without limitation ipilimumab, tremelimumab, and combinations thereof), PD-L1 (examples of PD-L1 inhibitors include without limitation atezolizumab), LAG3, B7-H3 (CD276), B7-H4, TIM-3, BTLA, A2aR, killer cell immunoglobulin-like receptors (KIRs), IDO, and combinations thereof Another approach to therapeutic anti-cancer vaccination includes dendritic cell vaccines. The term broadly encompasses vaccines comprising dendritic cells which are loaded with antigen(s) against which an immune reaction is desired. Adoptive cell therapy (ACT) can refer to the transfer of cells, most commonly immune-derived cells, such as in particular cytotoxic T cells (CTLs), back into the same patient or into a new recipient host with the goal of transferring the immunologic functionality and characteristics into the new host. If possible, use of autologous cells helps the recipient by minimizing tissue rejection and graft vs. host disease issues. Various strategies may for example be employed to genetically modify T cells by altering the specificity of the T cell receptor (TCR) for example by introducing new TCR α and β chains with selected peptide specificity. Alternatively, chimeric antigen receptors (CARs) may be used in order to generate immunoresponsive cells, such as T cells, specific for selected targets, such as malignant cells, with a wide variety of receptor chimera constructs having been described. Examples of CAR constructs include without limitation 1) CARs consisting of a single-chain variable fragment of an antibody specific for an antigen, for example comprising a V_{L} linked to a V_{H} of a specific antibody, linked by a flexible linker, for example by a CD8α hinge domain and a CD8α transmembrane domain, to the transmembrane and intracellular signaling domains of either CD3ζ or FcRγ; and 2) CARs further incorporating the intracellular domains of one or more costimulatory molecules, such as CD28, OX40 (CD134), or 4-1BB (CD137) within the endodomain, or even including combinations of such costimulatory endodomains. Stem cell therapies in cancer commonly aim to replace bone marrow stem cells destroyed by radiation therapy and/or chemotherapy, and include without limitation autologous, syngeneic, or allogeneic stem cell transplantation. The stem cells, in particular hematopoietic stem cells, are typically obtained from bone marrow, peripheral blood or umbilical cord blood. Details of administration routes, doses, and treatment regimens of anti-cancer agents are known in the art, for example as described in “Cancer Clinical Pharmacology” (2005) ed. By Jan H. M. Schellens, Howard L. McLeod and David R. Newell, Oxford University Press. In certain embodiments, a combination therapy with any molecule as taught herein with one or more of a MEK inhibitor (e.g. selumetinib or trametinib), a SHP2 inhibitor (e.g., TNO155), an mTOR inhibitor (e.g., rapamycin or a rapamycin derivative (“rapalog”), including sirolimus, temsirolimus (CCI-779), temsirolimus (CCI-779), everolimus (RAD001), and ridaforolimus (AP-23573)) is envisaged. Active components of any combination therapy may be admixed or may be physically separated, and may be administered simultaneously or sequentially in any order.

Any molecule as taught herein may be administered to subjects in any suitable or operable form or format.

For example, the reference to the molecule as intended herein may encompass a given therapeutically useful compound as well as any pharmaceutically acceptable forms of such compound, such as any addition salts, hydrates or solvates of the compound. The term “pharmaceutically acceptable” as used herein inter alia in connection with salts, hydrates, solvates and excipients, is consistent with the art and means compatible with the other ingredients of a pharmaceutical composition and not deleterious to the recipient thereof. Pharmaceutically acceptable acid and base addition salts are meant to comprise the therapeutically active non-toxic acid and base addition salt forms which the compound is able to form. The pharmaceutically acceptable acid addition salts can conveniently be obtained by treating the base form of a compound with an appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form. A compound containing an acidic proton may also be converted into its non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, aluminum salts, zinc salts, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form. The term solvate comprises the hydrates and solvent addition forms which the compound is able to form, as well as the salts thereof. Examples of such forms are, e.g., hydrates, alcoholates and the like.

For example, the molecule may be a part of a composition. The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. By means of an example, a composition may comprise any molecule as taught herein in combination with one or more other substances. For example, a composition may be obtained by combining, such as admixing, the molecule as taught herein with said one or more other substances. In certain embodiments, the present compositions may be configured as pharmaceutical compositions. Pharmaceutical compositions typically comprise one or more pharmacologically active ingredients (chemically and/or biologically active materials having one or more pharmacological effects) and one or more pharmaceutically acceptable carriers. Compositions as typically used herein may be liquid, semisolid or solid, and may include solutions or dispersions.

Hence, a further aspect provides a pharmaceutical composition comprising any molecule as taught herein. The terms “pharmaceutical composition” and “pharmaceutical formulation” may be used interchangeably. The pharmaceutical compositions as taught herein may comprise in addition to the one or more actives, one or more pharmaceutically or acceptable carriers. Suitable pharmaceutical excipients depend on the dosage form and identities of the active ingredients and can be selected by the skilled person (e.g., by reference to the Handbook of Pharmaceutical Excipients 7^{th} Edition 2012, eds. Rowe et al.).

As used herein, the terms “carrier” or “excipient” are used interchangeably and broadly include any and all solvents, diluents, buffers (such as, e.g., neutral buffered saline, phosphate buffered saline, or optionally Tris-HCl, acetate or phosphate buffers), solubilisers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical and cosmetic compositions is well known in the art. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the actives. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability.

The pharmaceutical formulations may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art. Liposomes are artificial membrane vesicles which are useful as delivery vehicles in vitro and in vivo. These formulations may have net cationic, anionic or neutral charge characteristics and are useful characteristics with in vitro, in vivo and ex vivo delivery methods. It has been shown that large unilamellar vesicles (LUV), which range in size from 0.2-4.0 PHI.m can encapsulate a substantial percentage of an aqueous buffer containing large macromolecules. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the agent as disclosed herein in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the agent as disclosed herein with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the agents as taught herein or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution, PBS, Ringer's solution, dextrose solution, or Hank's solution, or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agents and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognise that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The dosage or amount of the molecules as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the molecule as taught herein can be first administered at different dosing regimens. Typically, levels of the molecule in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the molecules as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

Without limitation, depending on the type and severity of the disease, a typical dosage (e.g., a typical daily dosage or a typical intermittent dosage, e.g., a typical dosage for every two days, every three days, every four days, every five days, every six days, every week, every 1.5 weeks, every two weeks, every three weeks, every month, or other) of the molecules as taught herein may range from about 10 μg/kg to about 100 mg/kg body weight of the subject, per dose, depending on the factors mentioned above, e.g., may range from about 100 μg/kg to about 100 mg/kg body weight of the subject, per dose, or from about 200 μg/kg to about 75 mg/kg body weight of the subject, per dose, or from about 500 μg/kg to about 50 mg/kg body weight of the subject, per dose, or from about 1 mg/kg to about 25 mg/kg body weight of the subject, per dose, or from about 1 mg/kg to about 10 mg/kg body weight of the subject, per dose, e.g., may be about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1.0 mg/kg, about 2.0 mg/kg, about 5.0 mg/kg, about 10 mg/kg, about 15 mg/kg, about 20 mg/kg, about 30 mg/kg, about 40 mg/kg, about 50 mg/kg, about 75 mg/kg, or about 100 mg/kg body weight of the subject, per dose.

In particular embodiments, the molecule as taught herein is administered using a sustained delivery system, such as a (partly) implanted sustained delivery system. Skilled person will understand that such a sustained delivery system may comprise a reservoir for holding the agent as taught herein, a pump and infusion means (e.g., a tubing system).

Hence, the present application also provides aspects and embodiments as set forth in the following Statements:

Statement 1. A non-naturally occurring molecule configured to form an intermolecular beta-sheet with a human RAS protein mutated at position 12 and substantially not with wild-type human RAS protein.

Statement 2. The molecule according to Statement 1, wherein the mutant human RAS protein is a G12V, G12C, G12A, or G12S mutant human RAS protein, preferably a G12V mutant human RAS protein.

Statement 3. The molecule according to Statement 1 or 2, wherein the RAS protein is KRAS, NRAS or HRAS protein, preferably KRAS protein.

Statement 4. The molecule according to any one of Statements 1 to 3, wherein the intermolecular beta-sheet involves the amino acid at position 12 of the mutant human RAS protein.

Statement 5. The molecule according to Statement 4, wherein the intermolecular beta-sheet involves:
a) a portion of or the whole of the amino acid sequence TEYKLVVVGAVGVG (SEQ ID NO: 2) in the G12V mutant human RAS protein; or
b) a portion of or the whole of the amino acid sequence TEYKLVVVGACGVG (SEQ ID NO: 6) or preferably TEYKLVVVGACGV (SEQ ID NO: 3) in the G12C mutant human RAS protein; or
c) a portion of or the whole of the amino acid sequence TEYKLVVVGAAGVG (SEQ ID NO: 7) or preferably TEYKLVVVGAAGV (SEQ ID NO: 4) in the G12A mutant human RAS protein; or
d) a portion of or the whole of the amino acid sequence TEYKLVVVGASGVG (SEQ ID NO: 8) or preferably TEYKLVVVGASGV (SEQ ID NO: 9) or more preferably TEYKLVVVGASG (SEQ ID NO: 5) in the G12S mutant human RAS protein.

Statement 6. The molecule according to any one of Statements 1 to 5, wherein the molecule is able to decrease the solubility or to induce the aggregation or inclusion body formation of the human RAS protein mutated at position 12.

Statement 7. The molecule according to any one of Statements 1 to 6, wherein the molecule comprises an amino acid stretch which participates in the intermolecular beta-sheet.

Statement 8. The molecule according to Statement 7, wherein the amino acid stretch comprises at least 6 contiguous amino acids of the amino acid sequence:

a)
(SEQ ID NO: 2)
TEYKLVVVGAVGVG;
or
b)
(SEQ ID NO: 6)
TEYKLVVVGACGVG
or preferably
(SEQ ID NO: 3)
TEYKLVVVGACGV;
or
c)
(SEQ ID NO: 7)
TEYKLVWGAAGVG
or preferably
(SEQ ID NO: 4)
TEYKLVVVGAAGV;
or
d)
(SEQ ID NO: 8)
TEYKLVVVGASGVG
or preferably
(SEQ ID NO: 9)
TEYKLVVVGASGV
or more preferably
(SEQ ID NO: 5)
TEYKLVVVGASG;

including the amino acid at position 11 of the respective sequences.

Statement 9. The molecule according to Statement 7 or 8, wherein the molecule comprises the amino acid stretch VVVGAV (SEQ ID NO: 10), LVVVGAV (SEQ ID NO: 11), VVVGAVG (SEQ ID NO: 12) or VVVGAVGVG (SEQ ID NO: 13).

Statement 10. The molecule according to any one of Statements 7 to 9, wherein the amino acid stretch comprises one or more D-amino acids and/or analogues of one or more of its amino acids.

Statement 11. The molecule according to any one of Statements 7 to 10, wherein the molecule comprises two or more, preferably two, said amino acid stretches, which are identical or different.

Statement 12. The molecule according to any one of Statements 7 to 11, wherein the amino acid stretch or stretches are each independently flanked, on each end independently, by one or more amino acids that display low beta-sheet forming potential or a propensity to disrupt beta-sheets.

Statement 13. The molecule according to any one of Statements 7 to 12, wherein the molecule comprises, consists essentially of or consists of the structure:
a) NGK1-P1-CGK1,
b) NGK1 -P1-CGK1-Z 1-NGK2-P2-CGK2,
c) NGK1 -P1-CGK1-Z 1-NGK2-P2-CGK2-Z2-NGK3-P3 -CGK3, or
d) NGK1 -P1-CGK1-Z 1-NGK2-P2-CGK2-Z2-NGK3 -P3-CGK3 -Z3-NGK4-P4-CGK4,
   wherein:
P1 to P4 each independently denote an amino acid stretch as defined in any one of Statements 7 to 10,
NGK1 to NGK4 and CGK1 to CGK4 each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof, and
Z1 to Z3 each independently denote a direct bond or preferably a linker. Statement 14. The molecule according to Statement 13, wherein:
NGK1 to NGK4 and CGK1 to CGK4 is each independently 1 to 2 contiguous amino acids selected from the group consisting of R, K, A, and D, D-isomers and/or analogues thereof, and combinations thereof, preferably NGK1 to NGK4 and CGK1 to CGK4 is each independently 1 to 2 contiguous amino acids selected from the group consisting of R, K, and D, D-isomers and/or analogues thereof, and combinations thereof, such as wherein NGK1 to NGK4 and CGK1 to CGK4 is each independently K, R, D, A or KK, preferably each independently K, R, D or KK; and/or
each linker is independently selected from a stretch of between 1 and 10 units, preferably between 1 and 5 units, wherein a unit is each independently an amino acid or PEG, such as wherein each linker is independently GS, PP, AS, SA, GF, FF, or GSGS (SEQ ID NO: 14), or D-isomers and/or analogues thereof, preferably each linker is independently GS, PP or GSGS (SEQ ID NO: 14), preferably GS, or D-isomers and/or analogues thereof.

Statement 15. The molecule according to Statement 13 or 14, wherein the molecules comprises, consists essentially of or consists of a peptide of the amino acid sequence:

a)
(SEQ ID NO: 15)
KVVVGAVKGSKVVVGAVK ;
or
b)
(SEQ ID NO: 16)
KLVVVGAVKGSKLVVVGAVK ;
or
c)
(SEQ ID NO: 17)
KVVVGAVGKGSKVVVGAVGK ;
or
d)
(SEQ ID NO: 18)
KVVVGAVGVGKGSKVVVGAVGVGK ;
or
e)
(SEQ ID NO: 160)
[Dap]LSVFAIKGSKLSVFAI[Dap];
or
f)
(SEQ ID NO: 161)
[Dap]VVVGAVKGSKVVVGAV[Dap];
or
g)
(SEQ ID NO: 162)
[Dap]VVVGAVGKGSKVVVGAVG[Dap];
or
h)
(SEQ ID NO: 163)
[Dap]VVVGAVGVGKGSKVVVGAVGVG[Dap];
or
i)
(SEQ ID NO: 164)
[Cit]VVVGAVKGSKVVVGAVK;
or
j)
(SEQ ID NO: 165)
KVVVGAV[Cit]GSKVVVGAVK;
or
k)
(SEQ ID NO: 166)
AVVVGAVKGSKVVVGAVK;
or
l)
(SEQ ID NO: 167)
KVVVGAVAGSKVVVGAVK;
or
m)
(SEQ ID NO: 168)
KVVVGAVKGSAVVVGAVK;
or
n)
(SEQ ID NO: 169)
KVVVGAVKGSKVVVGAVA;
or
o)
(SEQ ID NO: 170)
AVVVGAVKGSAVVVGAVK;
or
p)
(SEQ ID NO: 171)
KVVVGAVAGSKVVVGAVA;
or
q)
(SEQ ID NO: 172)
AVVVGAVAGSKVVVGAVK;
or
r)
(SEQ ID NO: 173)
KVVVGAVKASKVVVGAVK;
or
s)
(SEQ ID NO: 174)
KVVVGAVKGAKVVVGAVK;
or
t)
(SEQ ID NO: 175)
KVVVGAVGKGFKVVVGAVGK;
or
u)
(SEQ ID NO: 176)
KVVVGAVGKFFKVVVGAVGK;
or
v)
(SEQ ID NO: 178)
KVVVGAVGVGKKVVVGAVGVGK;

optionally wherein the amino acid sequence comprises one or more D-amino acids and/or analogues of one or more of its amino acids, optionally wherein the N-terminal amino acid is acetylated and/or the C-terminal amino acid is amidated ‘[Dap]’ denotes diaminopimelic acid, ‘[Cit]’ denotes citrulline).

Statement 16. The molecule according to any one of Statements 1 to 15, which comprises a detectable label, a moiety that allows for isolation of the molecule, a moiety increasing the stability or half-life of the molecule, a moiety increasing the solubility of the molecule, a moiety increasing the cellular uptake of the molecule, and/or a moiety effecting targeting of the molecule to cells.

Statement 17. The molecule according to any one of Statements 1 to 16 for use in medicine.

Statement 17.′ A nucleic acid encoding the molecule according to any one of Statements 1 to 16, wherein the molecule is a polypeptide, for use in medicine.

Statement 18. The molecule according to any one of Statements 1 to 16 for use in a method of treating a disease caused by or associated with a mutation at position 12 in human RAS protein.

Statement 18′. A nucleic acid encoding the molecule according to any one of Statements 1 to 16, wherein the molecule is a polypeptide, for use in a method of treating a disease caused by or associated with a mutation at position 12 in human RAS protein.

Statement 19. The molecule or nucleic acid for use according to Statement 18 or 18′, wherein the disease is a neoplastic disease, particularly cancer.

Statement 20. The molecule or nucleic acid for use according to Statement 18, 18′ or 19, wherein the disease is pancreatic ductal adenocarcinoma, colorectal adenocarcinoma, multiple myeloma, lung adenocarcinoma, skin cutaneous melanoma, uterine corpus endometrioid carcinoma, uterine carcinosarcoma, thyroid carcinoma, acute myeloid leukaemia, bladder urothelial carcinoma, gastric adenocarcinoma, cervical adenocarcinoma, head and neck squamous cell carcinoma, non-small cell lung cancer (NSCLC), or colorectal cancer.

Statement 21. A pharmaceutical composition comprising the molecule according to any one of Statements 1 to 16.

Statement 21′. A pharmaceutical composition comprising a nucleic acid encoding the molecule according to any one of Statements 1 to 16, wherein the molecule is a polypeptide.

The present application also provides aspects and embodiments as set forth in the following Statements:

Statement 1*. A non-naturally occurring molecule configured to form an intermolecular beta-sheet with a human RAS protein mutated at position 13 and substantially not with wild-type human RAS protein.

Statement 2*. The molecule according to Statement 1*, wherein the mutant human RAS protein is a G13V, G13C, or G13S mutant human RAS protein, preferably a G13V mutant human RAS protein.

Statement 3*. The molecule according to Statement 1* or 2*, wherein the RAS protein is KRAS, NRAS or HRAS protein, preferably KRAS protein.

Statement 4*. The molecule according to any one of Statements 1* to 3*, wherein the intermolecular beta-sheet involves the amino acid at position 13 of the mutant human RAS protein.

Statement 5*. The molecule according to Statement 4*, wherein the intermolecular beta-sheet involves:
a) a portion of or the whole of the amino acid sequence TEYKLVVVGAGVVG (SEQ ID NO: 82) in the G13V mutant human RAS protein; or
b) a portion of or the whole of the amino acid sequence TEYKLVVVGAGCVG (SEQ ID NO: 84) or preferably TEYKLVVVGAGCV (SEQ ID NO: 81) in the G13C mutant human RAS protein; or
c) a portion of or the whole of the amino acid sequence TEYKLVVVGAGSVG (SEQ ID NO: 85) or preferably TEYKLVVVGAGSV (SEQ ID NO: 86) or more preferably TEYKLVVVGAGS (SEQ ID NO: 83) in the G13 S mutant human RAS protein.

Statement 6*. The molecule according to any one of Statements 1* to 5*, wherein the molecule is able to decrease the solubility or to induce the aggregation or inclusion body formation of the human RAS protein mutated at position 13.

Statement 7*. The molecule according to any one of Statements 1* to 6*, wherein the molecule comprises an amino acid stretch which participates in the intermolecular beta-sheet.

Statement 8*. The molecule according to Statement 7*, wherein the amino acid stretch comprises at least 6 contiguous amino acids of the amino acid sequence:

a)
(SEQ ID NO: 82)
TEYKLVVVGAGVVG;
or
b)
(SEQ ID NO: 84)
TEYKLVVVGAGCVG
or preferably
(SEQ ID NO: 81)
TEYKLVVVGAGCV;
or
c)
(SEQ ID NO: 85)
TEYKLVVVGAGSVG
or preferably
(SEQ ID NO: 86)
TEYKLVVVGAGSV
or
more preferably
(SEQ ID NO: 83)
TEYKLVVVGAGS;

including the amino acid at position 12 of the respective sequences.

Statement 9*. The molecule according to Statement 7* or 8*, wherein the molecule comprises the amino acid stretch VVVGAGV (SEQ ID NO: 99), LVVVGAGV (SEQ ID NO: 100), VVVGAGVV (SEQ ID NO: 101), or VVVGAGVVG (SEQ ID NO: 102).

Statement 10*. The molecule according to any one of Statements 7* to 9*, wherein the amino acid stretch comprises one or more D-amino acids and/or analogues of one or more of its amino acids.

Statement 11*. The molecule according to any one of Statements 7* to 10*, wherein the molecule comprises two or more, preferably two, said amino acid stretches, which are identical or different.

Statement 12*. The molecule according to any one of Statements 7* to 11*, wherein the amino acid stretch or stretches are each independently flanked, on each end independently, by one or more amino acids that display low beta-sheet forming potential or a propensity to disrupt beta-sheets.

Statement 13*. The molecule according to any one of Statements 7* to 12*, wherein the molecule comprises, consists essentially of or consists of the structure:
a) NGK1-P1-CGK1,
b) NGK1 -P1-CGK1-Z1-NGK2-P2-CGK2,
c) NGK1-P1-CGK1-Z1-NGK2-P2-CGK2-Z2-NGK3-P3-CGK3, or
d) NGK1 -P1-CGK1-Z 1-NGK2-P2-CGK2-Z2-NGK3 -P3-CGK3 -Z3-NGK4-P4-CGK4,
   wherein:
P1 to P4 each independently denote an amino acid stretch as defined in any one of Statements 7* to 10*,
NGK1 to NGK4 and CGK1 to CGK4 each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof, and
Z1 to Z3 each independently denote a linker.

Statement 14. The molecule according to Statement 13, wherein:
NGK1 to NGK4 and CGK1 to CGK4 is each independently 1 to 2 contiguous amino acids selected from the group consisting of R, K, and D, D-isomers and/or analogues thereof, and combinations thereof, such as wherein NGK1 to NGK4 and CGK1 to CGK4 is each independently K, R, D or KK; and/or
each linker is independently selected from a stretch of between 1 and 10 units, preferably between 1 and 5 units, wherein a unit is each independently an amino acid or PEG, such as wherein each linker is independently GS, PP or GSGS (SEQ ID NO: 14), preferably GS, or D-isomers and/or analogues thereof.

Statement 15*. The molecule according to Statement 13* or 14*, wherein the molecules comprises, consists essentially of or consists of a peptide of the amino acid sequence:

a)
(SEQ ID NO: 128)
KVVVGAGVKGSKVVVGAGVK;
or
b)
(SEQ ID NO: 129)
KLVVVGAGVKGSKLVVVGAGVK;
or
c)
(SEQ ID NO: 130)
KVVVGAGVVKGSKVVVGAGVVK;
or
d)
(SEQ ID NO: 131)
KVVVGAGVVGKGSKVVVGAGVVGK;

optionally wherein the amino acid sequence comprises one or more D-amino acids and/or analogues of one or more of its amino acids, optionally wherein the N-terminal amino acid is acetylated and/or the C-terminal amino acid is amidated.

Statement 16*. The molecule according to any one of Statements 1* to 15*, which comprises a detectable label, a moiety that allows for isolation of the molecule, a moiety increasing the stability or half-life of the molecule, a moiety increasing the solubility of the molecule, a moiety increasing the cellular uptake of the molecule, and/or a moiety effecting targeting of the molecule to cells.

Statement 17*. The molecule according to any one of Statements 1* to 16*, or, if the molecule is a polypeptide a nucleic acid encoding the molecule, for use in medicine.

Statement 17*′. A nucleic acid encoding the molecule according to any one of Statements 1* to 16*, wherein the molecule is a polypeptide, for use in medicine.

Statement 18*. The molecule according to any one of Statements 1* to 16* for use in a method of treating a disease caused by or associated with a mutation at position 13 in human RAS protein.

Statement 18*′. A nucleic acid encoding the molecule according to any one of Statements 1* to 16*, wherein the molecule is a polypeptide, for use in a method of treating a disease caused by or associated with a mutation at position 12 in human RAS protein.

Statement 19*. The molecule or nucleic acid for use according to Statement 18* or 18*′, wherein the disease is a neoplastic disease, particularly cancer.

Statement 20*. The molecule or nucleic acid for use according to Statement 18*, 18*′ or 19*, wherein the disease is pancreatic ductal adenocarcinoma, colorectal adenocarcinoma, multiple myeloma, lung adenocarcinoma, skin cutaneous melanoma, uterine corpus endometrioid carcinoma, uterine carcinosarcoma, thyroid carcinoma, acute myeloid leukaemia, bladder urothelial carcinoma, gastric adenocarcinoma, cervical adenocarcinoma, head and neck squamous cell carcinoma, non-small cell lung cancer (NSCLC), or colorectal cancer.

Statement 21*. A pharmaceutical composition comprising the molecule according to any one of Statements 1* to 16*.

Statement 21*′. A pharmaceutical composition comprising a nucleic acid encoding the molecule according to any one of Statements 1* to 16*, wherein the molecule is a polypeptide.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Materials and Methods Used in Examples 1-7

### Design of RAS-Specific Aggregating Molecules (‘Pept-Ins’)

Protein sequences for RAS family member proteins were obtained from UniProt (entries: P01116 (KRAS), P01112 (HRAS) and P01111 (NRAS)) (Nucleic Acid Res. 47 (2008) 36, D190-5). Protein sequences were analyzed using the TANGO algorithm (Fernandez-Escamilla et al. 2004, supra) to identify aggregation prone regions (APRs). To this end, the following settings were used: Temperature=298K, pH=7.5, Ionic Strength=0.10 M and a cutoff on the TANGO score of 1 per residue. To assess the impact of prevalent G12 and G13 mutations on the TANGO profile, we used a sequence fragment of 19 amino acids (1-19) containing the affected APR. This sequence fragment is 100% conserved between KRAS, HRAS and NRAS, such that the outcome applies to all RAS isoforms. Mutations were introduced manually, and sequences were analyzed using the TANGO algorithm as described above.

Based on the TANGO output using both RAS wild-type and RAS G12V sequences, we generated all possible APR windows between 6 and 10 amino acids using a sliding window approach. The resulting sequence windows were cross-compared against the full human proteome and only sequences with unique exact match with RAS proteins were retained for molecule (henceforth, ‘pept-in’) design.

### Peptide Synthesis and Purification

Solid Phase Peptide Synthesis

Peptide synthesis was performed on a Symphony X peptide synthesizer (Gyros Protein Technologies) at a 50 or 100 μmol scale. Rink amide low loading resin (100-200 mesh), 0-(1H-6- chlorobenzotriazole-1-yl)-1,1,3,3-tetramethyluronium hexafluorophosphate (HCTU) and diethyl ether were purchased from Novabiochem/Merck. Fmoc protected amino acids (AA) and trifluoroacetic acid (TFA) were purchased from Fluorochem. N,N-Dimethylformamide (DMF), 20% piperidine in DMF solution, N,N-Diisopropylethylamine (DIPEA), triisopropylsilane (TIS) and dithiothreitol (DTT) were purchased from Sigma-Aldrich. Dichloromethane (DCM) was purchased from Acros Organics. Elongation of the desired sequences were performed by repeated cycles of Fmoc removal and coupling of amino acids (see Table 10 below for scale-depending volumes and concentrations). First, resin was swollen for 2×10 minutes in DMF. The Fmoc protecting group was next removed by exposure to a solution of 20% piperidine in DMF for 2×5 minutes using. Resin was then washed with DMF and coupling was carried out using 4 eq. AA, 4 eq. HCTU and 16 eq. DIPEA in DMF for 30 min. Resin was washed with DMF prior to next cycle. Extended Fmoc removal (2×15) minutes and double couplings (2×30 minutes) were performed from the 1^{st} AA of the second APR until the end of the desired sequence. Resin was then washed several times with DMF, DCM and then dried for 2×10 minutes. Peptide was finally cleaved from dried resin using a TFA solution containing 2.5% ultrapure water; 2.5% TIS and 2.5% DTT for 2 hours. The peptide solution was then precipitated in cold diethyl ether (35 mL for 5 mL of TFA solution) and centrifuged; liquid phase was then discarded, and peptide pellet was washed with 15 mL diethyl ether. After centrifugation, the pellet was air dried for 30 min and then dissolved in 10 mL of a water/acetonitrile solution (1:1), frozen and freeze-dried on a lyophilizer overnight to afford peptide as crude powder.

TABLE 10

| | | | |
|---|---|---|---|
| Single | scale (μmol) | 50 | 100 |
| coupling | | | |
| Fmoc | 20% piperidine in | 2 | 3 |
| removal | DMF (mL) | | |
| | Large DMF wash | 6 | 6 |
| | (mL) | | |
| | DMF wash (mL) × 4 | 2 | 4 |
| Coupling | AA (mL) | 1 | 1 |
| | HCTU solution (mL) | 1 | 1 |
| | Base (mL) | 1 | 1 |
| | DMF wash (mL) × 5 | 2 | 4 |
| | Concentration (M) | 0.2/0.19/0.8 | 0.4/0.38/1.6 |
| | AA/HCTU/Base (eq.) | 4/4/16 | 4/4/16 |
| Cleavage | scale (μmol) | 50 | 100 |
| | 2 h TFA reaction (mL) | 2.5 | 5 |
| | 1st TFA wash (mL) | 2.5 | 2.5 |
| | 2nd TFA wash (mL) | 0 | 2.5 |

Peptide Purification

Crude peptides were purified via reverse phase preparative HPLC on a Gilson system equipped with a 322 Pump, a 159 UV-vis detector and a GX281 collector using a C18 column from Phenomenex (5 μm 110 Å 250×21.2 mm, ref 006-4435-P0-AX). HPLC grade water and acetonitrile were purchased from VWR and TFA was purchased from Fluorochem. Guanidine hydrochloride (Gu) was purchased from Sigma Aldrich; dimethyl sulfoxide (DMSO) and acetic acid were purchased from Merck. Solvent A is water+0.1% TFA and solvent B is acetonitrile+0.1% TFA. Crude powder was dissolved at 20 mg/mL in DMSO, vortexed and sonicated; the solution was then diluted by a factor of 10 with Gu+10% acetic acid in water and finally filtrated on a 0.22 μm cellulose acetate filter (from Merck). Peptide solution was then purified at a 30 mL/min flow using a gradient consisting of a flat time of 7 minutes at 15% B, elution from 15% B to 45% B in 10 minutes followed by a wash of the column using 95% B for 2 minutes and an equilibration at 15% B for 6 minutes. Fractions were then analyzed by MALDI mass spectrometry. Pure fractions were pulled together in a glass vial, frozen and lyophilized over at least 2 days. Pure peptide was finally analyzed by LCMS for quality control validation using 90% purity both by UV and MS signal as threshold.

### Cellular Potency Screening

Cell lines used in this application and are listed in Table 11 below:

TABLE 11

| Cell line | Supplier | Cat N^{o} |
|---|---|---|
| A-427 | ATCC | HTB-53 |
| A-549 | ATCC | CCL-185 |
| Capan-1 | CLS | 300143 |
| HCT116 | BPS Bioscience | 60520 |
| LCLC-97-TM-1 | CLS | 300409 |
| MIAPACA-2 | ATCC | CRL-1420 |
| NCI-H1299 | ATCC | CRL-5803 |
| NCI-H358 | ATCC | CRL-5807 |
| NCI-H441 | ATCC | HTB-174 |
| NCI-H727 | ATCC | CRL-5815 |
| PA-TU-8988T | DSMZ | ACC 162 |
| PANC-1 | ATCC | CRL-1469 |

DSMZ: Leibniz Institute DSMZ-German Collection of Microorganisms and Cell Cultures, Inhoffenstr. 7B, D-38124 Braunschweig Germany.

CLS: CLS Cell Lines Service, Dr.Eckener-Str. 8, D-69214 Eppelheim, Germany (www.https://clsgmbh.de/).

BPS Bioscience, 6042 Cornerstone Court West, Suite B, San Diego, Calif. 92121, United States (www.bpsbioscience.com).

Human tumor cell lines were obtained from ATCC (i.e. NCI-H441 (HBT-174^{TH}), NCI-H1299 (CRL-5803TM), NCI-H358 (CRL-5807TM), NCI-H727 (CRL-5815TM), A-427 (HTB-53TM),

PANC-1 (CRL-1469TM), HCT-116 (CCL-247TM), and MIAPaCa-2 (CRL-1420TM)), CLS Cell Line Service GmbH (i.e. Capan-1 (300143), and LCLC-97TM1 (300409)), or Leibniz-Institut DSMZ (i.e. PA-TU-8998T (ACC 162)). Mouse embryonic fibroblasts expressing a single RAS isoform (referred to as ‘RASless MEFs’) were obtained from the Frederick National Laboratory of the National Cancer Institute, Frederick, Md., USA. All cell lines were maintained according to the provider's instructions.

Adherent Viability Assays

For the single-dose viability screen on adherent cells, 4000 cells were seeded per well in black μclear® Cellstar® F-bottom 96-well plates (Greiner) in 100 μL full growth medium. The day after seeding, growth medium was replaced with full growth medium containing the indicated pept-in at a fixed final dose of 25 μM. Technical duplicates were included for all experimental pept-in conditions. 2 and 4 days after treatment viability was assessed using the CellTiter Blue reagent (Promega) according to the manufacturer's instructions, with the following adaptation: CellTiter Blue reagent was diluted 1 in 2 in PBS. Readout was performed on a Clariostar plate reader (BMG). Dose-response assays were performed with the following adaptations: pept-ins were tested in dose-response using a 1 in 2 dilution series with 50 μM being the highest final concentration used. Furthermore, a single viability read-out was performed 3 days after treatment using the Celltiter Glo reagent (Promega) according to the manufacturer's instructions, with the following adaptation: CellTiter Glo reagent was diluted 1 in 4 in PBS.

All test plates contained multiple normal growth and vehicle controls as well as a duplicate of a dose-response of the positive control compound SAH-SOS-1A (CAS no. 1652561-87-9).

Spheroid Viability Assays

For the single-dose viability screen on spheroid cultures, 1000 cells were seeded per well in black Ultra-Low Attachment (ULA) round-bottom 96-well plates (Corning) in 75 μL full growth medium. The day after seeding, spheroids were treated by addition of 50μ1 of full growth medium containing the indicated test compounds so that the final concentration after adding was 25 μM. Technical duplicates were included for all experimental pept-in conditions 5 days after treatment viability was assessed using the CellTiter Glo 3D reagent (Promega) according to the manufacturer's instructions, with the following adaptation: 80 μL of reagent was added per well. Readout was performed on a Clariostar plate reader (BMG). For dose-response assays using RASless MEFs, cells were seeded at 1000 (G12V and G12C) or 2000 (wild-type and BRAF V600E) in Matrigel-containing medium, in order to obtain equally viable spheroids at start of treatment, 24 hrs later. Dose-response assays were performed with the following adaptation: pept-ins were tested in dose-response using a 1 in 2 dilution series with 50 μM being the highest final concentration.

All test plates contained multiple normal growth and vehicle controls as well as a duplicate of a dose-response of the positive control compound SAH-SOS-1A (Merck).

### Tinctorial In Vitro Aggregation Assays

Tinctorial aggregation assays were performed using the amyloid-sensor dyes Thioflavin T (ThT) and pentameric formyl thiophene acetic acid (p-FTAA). Pept-ins were diluted from a 5 mM stock solution in 6M Urea in PBS to a final concentration of 10004. Measurements were performed in black half-area 96-well plates at 37° C. on a Clariostar plate reader (BMG) kinetically during 22 hours.

### KRAS Aggregation Seeding Assays

Pept-ins were diluted from a 5 mM stock in 6M Urea in PBS to a final concentration of 100 μM in low-binding tubes and incubated during 20 hrs at 37° C. This solution was used either directly in subsequent seeding assays or aliquots were flash-frozen using liquid nitrogen and stored at −80° C. for later seeding assays.

For seeding assays with mature pept-in aggregates, 5 μM of the mature pept-in solution was mixed with 1 mg/ml recombinant mutant KRAS G12V in Hepes buffer containing 200 mM of arginine and glutamine. Seeding was monitored in black 384-well plates (30 μl final volume per well) using ThT as aggregation/amyloid sensor dye at 37° C. on a Clariostar plate reader (BMG).

For seeding assays with pept-in seeds, mature pept-in solutions were diluted 1 in 3 in PBS and sonicated during 5 min using cycles of 5 sec separated by a 3 sec pause. 5 μM of the sonicated pept-in solution was next mixed with 1 mg/ml recombinant mutant KRAS G12V in Hepes buffer containing 200 mM of Arginine and Glutamine. Seeding was monitored in black 384-well plates (30 μl final volume per well) using ThT as amyloid sensor dye at 37° C. on a Clariostar plate reader (BMG).

### In Vitro Translation Assay

In vitro translation assays were performed using the PURExpress® In Vitro Protein Synthesis Kit (New England Biolabs) according to the manufacturer's instructions. Briefly, linear DNA fragments containing T7 promotor and terminator sequences flanking the KRAS coding sequence were generated using PCR and purified using the MinElute PCR Purification Kit (Qiagen). 250 ng of linear DNA was subsequently used for the in vitro translation reaction, which was performed for 2 hours at 37° C. with shaking (1000 rpm). Indicated biotinylated pept-ins were mixed in the translation reactions from a 5 mM stock solution in 6M Urea to a final concentration of 10 μM. Upon completion of the translation reaction, biotinylated pept-ins were captured from the reaction mix using Streptavidin coated beads (Pierce) during 90 min at room temperature. Beads were next washed with TBS containing 0.1% Tween 20 and bound proteins were finally boiled off in 1×SDS loading dye (Bio-Rad) in TBS buffer. Proteins were resolved using Any kD 15-well Mini-PROTEAN gels (Bio-Rad) during SDS-PAGE and probed for KRAS after Western blotting using a mouse monoclonal KRAS-specific antibody (SC-30, Santa Cruz Biotechnology), which was detected with an HRP-coupled anti-mouse secondary antibody using chemiluminescence on a Bio-Rad Chemidoc MP imaging instrument.

### Co-Immunoprecipitation Assays

Cellular co-immunoprecipitation assays were performed using either KRAS wild-type or mutant G12V expressing RASless MEFs (see elsewhere) or human NCI-H441 lung adenocarcinoma tumor cells and N-terminally biotinylated pept-ins. Cells were seeded at a density of 300,000 cells in a clear 6-well plate (Cellstar, Greiner). One day after seeding, cells were treated with indicated pept-ins at a final concentration of 25 μM and incubated for 20 hours. Next, cells were lysed with NP-40 lysis buffer (150 mM NaCl, 50 mM Tris HC1 pH8, 1% IGEPAL(NP40), 1xHalt phosphatase/protease inhibitors (Thermo), 1U/μl Universal Nuclease (Pierce)) and biotinylated pept-ins were captured with streptavidin-coated magnetic beads (Pierce) during 1 hours at room temperature. Beads were washed with NP40 lysis buffer at least 3 times, after which bound proteins were boiled off in 1× SDS loading dye (Bio-Rad) in NP40 lysis buffer. Proteins were resolved using Any kD 15-well Mini-PROTEAN gels (Bio-Rad) during SDS-PAGE and probed for KRAS after Western blotting using a rabbit polyclonal KRAS-specific antibody (12063-1-AP, Proteintech).

### Flow Cytometry

NCI-H441 cells were seeded in a 12-well plate at a density of 175 k cells/well. Next day, cells were treated with vehicle or 12.5 μM of the RAS-targeting pept-ins or the negative control pept-in. After 6. 16 and 24 hours of treatment, cells were washed with PBS and detached using TrypLE Express (Thermo Fisher). Washed cells were next stained using Sytox Blue (Thermo Fisher) and Amytracker Red (Ebba Biotech AB), before analyzing them on a Gallios flow cytometer (Beckman Coulter).

### Cellular Fluorescent Imaging

Fluorescent cellular imaging was performed using HeLa cells that were transduced with lentiviral particles carrying a construct expressing KRAS G12V labeled N-terminally with mCherry. Cells were seeded in a black μclear® Cellstar® F-bottom 96-well plates (Greiner) in 100 μL full growth medium. One day later, cells were treated with indicated FITC-labeled pept-ins in normal growth medium during 20 min after which the pept-in solution was washed off and replaced with normal growth medium again and incubated for an additional 2 hours. Next, cells were fixed, washed and counterstained with the nuclear dye NucBlue™ (containing Hoechst 33342). Images were captured on a Leica confocal microscope.

### In Vivo SW620 Xenograft Model

Female NCr nu/nu mice (8 to 12 weeks) were inoculated with 1×10⁶ SW620 tumor cells in 50% Matrigel subcutaneously in the hind flank. The cell Injection Volume was 0.1 mL/mouse. When tumors reached an average size of 100-150 mm³ a pair match was performed, and treatment started. Group sizes were N=6 for the non-treated group, N=5 for the vehicle groups and N=8 for the pept-in and positive control groups. Tumor growth was monitored by caliper measurement twice per week. Model response was monitored by Irinotecan dosed once per week at 100 mg/kg intraperitoneally for 3 weeks.

### Example 1: Design of RAS-Specific Aggregating Molecules (‘Pept-Ins’)

We used the statistical thermodynamics algorithm TANGO to identify aggregation prone regions (APRs) in the primary amino acid sequence of human RAS family proteins (HRAS, NRAS and KRAS). This analysis showed that all 3 RAS family members have an identical TANGO profile with each of them carrying 5 APRs of at least 5 amino acids in length, of which 2 APRs have a TANGO score of at least 20% (Table 6). The start position (‘Start’) of a given APR as indicated in Table 6, corresponds to the position, in the RAS sequence, of the first N-terminal gatekeeper preceding the respective aggregation prone region per se, whereas elsewhere in this specification the start position of the APR may be given without the N-terminal gatekeeper. Hence, for example, the N-terminal most APR of RAS is stated in Table 6 to start at the M gatekeeper at position 1 of RAS, whereas this APR may be stated to start with T at position 2 elsewhere in this specification. Further in Table 6, ‘N-GKs’ denotes the native gatekeeper residues N-terminally adjacent to the predicted APR in RAS, ‘C-GKs’ denotes the native gatekeeper residues C-terminally adjacent to the predicted APR in RAS, ‘APR seq’ denotes the APR sequence, ‘Score’ means TANGO score in %, and ‘Length’ denotes the APR length (aa) excluding any gatekeepers.

TABLE 6
TANGO analysis of RAS family proteins.

| Protein | Start | N-GKs | APR seq | C-GKs | Score | Length |
|---|---|---|---|---|---|---|
| HRAS | 1 | M | TEYKLV | GVG | 20.2368 | 11 |
| | | | VVGAG | | | |
| | | | SEQ ID | | | |
| | | | NO: 1 | | | |
| HRAS | 17 | GKS | ALTIQLI | QNH | 9.34057 | 7 |
| | | | SEQ ID | | | |
| | | | NO: 75 | | | |
| HRAS | 76 | TGE | GFLCVFAIN | NTK | 68.1289 | 9 |
| | | | SEQ ID | | | |
| | | | NO: 76 | | | |
| HRAS | 110 | DVP | MVLVG | NKC | 3.08482 | 5 |
| | | | SEQ ID | | | |
| | | | NO: 77 | | | |
| HRAS | 154 | VED | AFYTLV | REI | 56.7861 | 6 |
| | | | SEQ ID | | | |
| | | | NO: 78 | | | |
| KRAS | 1 | M | TEYKLV | GVG | 20.5293 | 11 |
| | | | VVGAG | | | |
| | | | SEQ ID | | | |
| | | | NO: 1 | | | |
| KRAS | 17 | GKS | ALTIQLI | QNH | 9.53801 | 7 |
| | | | SEQ ID | | | |
| | | | NO: 75 | | | |
| KRAS | 76 | TGE | GFLCVFAIN | NTK | 68.2723 | 9 |
| | | | SEQ ID | | | |
| | | | NO: 76 | | | |
| KRAS | 110 | DVP | MVLVG | NKC | 3.1616 | 5 |
| | | | SEQ ID | | | |
| | | | NO: 77 | | | |
| KRAS | 154 | VED | AFYTLV | REI | 56.8076 | 6 |
| | | | SEQ ID | | | |
| | | | NO: 78 | | | |
| NRAS | 1 | M | TEYKLV | GVG | 20.1731 | 11 |
| | | | VVGAG | | | |
| | | | SEQ ID | | | |
| | | | NO: 1 | | | |
| NRAS | 17 | GKS | ALTIQLI | QNH | 9.29791 | 7 |
| | | | SEQ ID | | | |
| | | | NO: 75 | | | |
| NRAS | 76 | TGE | GFLCVFAIN | NTK | 67.981 | 9 |
| | | | SEQ ID | | | |
| | | | NO: 76 | | | |
| NRAS | 110 | DVP | MVLVG | NKC | 3.07989 | 5 |
| | | | SEQ ID | | | |
| | | | NO: 77 | | | |
| NRAS | 154 | VED | AFYTLV | REI | 56.4851 | 6 |
| | | | SEQ ID | | | |
| | | | NO: 78 | | | |

Activating mutations in RAS family members are a common and often early event in human cancers and it has been reported that up to one-third of all human tumors carry missense mutations in one of the RAS family members. Greater than 99% of these mutations occur at so-called hotspot mutation sites which are again shared among the RAS family members and are located at codons 12, 13 and 61. Interestingly, codon 12 is located at the C-terminus of an APR, and codon 13 is located immediately adjacent to the C-terminus of an APR, and a missense mutation at one of these positions might therefore alter the aggregation propensity but also the sequence selectivity of the aggregation process (Table 6). To study the former, we analyzed how a set of prevalent mutations (>1% over all KRAS mutant cancers) at codons 12 or 13 alters the TANGO output of the sequence (Table 7). In Table 7, ‘Score’ means TANGO score in %, ‘Length’ denotes the APR length (aa) excluding any gatekeepers, and ‘Frequency’ denotes frequency of the particular G12 or G13 mutation in all KRAS mutant cancers in % based on COSMIC database.

TABLE 7
Impact of common G12 or G13 position
mutations on TANGO analysis.

| Mutation | APR | Score | | |
|---|---|---|---|---|
| at G12 | sequence | (%) | Length | Frequency |
| WT | TEYKLVVVGAG | 20,0763 | 11 | / |
| | SEQ ID NO: 1 | | | |
| G12V | TEYKLVVVGAVGVG | 44,8509 | 14 | 23 |
| | SEQ ID NO: 2 | | | |
| G12D | TEYKLVWGA | 40,8683 | 10 | 34 |
| | SEQ ID NO: 79 | | | |
| G12C | TEYKLVWGACGV | 19,5246 | 13 | 12 |
| | SEQ ID NO: 3 | | | |
| G12A | TEYKLVVVGAAGV | 23,9605 | 13 | 5 |
| | SEQ ID NO: 4 | | | |
| G12S | TEYKLVVVGASG | 18,8902 | 12 | 5 |
| | SEQ ID NO: 5 | | | |
| G12R | TEYKLVWGA | 12,8417 | 10 | 3 |
| | SEQ ID NO: 79 | | | |
| G13D | TEYKLVVVGAG | 34,9655 | 11 | |
| | SEQ ID NO: 1 | | | |
| G13C | TEYKLVVVGAGCV | 18,3813 | 13 | |
| | SEQ ID NO: 81 | | | |
| G13V | TEYKLVVVGAGVVG | 40,8491 | 14 | |
| | SEQ ID NO: 82 | | | |
| GI3R | TEYKLVWGA | 17,3727 | 10 | |
| | SEQ ID NO: 132 | | | |
| G13S | TEYKLVWGAGS | 18,6667 | 12 | |
| | SEQ ID NO: 83 | | | |

The most prevalent mutation at position G12 is G12D. This mutation introduces a negatively charged aspartate which TANGO identifies as a gate-keeper residue, resulting in a slightly shorter APR with an increased TANGO score. However, the impact of the second most prevalent mutation, G12V, on the APR is most profound as it increases both the length as well as the TANGO score of the APR sequence. Other prevalent G12 mutations either shorten or lengthen the APR sequence but do not alter the TANGO score significantly. G13D mutation is also very prevalent and increases the aggregation propensity of the APR without altering its sequence. Hence, it is possible that a pept-in having a stretch corresponding to the wild-type APR may display a preference for downregulating G13D RAS compared to wild-type RAS. The impact of the G13V on the APR is also very profound as it increases both the length as well as the TANGO score of the APR sequence.

Based on these data, we selected the RAS WT and G12V APRs for the design of RAS WT or G12V-selective pept-ins, as embodiments illustrating the feasibility of specifically targeting G12 or G13 mutant human RAS using the interferor technology.

To this end we generated all possible 6 to 10-mers (in the present experiments, the length limit of 10 amino acids was informed by the length capacity of solid phase synthesis) based on the sequence of the APRs using a sliding window approach. Next, the resulting ‘APR windows’ were aligned against the full human proteome to exclude sequences that had exact matches in other proteins than the RAS family members to limit off-target activity of pept-ins containing these sequences. This filtering step resulted in 38 APR windows that were taken further in our pept-in design. For the design we employed the previously devised tandem repeat configuration (see WO2012/123419A1), in which the APR windows are repeated once and are separated by a linker. For the design of the initial screening library, we included variants with both GS and PP linkers. Furthermore, to increase the colloidal stability of these aggregating sequences, gatekeeper residues were introduced flanking each repeat of the APR window in the pept-in. Two positively charged (Arginine (R) and Lysine (K)) and one negatively charged (Aspartate (D)) amino acids were selected and introduced in the screening library. An overview of the resulting pept-in templates with different gate keeper residues and linkers is given in Table 8. The K-APR-KGSK-APR-K template was applied to all APR windows, while the other templates were applied to all APR windows up to 8 amino acids in length.

TABLE 8
Overview of pept-in design
templates for screening.

| Gate-keeper | | |
|---|---|---|
| residue | Linker | Pept-in layout |
| K | GS | K-APR-KGSK-APR-K |
| R | GS | R-APR-RGSR-APR-R |
| K | PP | K-APR-KPPK-APR-K |
| D | PP | D-APR-DPPD-APR-D |
| KK | PP | KK-APR-KPPK-APR-KK |

All pept-ins designed were generated using solid phase synthesis, however, for a few sequences synthesis or purification failed to meet the quality standards (purity>95%) and were therefore excluded from further analysis. Pept-ins for which synthesis and purification was successful were dissolved in 6M Urea to a 5 mM stock and tested for their biological activity.

### Example 2: Activity Screening of RAS-Targeting Pept-Ins

To assess pept-in activity on the viability of RAS-mutant tumor cells, we used adherent NCI-H441 lung adenocarcinoma cells which harbor a G12V mutation in KRAS. To verify that this cell line was indeed dependent on KRAS for its growth, we used SAH-SOS-1A as a positive control. SAH-SOS-1A is a peptidic compound whose design is based on a stabilized helix from son of sevenless 1, the canonical guanine exchange factor for KRAS (Leshchiner et al. Proc Natl Acad Sci U S A. 2015, vol. 112(6), 1761-6). Treatment of NCI-H441 cells with SAH-SOS-1A resulted in a dose-dependent drop in viability with an IC₅₀ of ˜15 μM after 4 days exposure, which was consistent with reported values for other cell lines and established the KRAS-dependence for the NCI-H441 cell line. We also tested Urea tolerance of NCI-H441 cells and found that there was no significant effect on viability up to 60 mM of Urea after 4 days of exposure.

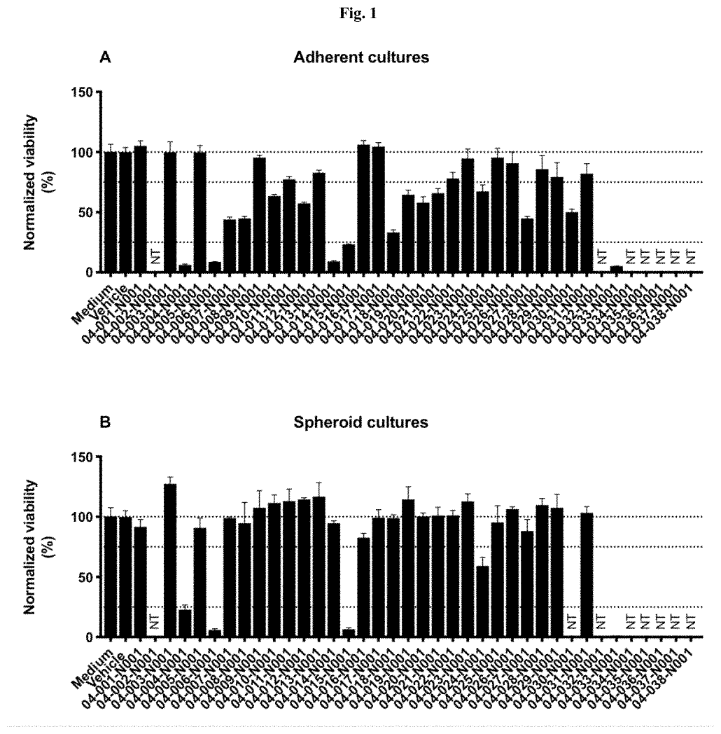

Pept-ins were screened at a single dose of 25 μM (corresponds to final concentration of 30 mM Urea) and viability was measured after 2 and 4 days of exposure using the CellTiter Blue reagent. After 4 days of exposure over half of all K-APR-KGSK-APR-K pept-ins tested (˜52%) induced a reduction of at least 25% in viability as compared to vehicle treated cells (30 mM Urea; FIG. 1A). Hit rates and potencies for the other templates tested were considerably lower. To select potent hits for further characterization, we selected all pept-ins that showed at least 75% decrease in viability after 4 days of exposure. This cut-off resulted in selection of 5 pept-ins, all with the K-APR-KGSK-APR-K template: 04-004-N001, 04-006-N001, 04-014-N001, 04-015-N001 and 04 N001. One of these pept-ins (04-004-N001) harbours an APR window sequence derived from another APR of RAS, that is thus present in both G12 mutant and wild-type RAS, while the other four pept-ins (04-006-N001, 04-014-N001, 04-015-N001 and 04-033-N001) harbour an APR window sequence that is derived from and contains a G12V mutant site. Furthermore, we selected one biologically non-active peptide (04-016-N001) to be used as negative control in later assays.

This pept-in carries a 7-mer APR window that was designed to target RAS G12V but failed to alter viability of the NCI-H441 cells.

The sequences of the aforementioned pept-ins are shown in Table 9.

| | | Normalised |
|---|---|---|
| | | viability |
| | | NCI-H441 |
| | | 4 days of |
| | | exposure |
| | | to |
| Pept-in | | 25 μM |
| code | Sequence | (%) |
| 04-004-N001 | Ac-KLSVFAIKGSK | 6.1 |
| | LSVFAIK-NH2 | |
| 04-006-N001 | Ac-KVVVGAVKGSK | 8.7 |
| | VVVGAVK-NH2 | |
| 04-014-N001 | Ac-KLVVVGAVKGS | 9.1 |
| | KLVVVGAVK-NH2 | |
| 04-015-N001 | AC-KVVVGAVGKGS | 23.3 |
| | KVVVGAVGK-NH2 | |
| 04-033-N001 | AC-KVVVGAVGVGK | 5.2 |
| | GSKVVVGAVGVG | |
| | K-NH2 | |

The amino acid sequence of pept-in 04-004-N001 as shown in Table 9 is assigned SEQ ID NO: 80, while the amino acid sequences of pept-ins 04-006-N001, 04-014-N001, 04-015-N001 and 04-033-N001 are represented as SEQ ID NO: 15, SEQ ID NO: 16, SEQ ID NO: 17, and SEQ ID NO: 18, respectively, as also set forth elsewhere in this specification. ‘Ac’ in Table 9 denotes N-terminus acetylation, and ‘NH2’ in Table 9 denotes C-terminus amidation.

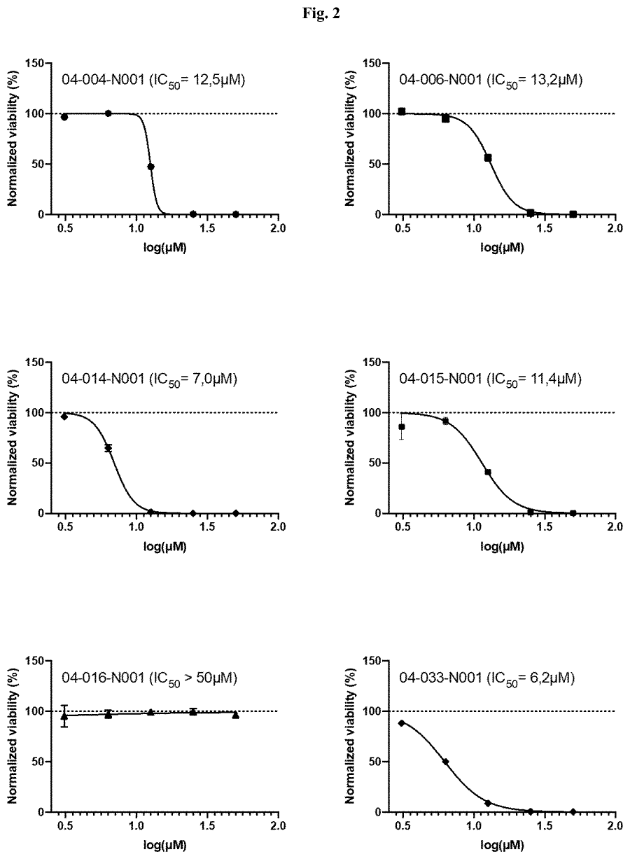

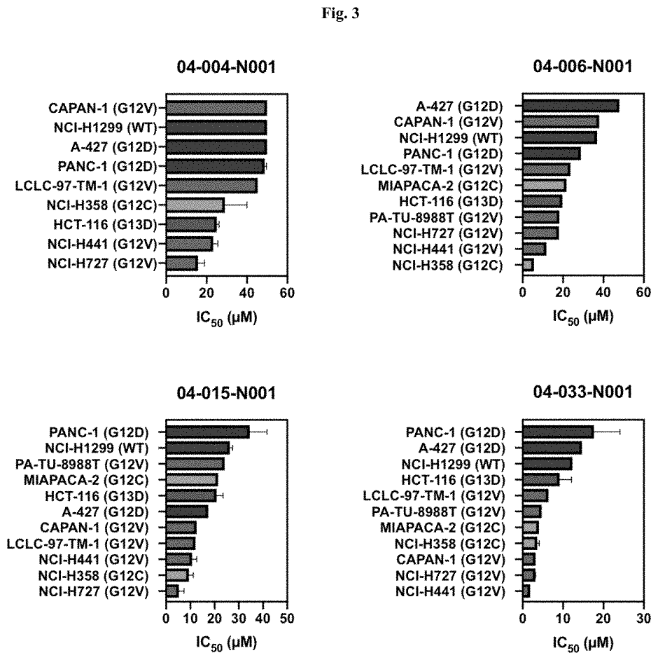

These 6 pept-ins were resynthesized and purified to test their potency in reducing viability of adherently growing ('2D viability assay') NCI-H441 cells in dose-response. To this end, pept-ins were tested in a five-point dose-response using a one-in-two dilution series starting from 50 μM as highest dose on adherently growing NCI-H441 cells. Viability was assessed three days after of exposure to the test compounds using the CellTiter Glo viability assay. This analysis showed that the 5 active compounds all showed IC5os around 10 μM (FIG. 2).

As previous reports have shown that adherent growth of KRAS mutant cells lines might attenuate their sensitivity to KRAS inhibition or knockdown (Fujita-Sato et al. Cancer Res. 2015, vol. 75, 2851-62; Patricelli et al. Cancer Discov. 2016, vol. 6, 316-29; Vartanian et al. J Biol Chem. 2013, vol. 288, 2403-13), we complemented the screen on adherently growing NCI-H441 cells with a screen on suspension spheroid cultures of the same cell line. To this end, NCI-H441 cells were seeded in ultra-low adherent round bottom plates allowing formation of spheroids. As for the adherent screen, we adopted a single-dose approach using 25 μM of each test pept-in. Viability of the spheroid cultures was determined after 5 days of exposure using the CellTiter Glo 3D reagent from Promega. Hit rates using this approach were considerably lower as compared to the adherent screen described above (FIG. 1B). Indeed, while in the adherent setting over half of all K-APR-KGSK-APR-K pept-ins tested induced a reduction of at least 25% in viability as compared to vehicle treated cells, in the spheroid setting only 17% of this set of pept-ins reduced viability with more than 25%. Furthermore, hit rates and potencies for the other templates tested were also lower. Of note, applying the same selection criterion for potent hits here as for the adherent screen, i.e. selecting pept-ins that showed at least 75% decrease in viability after 5 days of exposure, resulted in the selection of same pept-ins as in the adherent screen, with the exception of 04-014-N001, which did not display activity in the spheroid setting.

The suspension spheroid approach was used next to assess efficacy of the four active pept-ins on a larger set of KRAS mutant and wild-type tumor cells lines. Waterfall plots for each pept-in showing the median IC₅₀ for these cell lines are shown in FIG. 3.

The suspension spheroid approach was used next to assess efficacy of various versions of the 04-004, 004-006, 04-015 and 04-033 pept-ins containing alternative gatekeeper and/or linker parts, in NCI-H441 lung adenocarcinoma cells. IC50 on cell viability were determined using the CellTiter Glo 3D assay (Promega) after 5 days of exposure to a dose-response of each pept-in. The pept-ins and the respective IC50 values are listed in Table 12 below (‘Ac’ denotes N-terminus acetylation; ‘NH2 ’ denotes C-terminus amidation; ‘[Dap]’ denotes diaminopimelic acid; ‘[Cit]’ denotes citrulline; L-amino acids are represented using capital letter coding; D-amino acids are represented by small letter coding):

TABLE 12
IC50 on cell viability for various pept-ins as disclosed herein

| | APR/ | Full sequence/ | IC50 |
|---|---|---|---|
| Pept-ins | SEQ ID NO | SEQ ID NO | (μM) |
| 04-004-N021 | LSVFAI | Ac-kLSVFAIKGSKLSVFAIk-NH2 | 34.6 |
| | 155 | 156 | |
| 04-006-N021 | VVVGAV | Ac-kVVVGAVKGSKVVVGAVk-NH2 | 49.9 |
| | 10 | 157 | |
| 04-015-N009 | VVVGAVG | Ac-kVVVGAVGKGSKVVVGAVGk-NH2 | 16.4 |
| | 12 | 158 | |
| 04-033-N021 | VVVGAVGVG | Ac-kVVVGAVGVGKGSKVVVGAVGVGk-NH2 | 8.4 |
| | 13 | 159 | |
| 04-004-N022 | LSVFAI | Ac-[Dap]LSVFAIKGSKLSVFAI[Dap]-NH2 | 19.9 |
| | 155 | 160 | |
| 04-006-N022 | VVVGAV | Ac-[Dap]VVVGAVKGSKVVVGAV[Dap]-NH2 | 19.1 |
| | 10 | 161 | |
| 04-015-N012 | VVVGAVG | Ac-[Dap]VVVGAVGKGSKVVVGAVG[Dap]- | 25.6 |
| | 12 | NH2 | |
| | | 162 | |
| 04-033-N022 | VVVGAVGVG | Ac- | 5.0 |
| | 13 | [Dap]VVVGAVGVGKGSKVVVGAVGVG[Dap]- | |
| | | NH2 | |
| | | 163 | |
| 04-006-N074 | VVVGAV | Ac-[Cit]VVVGAVKGSKVVVGAVK-NH2 | 25.3 |
| | 10 | 164 | |
| 04-006-N075 | VVVGAV | Ac-KVVVGAV[Cit]GSKVVVGAVK | 6.5 |
| | 10 | 165 | |
| 04-006-N044 | VVVGAV | AC-AVVVGAVKGSKVVVGAVK-NH2 | 5.3 |
| | 10 | 166 | |
| 04-006-N050 | VVVGAV | AC-KVVVGAVAGSKVVVGAVK-NH2 | 5.7 |
| | 10 | 167 | |
| 04-006-N053 | VVVGAV | Ac-KWVGAVKGSAVVVGAVK-NH2 | 21.6 |
| | 10 | 168 | |
| 04-006-N059 | VVVGAV | Ac-KVVVGAVKGSKVVVGAVA-NH2 | 15.7 |
| | 10 | 169 | |
| 04-006-N060 | VVVGAV | Ac-AVVVGAVKGSAVVVGAVK-NH2 | 31.7 |
| | 10 | 170 | |
| 04-006-N066 | VVVGAV | Ac-KVVVGAVAGSKVVVGAVA-NH2 | 25.3 |
| | 10 | 171 | |
| 04-006-N082 | VVVGAV | Ac-AVVVGAVAGSKVVVGAVK-NH2 | 18.2 |
| | 10 | 172 | |
| 04-006-N051 | VVVGAV | AC-KVVVGAVKASKVVVGAVK-NH2 | 10.4 |
| | 10 | 173 | |
| 04-006-N052 | VVVGAV | AC-KVVVGAVKGAKVVVGAVK-NH2 | 30.6 |
| | 10 | 174 | |
| 04-015-N063 | VVVGAVG | AC-KVVVGAVGKGFKVVVGAVGK-NH2 | 38.7 |
| | 12 | 175 | |
| 04-015-N064 | VVVGAVG | AC-KVVVGAVGKFFKVVVGAVGK-NH2 | 48.9 |
| | 12 | 176 | |
| 04-004-N016 | LSVFAI | Ac-KLSVFAIKKLSVFAIK-NH2 | 45.9 |
| | 155 | 177 | |
| 04-033-N007 | VVVGAVGVG | AC-KVVVGAVGVGKKVVVGAVGVGK-NH2 | 13.1 |
| | 13 | 178 | |
| 04-004-N030 | Isvfai | Ac-klsvfaikGsklsvfaik-NH2 | 21.4 |
| | | 179 | |
| 04-006-N030 | vVVGav | Ac-kwvGavkGskvvvGavk-NH2 | 15.7 |
| | | 180 | |
| 04-033-N030 | vvvGavGvG | Ac-kwvGavGvGkGskvvvGavGvGk-NH2 | 5.5 |
| | | 181 | |

Table 12 shows that persuasive IC50 values on cell viability have been demonstrated by molecules which exemplify various embodiments of the pept-ins as disclosed herein, such as, peptin-ins containing one or more D-lysine (‘k’), diaminopimelic acid (‘[Dap]’), citrulline (‘[Cit]’), or L-alanine (‘A’) within one or more of their gatekeeper stretches; one or more L-alanine (‘A’) or L-phenylalanine (‘F’), or one or more D-serine (‘s’) within their linker moiety or even not comprising any linker moiety; and/or composed entirely of D-amino acids and glycine. These pept-ins demonstrate the structural flexibility of the present approach focused on targeting the aggregation-prone stretches within proteins.

### Example 3: RAS-Targeting Pept-Ins are Aggregation-Prone and Seed Aggregation of RAS Through Direct Interaction In Vitro

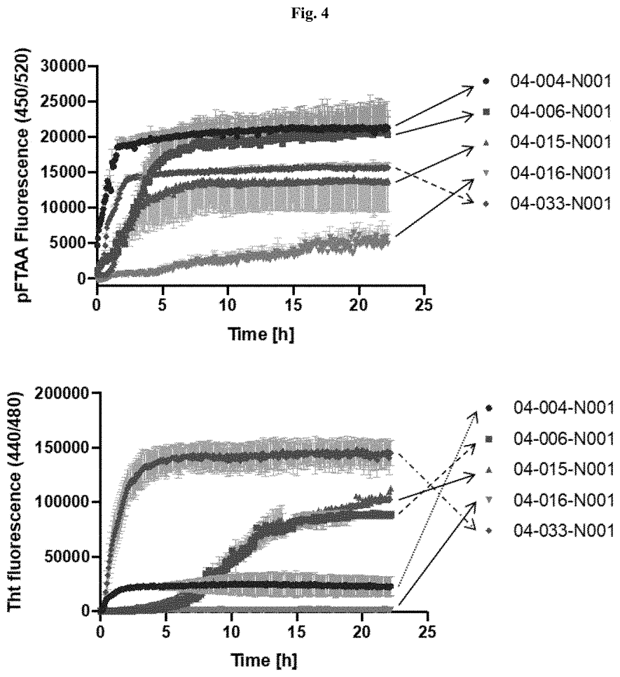

To study the aggregation behaviour of the RAS-targeting pept-ins, we performed kinetic tinctorial assays using the amyloid aggregate sensor dyes Thioflavin T (ThT) and pentameric formyl thiophene acetic acid (p-FTAA). All four representative biologically active pept-ins showed clear amyloid-aggregation kinetics with both dyes, while the inactive control showed no significant ThT signal and only a slight increase in p-FTAA signal over time (FIG. 4).

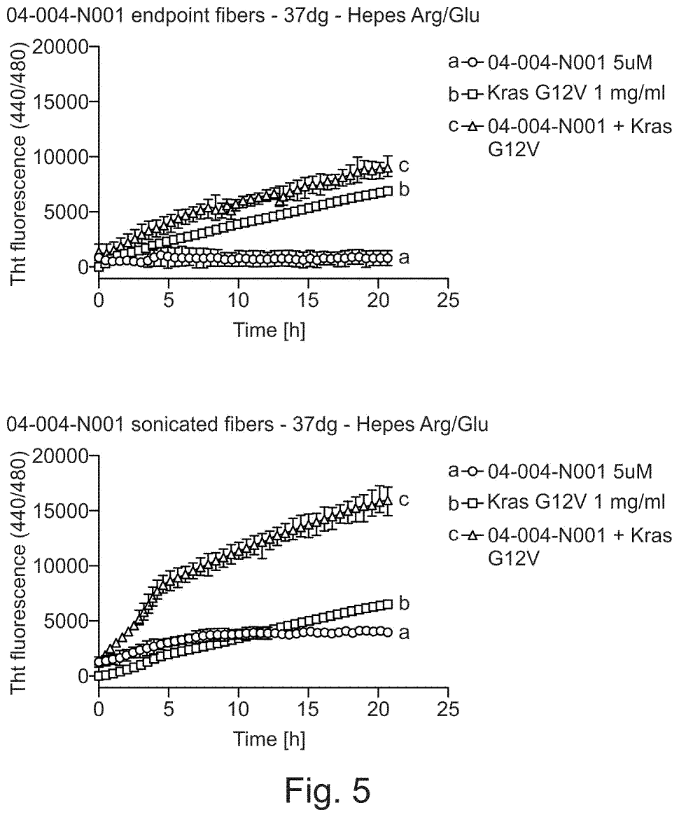

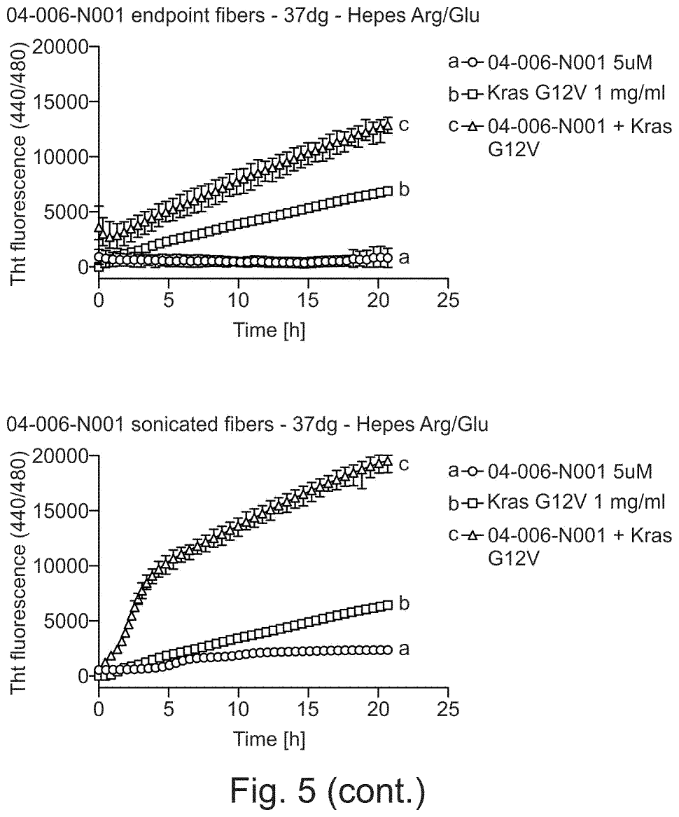

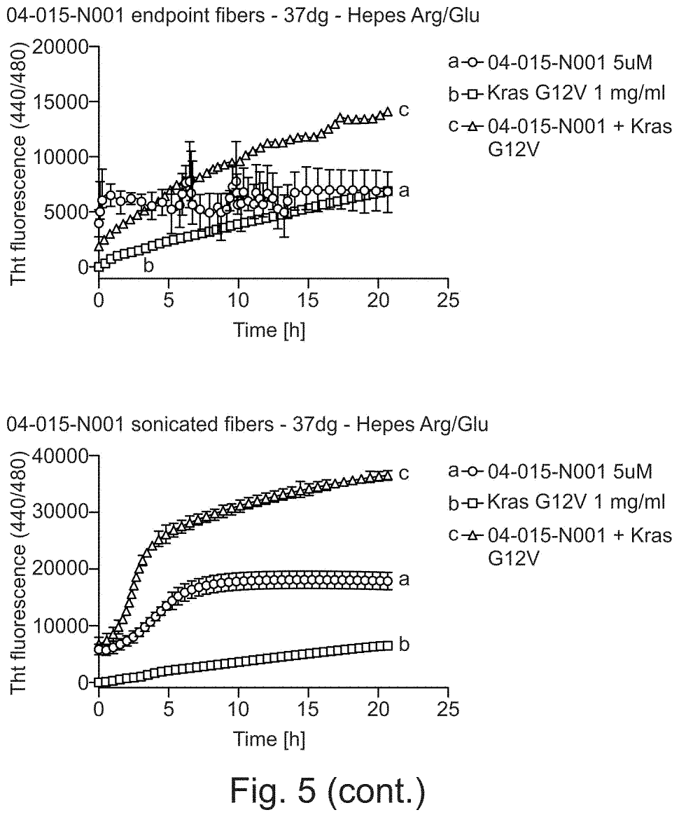

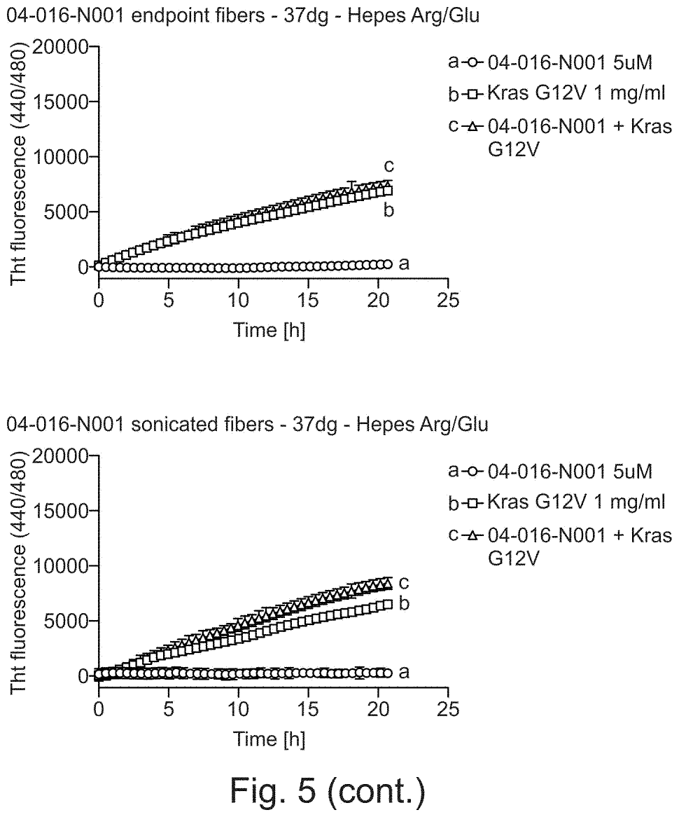

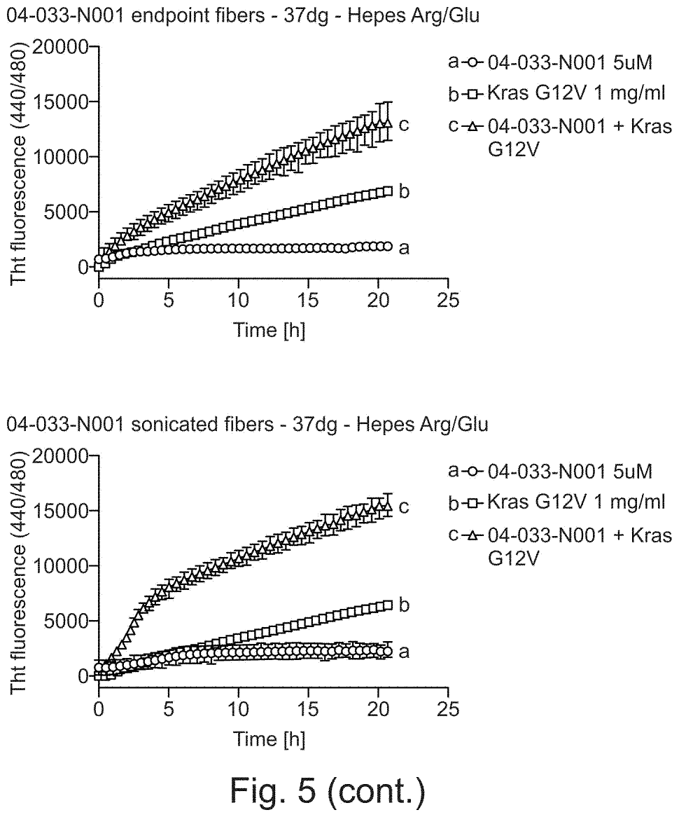

To show that the illustrative biologically active pept-ins are indeed able to target and seed the aggregation of their target protein, KRAS G12V, we performed seeding experiments with end-stage aggregates or sonicated seeds of the different KRAS-targeting pept-ins. To this end, pept-ins were allowed to aggregate in the same timeframe as for the tinctorial kinetic assays. End-stage samples were then mixed with recombinantly produced KRAS G12V and aggregation was monitored kinetically using ThT. This approach revealed only minor seeding capacity of these end-stage pept-in aggregates on KRAS G12V. However, upon disruption of the mature aggregates through sonication, potent seeds are formed which efficiently induce aggregation of KRAS G12V (FIG. 5).

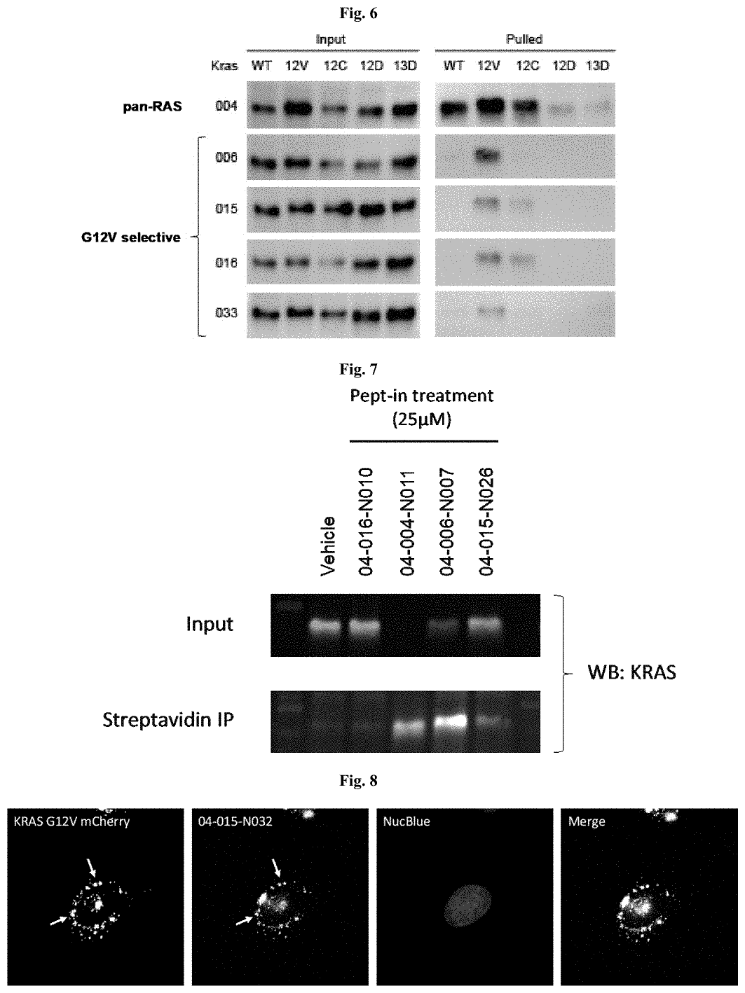

To show that the RAS-targeting pept-ins interact directly with the RAS protein we setup an in vitro translation assay. Indeed, as the available structural data show that the RAS APRs may not be exposed in the native fold, we hypothesize that initial interaction of pept-ins with their target occurs at the ribosome while the protein is being translated and briefly exposes these APRs. To mimic this in vitro, we devised an in vitro translation setup producing either wild-type or mutant (G12V, G12C, G12D or G13D) KRAS in the presence of biotinylated RAS-targeting pept-ins. This allowed us to perform a streptavidin pull-down to capture the biotinylated pept-ins from the translation reaction and perform SDS-PAGE and Western blotting to probe the pulled-down fraction for the presence of KRAS. The biotinylated version of pept-in 04-004-N001, i.e. 04-004-N011, which harbours an APR window sequence derived from a wild-type APR, is predicted to target all RAS proteins independently from their mutation status. While efficient pull-down with 04-004-N011 was indeed observed for KRAS wild-type, G12V and G12C, binding to the G12D and G13D mutants appeared to be less efficient. Using the biotinylated versions of the biologically active pept-ins harbouring an APR window containing the G12V mutant site (04-006-N007, 04-015-N026 and 04-033-N003), however, notable pull-down was only observed for the G12V mutant KRAS and, in the case of 04-015-N026, for the G12C mutant KRAS (FIG. 6).

Together, these data show that these illustrative RAS-targeting pept-ins are able to directly interact with and seed the aggregation of RAS proteins containing an exact match for the APR windows present in the pept-ins.

### Example 4: Mutant-Selective Cellular Efficacy in the RASless MEF System

RAS mutant-selectivity on cellular efficacy was assessed using the isogenic RASless mouse embryonic fibroblast (MEF) panel. These MEFs are derived from NRAS- and HRAS-null mice in which the KRAS gene has been floxed as well (removal by ER-Cre). Proliferation is dependent on the expression of either the endogenous KRAS gene or—if it has been removed through tamoxifen treatment—on an expressed transgene. The panel assessed included the common clinical KRAS variants expressed as transgene (WT, G12V and G12C) and an additional cell line dependent on the expression of BRAF V600E for proliferation. The latter should be refractory to KRAS targeting agents as they do not express any of the RAS isoforms and proliferation of these cells is exclusively dependent on mutant BRAF, which is downstream of RAS.

Efficacy of RAS-targeting pept-ins on MEFs growing as spheroids was assessed after 5 days of exposure. As the targeting moiety of 04-004-N001 is an APR-window derived from a wild-type RAS sequence, it is predicted to target all RAS-dependent growth, independent from mutation status. Surprisingly, however, notable increased efficacy of 04-004-N001 was observed for the MEFs expressing KRAS G12V as compared to the KRAS WT and G12C expressing MEFs, which responded similarly as the BRAF V600E expressing RASless MEFs.

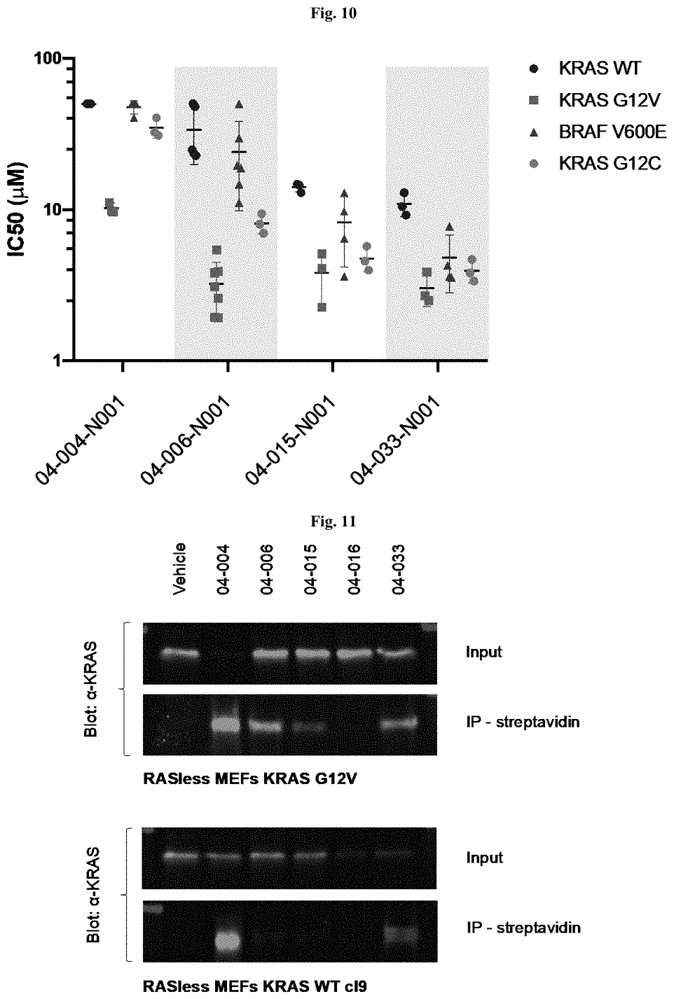

For the G12V-targeting RAS pept-ins the highest efficacy was observed when assessing the G12V-expressing RASless MEFs, indicating that mutant-selective binding at least in part drives, and may be a major contributor to, the selectivity for mutant RAS displayed by these pept-ins. The data is shown in FIG. 10.

### Example 5: RAS-Targeting Pept-Ins Interact with KRAS

To assess whether the RAS-targeting pept-ins are also able to interact with the (mutant) KRAS protein in cells, we setup a co-immunoprecipitation assay.

First, we used the KRAS wild-type and mutant G12V-expressing RASless MEFs to assess whether (i) the RAS-targeting pept-ins bind the KRAS protein in a cellular environment and (ii) whether any binding shows similar G12V mutant-selectivity as observed in the in vitro translation assay described in Example 4. To this end, relevant MEF cells were treated with 25 μM biotinylated pept-ins overnight (16 hours). Next, cells were lysed, and pept-ins were immunoprecipitated from the lysates using streptavidin-coated beads. Precipitated fractions were next resolved using SDS PAGE and probed for the presence of KRAS protein using Western blot. Results show that the 04-004-derived biotinylated pept-in appeared to precipitate both wild-type and mutant G12V KRAS well after 16-hour treatment of the respective RASless MEF cells. Treatment and precipitation with the biotinylated versions of the G12V-selective pept-ins, however, showed preferential binding to the G12V mutant KRAS protein (FIG. 11).

Next, we assessed whether the RAS-targeting pept-ins showed binding to KRAS after exposure to human tumor cells. To this end, the KRAS G12V mutant NCI-H441 lung adenocarcinoma cells were treated with 25 μM biotinylated pept-ins overnight (16 hrs). Next, cells were lysed, and pept-ins were immunoprecipitated from the lysates using streptavidin-coated beads. Precipitated fractions were next resolved using SDS PAGE and probed for the presence of KRAS protein using Western blot. While this approach yielded no detectable KRAS protein in the precipitated fractions from vehicle or negative control peptide-treated conditions, KRAS protein was readily detected in the precipitated fractions from NCI-H441 cells treated with the biologically active pept-ins (FIG. 7).

To complement the co-immunoprecipitation approach, we also used a cellular imaging approach to show target engagement. To this end, we generated a HeLa cell line overexpressing mCherry-tagged KRAS G12V and FITC-labelled versions of the RAS-targeting pept-ins. Treatment of these HeLa cells showed that the FITC-labelled versions of all biologically active RAS-targeting pept-ins are readily taken up by cells, while uptake of the FITC-labelled version of the negative control pept-in 04-016-N001 was not detectable, hence explaining the lack of biological activity. Furthermore, this analysis showed that rapidly after entering the cells, the RAS-targeting FITC-labelled version of pept-in 04-015-N001 (04-015-N032) associates with mCherry-labelled KRAS as revealed by the occurrence of inclusion-like perinuclear structures that are positive for both FITC as well as mCherry 75 min after treatment with the FITC-labeled pept-in (FIG. 8).

### Example 6: RAS-Targeting Pept-Ins Drive Its Aggregation and Degradation in Cells

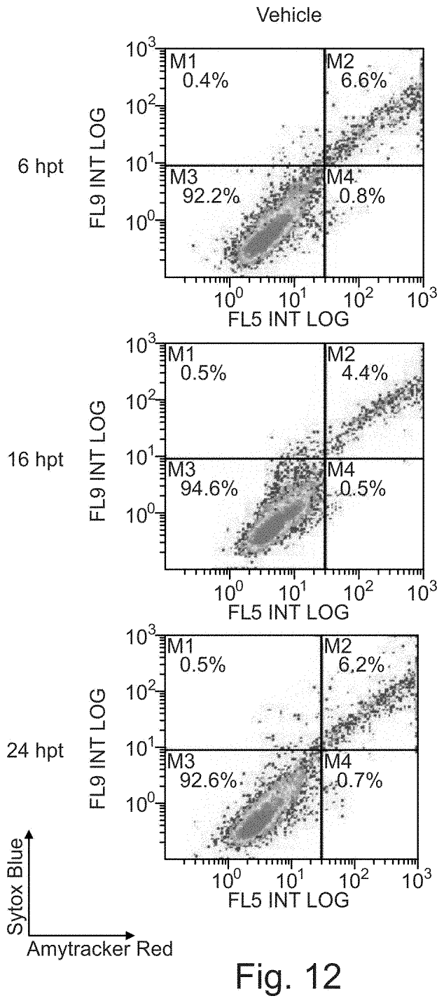

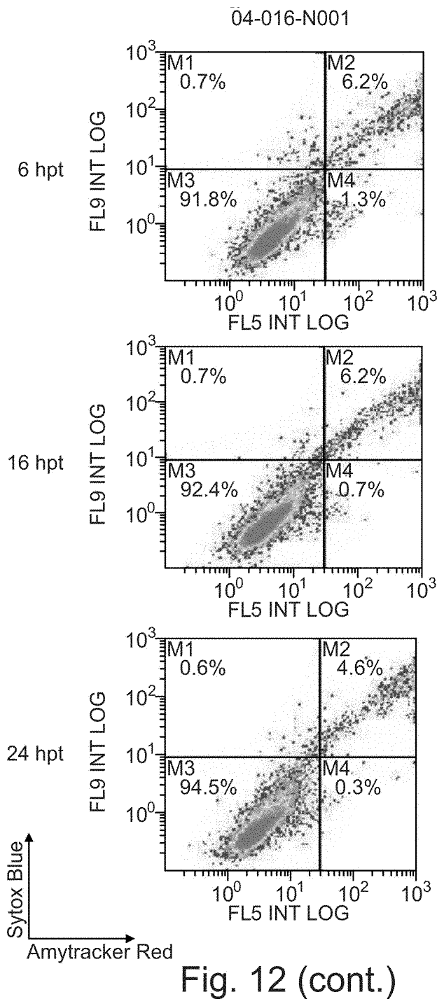

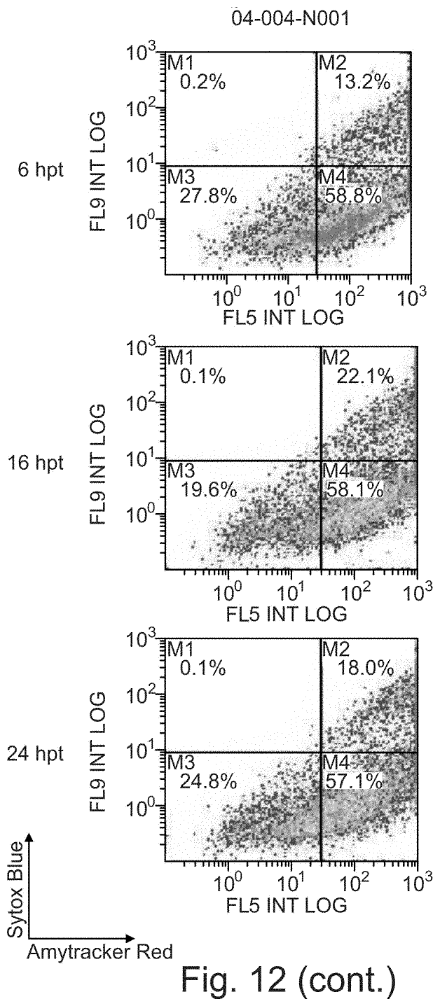

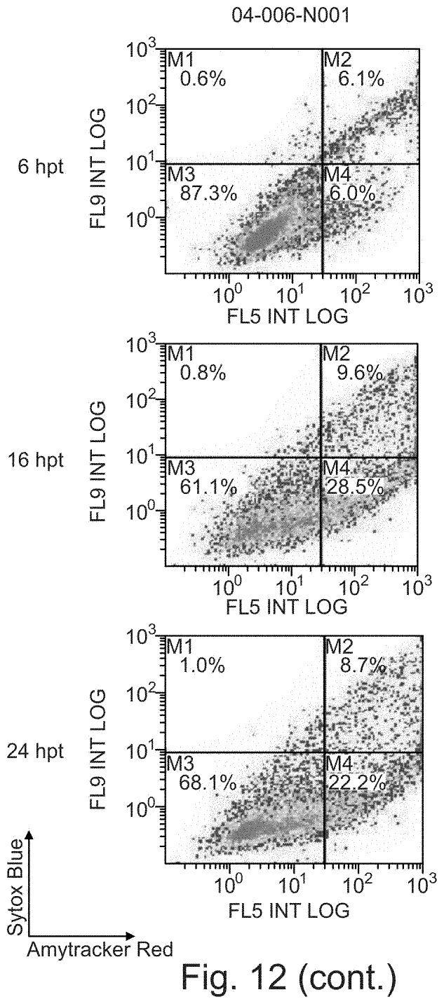

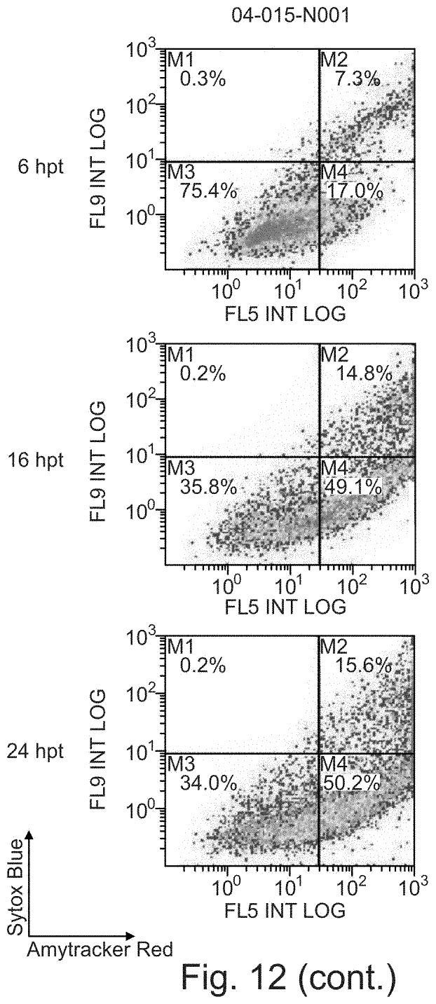

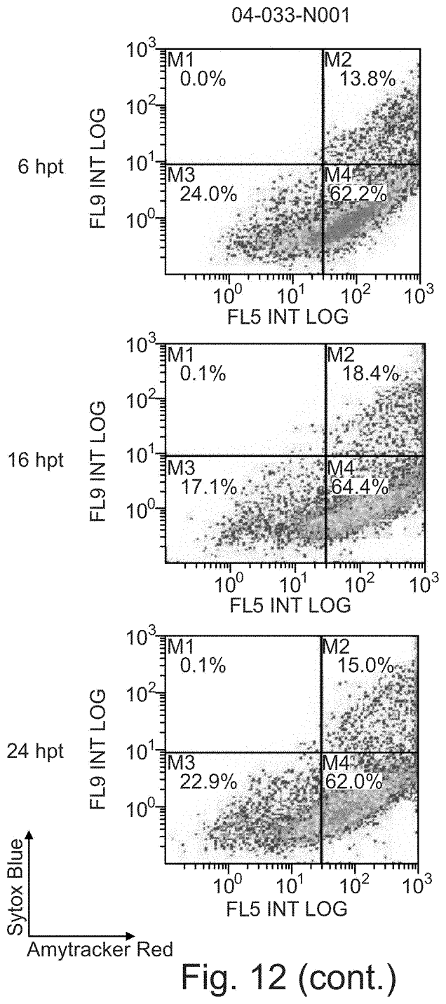

To assess whether treatment of tumor cells with the RAS-targeting pept-ins induces protein aggregation prior to inducing cell death, a flow cytometry assay was devised to monitor cell death in parallel with protein aggregation. To this end, NCI-H441 cells were treated for either 6, 16 or 24 hrs with a near-IC₅₀ dose of the RAS-targeting pept-ins (12.5 μM ) or control conditions (vehicle and negative control pept-in). After treatment, cells were collected and stained for cell death using the Sytox™ Blue dye and for the presence of (amyloid-like) protein aggregates using the Amytracker™ Red dye. This analysis showed that for vehicle and control pept-in treated cells no significant cell death or protein aggregation was observed during the course of the experiment. However, upon treatment with the RAS-targeting pept-ins, protein aggregation was readily detected and appeared to progress over time. Furthermore, this increase in protein aggregation was paralleled with a slow increase in cell death, which appeared to be secondary to the occurrence of protein aggregation (FIG. 12).

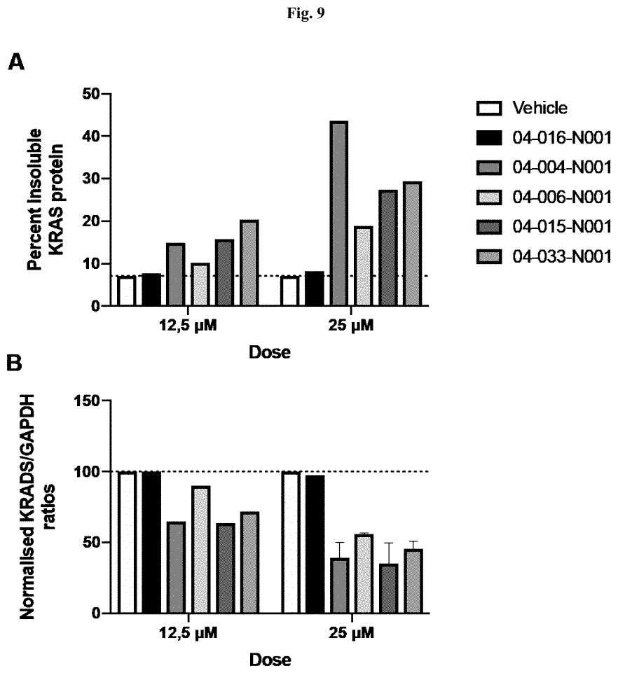

As the flow cytometry assay described above does not offer granularity as to whether the protein aggregation observed was affecting KRAS, we set out to assess KRAS aggregation in a solubility fractionation assay. To this end, NCI-H441 cells were treated with a near IC50 dose (12.5 μM) and a near 2XIC50 dose (25 μM) for 24 hrs. After treatment cells were lysed using a mild, non-denaturing buffer and proteins not soluble in this buffer were pelleted by centrifugation. Insoluble proteins were next solubilized using a strong chaotropic agent, i.e. 6M Urea. Using this approach, amyloid(-like) aggregates are expected to end up in the insoluble fraction. Both the soluble and insoluble fractions were resolved using SDS PAGE and probed for KRAS and GAPDH in a subsequent Western blot. This analysis showed that all biologically active RAS-targeting peptides dose-dependently increased the percentage of KRAS in the insoluble fraction while the percentage of insoluble KRAS was comparable between vehicle and negative control peptide treated samples, indicating that pept-in treatment indeed results in aggregation of the KRAS target protein. To complement these findings, we also quantified the total KRAS levels in these samples (i.e. sum of KRAS levels in the soluble and insoluble fraction for each treatment). Analysis of these data showed that total KRAS levels were also dose-dependently reduced in the samples treated with the biologically active RAS-targeting pept-ins (FIG. 9).

Together, these data show that also in cells the biologically active RAS-targeting pept-ins are able to interact with their intended target protein KRAS and induce its aggregation, as evidenced by the increase in insoluble KRAS protein upon treatment with the pept-ins. Furthermore, presumably, but without implying any limitation to a specific mechanism, as a secondary consequence to aggregation, total KRAS levels are also reduced after treatment with the active pept-ins.

### Example 7: RAS-Targeting Pept-Ins Reduce Tumor Growth in a Xenograft Model of KRAS G12V Mutant Cancer

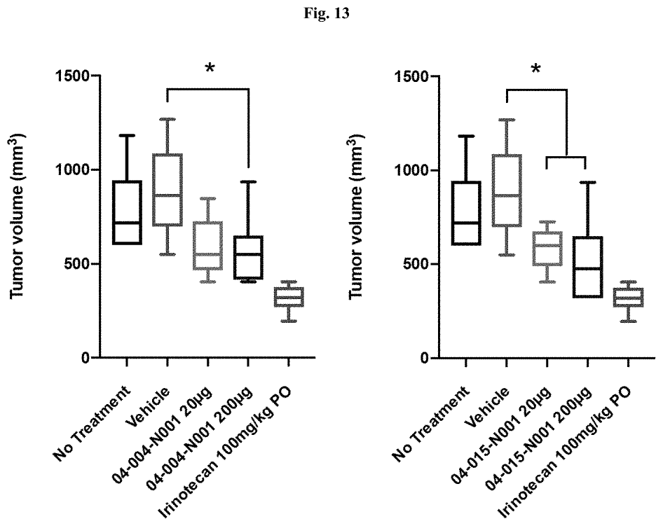

To assess whether the RAS-targeting pept-ins are able to attenuate growth of KRAS G12V-driven tumors in vivo, a subcutaneous xenograft model of human KRAS G12V colorectal cancer (SW620) was used. Once the tumors reached 100-150mm³ in size, pept-ins were administered directly into the tumor mass by intratumoral injection three times per week during two weeks at two different doses (20 μg and 200μg). From the set of pept-ins carrying a G12V-selective RAS APR window sequence (04-006-, 04-015-, and 04-033-N001), 04-015-N001 induced the strongest reduction in tumor growth, as evidenced by a significant reduction in average tumor volume for both the 20 μg and 200 μg dosing groups at day 22 after treatment started. Furthermore, a similar reduction in tumor growth was observed for 04-004-N001, carrying a wild-type RAS APR window sequence, which, however, was only significant for the 200 μg dosing group (FIG. 13).

1. A non-naturally occurring molecule configured to form an intermolecular beta-sheet with a human RAS protein mutated at position 12 or 13 and substantially not with wild-type human RAS protein.

2. The molecule according to claim 1, wherein the mutant human RAS protein is a G12V, G12C, G12A, or G12S mutant human RAS protein, preferably a G12V mutant human RAS protein, or is a G13V, G13C, or G13S mutant human RAS protein, preferably a G13V mutant human RAS protein.

3. The molecule according to claim 1, wherein the RAS protein is KRAS, NRAS or HRAS protein, preferably KRAS protein.

4. The molecule according to claim 1, wherein the intermolecular beta-sheet involves the amino acid at position 12 or 13 of the mutant human RAS protein.

5. The molecule according to claim 4, wherein the intermolecular beta-sheet involves:
a) a portion of or the whole of the amino acid sequence TEYKLVVVGAVGVG (SEQ ID NO: 2) in the G12V mutant human RAS protein; or
b) a portion of or the whole of the amino acid sequence TEYKLVVVGACGVG (SEQ ID NO: 6) or preferably TEYKLVVVGACGV (SEQ ID NO: 3) in the G12C mutant human RAS protein; or
c) a portion of or the whole of the amino acid sequence TEYKLVVVGAAGVG (SEQ ID NO: 7) or preferably TEYKLVVVGAAGV (SEQ ID NO: 4) in the G12A mutant human RAS protein; or
d) a portion of or the whole of the amino acid sequence TEYKLVWGASGVG (SEQ ID NO: 8) or preferably TEYKLVVVGASGV (SEQ ID NO: 9) or more preferably TEYKLVVVGASG (SEQ ID NO: 5) in the G12S mutant human RAS protein; or
e) a portion of or the whole of the amino acid sequence TEYKLVVVGAGVVG (SEQ ID NO: 82) in the G13V mutant human RAS protein; or
f) a portion of or the whole of the amino acid sequence TEYKLVVVGAGCVG (SEQ ID NO: 84) or preferably TEYKLVVVGAGCV (SEQ ID NO: 81) in the G13C mutant human RAS protein; or
g) a portion of or the whole of the amino acid sequence TEYKLVVVGAGSVG (SEQ ID NO: 85) or preferably TEYKLVVVGAGSV (SEQ ID NO: 86) or more preferably TEYKLVVVGAGS (SEQ ID NO: 83) in the G13S mutant human RAS protein.

6. The molecule according to claim 1, wherein the molecule is able to decrease the solubility or to induce the aggregation or inclusion body formation of the human RAS protein mutated at position 12 or 13.

7. The molecule according to claim 1, wherein the molecule comprises an amino acid stretch which participates in the intermolecular beta-sheet.

8. The molecule according to claim 7, wherein the amino acid stretch comprises at least 6 contiguous amino acids of the amino acid sequence:
a)
(SEQ ID NO: 2)
TEYKLVVVGAVGVG;
or
b)
(SEQ ID NO: 6)
TEYKLVVVGACGVG
or preferably
(SEQ ID NO: 3)
TEYKLVVVGACGV;
or
c)
(SEQ ID NO: 7)
TEYKLVVVGAAGVG
or preferably
(SEQ ID NO: 4)
TEYKLVVVGAAGV;
or
d)
(SEQ ID NO: 8)
TEYKLVVVGASGVG
or preferably
(SEQ ID NO: 9)
TEYKLVVVGASGV
or more preferably
(SEQ ID NO: 5)
TEYKLVVVGASG;
or
e)
(SEQ ID NO: 82)
TEYKLVVVGAGVVG;
or
f)
(SEQ ID NO: 84)
TEYKLVVVGAGCVG
or preferably
(SEQ ID NO: 81)
TEYKLVVVGAGCV;
or
g)
(SEQ ID NO: 85)
TEYKLVVVGAGSVG
or preferably
(SEQ ID NO: 86)
TEYKLVVVGAGSV
or more preferably
(SEQ ID NO: 83)
TEYKLVVVGAGS;
including the amino acid at position 11 of the respective sequences a) to d), or the amino acid at position 12 of the respective sequences e) to g).

9. The molecule according to claim 7, wherein the molecule comprises the amino acid stretch VWGAV (SEQ ID NO: 10), LVVGAV (SEQ ID NO: 11), VVVGAVG (SEQ ID NO: 12), VVVGAVGVG (SEQ ID NO: 13), VVVGAGV (SEQ ID NO: 99), LVVVGAGV (SEQ ID NO: 100), VVVGAGVV (SEQ ID NO: 101) or VVVGAGVVG (SEQ ID NO: 102).

10. The molecule according to claim 7, wherein the amino acid stretch comprises one or more D-amino acids and/or analogues of one or more of its amino acids.

11. The molecule according to claim 7, wherein the molecule comprises two or more, preferably two, said amino acid stretches, which are identical or different.

12. The molecule according to claim 7, wherein the amino acid stretch or stretches are each independently flanked, on each end independently, by one or more amino acids that display low beta-sheet forming potential or a propensity to disrupt beta-sheets.

13. The molecule according to claim 7, wherein the molecule comprises, consists essentially of or consists of the structure:
a) NGK1-P1-CGK1,
b) NGK1-P1-CGK1-Z1-NGK2-P2-CGK2,
c) NGK1-P1-CGK1-Z1-NGK2-P2-CGK2-Z2-NGK3-P3-CGK3, or
d) NGK1-P1-CGK1-Z1-NGK2-P2-CGK2-Z2-NGK3-P3-CGK3-Z3-NGK4-P4-CGK4,
wherein:
P1 to P4 each independently denote an amino acid stretch that participates in the intermolecular beta-sheet,
NGK1 to NGK4 and CGK1 to CGK4 each independently denote 1 to 4 contiguous amino acids that display low beta-sheet forming potential or a propensity to disrupt beta-sheets, such as 1 to 4 contiguous amino acids selected from the group consisting of R, K, D, E, P, N, S, H, G, Q, and A, D-isomers and/or analogues thereof, and combinations thereof, preferably 1 to 4 contiguous amino acids selected from the group consisting of R, K, D, E, P, N, S, H, G, and Q, D-isomers and/or analogues thereof, and combinations thereof, more preferably 1 to 4 contiguous amino acids selected from the group consisting of R, K, D, E, and P, D-isomers and/or analogues thereof, and combinations thereof, and
Z1 to Z3 each independently denote a direct bond or preferably a linker.

14. The molecule according to claim 13, wherein:
NGK1 to NGK4 and CGK1 to CGK4 is each independently 1 to 2 contiguous amino acids selected from the group consisting of R, K, A, and D, D-isomers and/or analogues thereof, and combinations thereof, preferably NGK1 to NGK4 and CGK1 to CGK4 is each independently 1 to 2 contiguous amino acids selected from the group consisting of R, K, and D, D-isomers and/or analogues thereof, and combinations thereof, such as wherein NGK1 to NGK4 and CGK1 to CGK4 is each independently K, R, D, A or KK, preferably each independently K, R, D or KK; and/or
each linker is independently selected from a stretch of between 1 and 10 units, preferably between 1 and 5 units, wherein a unit is each independently an amino acid or poly(ethylene glycol) (PEG), such as wherein each linker is independently GS, PP or GSGS (SEQ ID NO: 14), preferably GS, or D-isomers and/or analogues thereof.

15. The molecule according to claim 13, wherein the molecules comprises, consists essentially of or consists of a peptide of the amino acid sequence:
a)
(SEQ ID NO: 15)
KVVVGAVKGSKVVVGAVK;
or
b)
(SEQ ID NO: 16)
KLVWGAVKGSKLVVVGAVK;
or
c)
(SEQ ID NO: 17)
KVVVGAVGKGSKVVVGAVGK;
or
d)
(SEQ ID NO: 18)
KVVVGAVGVGKGSKVWGAVGVGK;
or
e)
(SEQ ID NO: 128)
KVWGAGVKGSKVVVGAGVK;
or
f)
(SEQ ID NO: 129)
KLVWGAGVKGSKLVVVGAGVK;
or
g)
(SEQ ID NO: 130)
KVVVGAGWKGSKVWGAGWK;
or
h)
(SEQ ID NO: 131)
KVVVGAGWGKGSKVWGAGWGK;
optionally wherein the amino acid sequence comprises one or more D-amino acids and/or analogues of one or more of its amino acids, optionally wherein the N-terminal amino acid is acetylated and/or the C-terminal amino acid is amidated.

16. The molecule according to claim 1, which comprises a detectable label, a moiety that allows for isolation of the molecule, a moiety increasing the stability or half-life of the molecule, a moiety increasing the solubility of the molecule, a moiety increasing the cellular uptake of the molecule, and/or a moiety effecting targeting of the molecule to cells.

17. A method of treating a disease in a subject, wherein the disease is caused by or associated with a mutation at position 12 or 13 in human RAS protein, the method comprising administering a) the molecule according to claim 1, or b) a nucleic acid encoding said molecule.

18. (canceled)

19. The method according to claim 17, wherein the disease is a neoplastic disease, particularly cancer.

20. The method according to claim 17, wherein the disease is pancreatic ductal adenocarcinoma, colorectal adenocarcinoma, multiple myeloma, lung adenocarcinoma, skin cutaneous melanoma, uterine corpus endometrioid carcinoma, uterine carcinosarcoma, thyroid carcinoma, acute myeloid leukaemia, bladder urothelial carcinoma, gastric adenocarcinoma, cervical adenocarcinoma, head and neck squamous cell carcinoma, non-small cell lung cancer (NSCLC), or colorectal cancer.

21. A pharmaceutical composition comprising a) the molecule according to claim 1, or b) a nucleic acid encoding said molecule, wherein the molecule is a polypeptide.
